(19) United States
(12) Patent Application Publication
(10) Pub. No.: US 2003/0119053 A1
(43) Pub. Date: Jun. 26, 2003

(54) Antigen cocktails, P35, and uses thereof

(76) Inventors: Gregory T. Maine, Gurnee, IL (US); Jeffrey C. Hunt, Mundelein, IL (US); Susan Brojanac, Brookfield, WI (US); Michael Jyh-Tsing Sheu, Gurnee, IL (US); Linda E. Chovan, Kenosha, WI (US); Joan D. Tyner, Beach Park, IL (US); Lawrence V. Howard, Libertyville, IL (US); Stephen F. Parmley, San Jose, CA (US); Jack S. Remington, Menlo Park, CA (US); Fausto Araujo, Palo Alto, CA (US); Yashuhiro Suzuki, Menlo Park, CA (US); Shuli Li, San Jose, CA (US)

Correspondence Address: Steven F. Weinstock, Abbott Laboratories, D-377 / AP6D-2, 100 Abbott Park Road, Abbott Park, IL, 60064-6050, US

(21) Appl. No.: 09/728,644

(22) Filed: Dec. 1, 2000

Related U.S. Application Data
(62) Division of application No. 09/303,064, filed on Apr. 30, 1999, now Pat. No. 6,221,619.
Application No. 09/303,064 is a continuation-in-part of application No. 09/086,503, filed on May 28, 1998.

(51) Int. Cl. G01N 33/53; C07K 1/00; C07K 14/00; C07K 17/00

(52) U.S. Cl. 435/7.1; 530/350

(57) ABSTRACT

The present invention relates to combinations or mixtures of antigens which may be used in the detection of IgM and/or IgG antibodies to Toxoplasma gondii as well as to the P35 antigen which may be used to distinguish acute from chronic Toxoplasmosis. Furthermore, the present invention also relates to methods of using these combinations of antigens, antibodies raised against these combinations of antigens or against the novel P29 antigen thereof, as well as kits and vaccines containing the antigens present in the combinations.

The present application is a Continuation-In-Part of pending U.S. patent application Ser. No. 09/086,503, filed on May 28, 1998, hereby incorporated in its entirety by reference.

### BACKGROUND OF THE INVENTION

1. Technical Field

The present invention relates to combinations or mixtures of antigens which may be used in the detection of IgM or IgG antibodies to Toxoplasma gondii, as well as one antigen, in particular, which may be used to distinguish between acute and chronic infection. Furthermore, the present invention also relates to methods of using these combinations of antigens, antibodies raised against these combinations of antigens or against the novel P29 antigen thereof, as well as kits and vaccines containing the antigens present in the combinations.

2. Background Information

Toxoplasma gondii is an obligate intracellular parasite which is classified among the Coccidia. This parasite has relatively broad host range infecting both mammals and birds. The organism is ubiquitous in nature and exists in three forms: tachyzoite, cyst, and oocyst (Remington, J. S., McLeod, R., Desmonds, G., Infectious Diseases of the Fetus and Newborn Infant (J. S. Remington and J. O. Klein, Eds.), pp. 140-267, Saunders, Philadelphia (1995)). Tachyzoites, found during acute infection, are the invasive form capable of invading all nucleated mammalian cells. After the acute stage of infection, tissue cysts called bradyzoites are formed within host cells and persist within the host organism for the life of the host. Cysts are important in transmission of infection, especially in humans, as the ingestion of raw or undercooked meat can result in the ingestion of bradyzoites which can infect the individual resulting in an acute infection. Oocysts represent a stage of sexual reproduction which occurs only in the intestinal lining of the cat family from which they are excreted in the feces.

A T. gondii infection acquired through contaminated meat or cat feces in a healthy adult is often asymptomatic. In pregnant women and immunosuppressed patients, the clinical outcome can be very serious. An acute infection with T. qondii acquired during pregnancy, especially during the first trimester, can result in intrauterine transmission to the unborn fetus resulting in severe fetal and neonatal complications, including mental retardation and fetal death. Recrudesence of a previous T. gondii infection or an acute infection in an immunosuppressed individual can be pathogenic. Toxoplasmic encephalitis is a major cause of morbidity and mortality in AIDS patients. Toxoplasma infection has also been shown to be a significant cause of chorioretinitis in children and adults.

Diagnosis of infection with T. gondii may be established by the isolation of T. gondii from blood or body fluids, demonstration of the presence of the organism in the placenta or tissues of the fetus, demonstration of the presence of antigen by detection of specific nucleic acid sequences (e.g., DNA probes), or detection of T. gondii specific immunoglobulins synthesized by the host in response to infection using serologic tests.

The detection of T. gondii specific antibodies and determination of antibody titer are important tools used in the diagnosis of toxoplasmosis. The most widely used serologic tests for the diagnosis of toxoplasmosis are the Sabin-Feldman dye test (Sabin, A. B. and Feldman, H. A. (1948) Science 108, 660-663), the indirect hemagglutination (IHA) test (Jacobs, L. and Lunde, M. (1957) J. Parasitol. 43, 308-314), the IFA test (Walton, B. C. et al. (1966) Am. J. Trop. Med. Hyg. 15, 149-152), the agglutination test (Fondation Mérieux, Sérologie de I'Infection Toxoplasmique en Particulier à Son Début: Methodes et Interprétation des Résultants, Lyon, 182 pp. (1975)) and the ELISA (Naot, Y. and Remington, J. S. (1980) J. Infect. Dis. 142, 757-766). The ELISA test is one the easiest tests to perform, and many automated serologic tests for the detection of Toxoplasma specific IgM and IgG are commercially available.

The current tests for the detection of IgM and IgG antibodies in infected individuals can vary widely in their ability to detect serum antibody. Hence, there is significant inter-assay variation seen among the commercially available kits. The differences observed between the different commercial kits are caused primarily by the preparation of the antigen used for the serologic test. Most kits use either whole or sonicated tachyzoites grown in tissue culture or in mice which contain a high proportion of extra-parasitic material, for example, mammalian cells, tissue culture components, etc. Due to the lack of a purified, standardized antigen or standard method for preparing the tachyzoite antigen, it is not surprising that inter-assay variability exists resulting in different assays having different performance characteristics in terms of assay sensitivity and specificity.

Given the limitations of serologic tests employing the tachyzoite antigen, as described above, as well as the persistent problems regarding determination of onset of infection, purified recombinant antigens obtained by molecular biology are an attractive alternative in that they can be purified and standardized. In the literature, a number of Toxo genes have been cloned and expressed in a suitable host to produce immunoreactive, recombinant Toxo antigens. For example, the Toxo P22 (SAG2), P24 (GRA1), P25, P28 (GRA2), P30 (SAG1), P35 (mentioned above), P41 (GRA4), P54 (ROP2), P66 (ROP1), and the Toxo P68 antigens have been described (Prince et al. (1990) Mol. Biochem. Parasitol 43, 97-106; Cesbron-Delauw et al. (1989) Proc. Nat. Acad. Sci. 86, 7537-7541; Johnson et al. (1991) Gene 99, 127-132; Prince et al. (1989) Mol. Biochem. Parasitol. 34, 3-13; Burg et al. (1988) J. Immunol. 141, 3584-3591; Knapp et al. (1989) EPA 431541A2; Mevelec et al. (1992) Mol. Biochem. Parasitol. 56, 227-238; Saavedra et al. (1991) J. Immunol. 147, 1975-1982); EPA 751 147).

It is plausible that no single Toxo antigen can replace the tachyozoite in an initial screening immunoassay for the detection of Toxo-specific immunoglobulins. This may be due to several reasons. First, the antibodies produced during infection vary with the stage of infection, i.e., the antibodies produced by an infected individual vary over time reacting with different epitopes. Secondly, the epitopes present in a recombinant antigen may be different or less reactive than native antigen prepared from the tachyzoite depending on the host used for expression and the purification scheme employed. Thirdly, different recombinant antigens may be needed to detect the different classes of immunoglobulins produced in response to an infection, e.g., IgM, IgG, IgA and IgE.

In order to overcome the limitations of the tachyzoite antigen in terms of assay specificity and sensitivity, a search was begun for novel Toxo antigens which could be used in combination with known existing antigens in order to configure new assays for the detection of Toxo-specific immunoglobulins.

Additionally, it should be noted that the presence of IgG antibodies in a single sample of serum is sufficient to establish that the patient has been infected but does not give an indication as to when the infection occurred. In the United States, there is no systematic serological screening program in pregnant women, whereas in countries such as France and Austria, sera are obtained at regular intervals throughout gestation in women who are seronegative when first tested. In the United States, a decision regarding whether the woman was recently infected thereby placing her fetus at risk, is often made from results in a single sample of serum. However, it is critical in pregnant women to determine as accurately as possible if they acquired their infection just prior to or during gestation. For this reason, the presence of IgG antibodies in a pregnant woman often leads to additional serological testing to attempt to determine if the infection was acquired during pregnancy or in the distant past (Remington et al., 1995, Toxoplasmosis, 4^{th} ed., Coord. Ed., Remington, J. S., W. B. Saunders, Philadelphia, Pa.). Of the recommended additional serological tests, those that demonstrate the presence of IgM antibodies are most frequently used. However, since IgM antibodies may remain detectable for more than one year after initial infection, demonstration of these antibodies cannot be used to prove recently acquired infection (Liesebfeld et al., Journal of Clinical Microbiology 35:174-78 (1997); Wilson et al., Journal of Clinical Microbiology 35:3112-15 (1997); Wong et al., Clinical Infectious Diseases 18:853-62 (1994)). Because accurate diagnosis of the recently acquired infection in pregnant women is important for clinical management of both the mother and her fetus, a search has continued for better diagnostic methods (Remington et al., 1995, Toxoplasmosis, 4^{th} ed., Coord. Ed., J. S. Remington, W. B. Saunders, Philadelphia, Pa.; Wong et al., supra).

In previous studies, it was observed that a 35 kDa antigen was detected in immunoblots of tachyzoite extracts probed with serum from individuals early after they became infected with T. gondii and postulated that this antigen might prove useful for detection of the acute stage of the infection (Potasman et al., Journal of Infectious Diseases 154:650-57 (1986); Potasman et al., Journal of Clinical Microbiology 25:1926-31 (1987)). Thus, a gene in the GenBank sequence database for T. gondii putatively identified as “P35” was selected for cloning, expression, and evaluation of a corresponding recombinant protein for its capacity to detect serum antibodies during the early phase of the infection. This antigen will be described in further detail below.

Additionally, it was determined that a portion of one of these antigens (i.e., P35) could be utilized to distinguish between acute and chronic infection.

### SUMMARY OF THE INVENTION

The present invention includes a composition comprising Toxoplasma gondii antigens P29, P30 and P35 as well as a composition comprising Toxoplasma gondii antigens P29, P35 and 66. These compositions may be used as diagnositic reagents, and the antigens within these compositions may be produced either recombinantly or synthetically.

Additionally, the present invention includes an isolated nucleic acid sequence represented by SEQ ID NO: 26 and a purified polypeptide having the amino acid sequence represented by SEQ ID NO: 27. The present invention also includes a polyclonal or monoclonal antibody directed against the purified polypeptide.

The present invention also encompasses a method for detecting the presence of IgM antibodies to Toxoplasma gondii in a test sample. This method comprises the steps of: a) contacting the test sample suspected of containing the IgM antibodies with a composition comprising P29, P35 and P66; and b) detecting the presence of the IgM antibodies.

Furthermore, the present invention includes an additional method for detecting the presence of IgM antibodies to Toxoplasma gondii in a test sample. This method comprises the steps of: a) contacting the test sample suspected of containing the IgM antibodies with a composition comprising antigen P29, P35 and P66 for a time and under conditions sufficient for the formation of IgM antibody/antigen complexes; b) adding a conjugate to the resulting IgM antibody/antigen complexes for a time and under conditions sufficient to allow the conjugate to bind to the bound antibody, wherein the conjugate comprises an antibody attached to a signal generating compound capable of generating a detectable signal; and c) detecting the presence of IgM antibodies which may be present in the test sample by detecting a signal generated by the signal generating compound.

Moreover, the present invention also includes a method for detecting the presence of IgG antibodies to Toxoplasma gondii in a test sample. This method comprises the steps of: a) contacting the test sample suspected of containing the IgG antibodies with a composition comprising P29, P30 and P35; and b) detecting the presence of the IgG antibodies.

Additionally, the present invention encompasses another method for detecting the presence of IgG antibodies to Toxoplasma gondii in a test sample. This method comprising the steps of: a) contacting said test sample suspected of containing the IgG antibodies with a composition comprising antigen P29, P30 and P35 for a time and under conditions sufficient for formation of IgG antibody/antigen complexes; b) adding a conjugate to resulting IgG antibody/antigen complexes for a time and under conditions sufficient to allow the conjugate to bind to bound antibody, wherein the conjugate comprises an antibody attached to a signal generating compound capable of generating a detectable signal; and c) detecting IgG antibodies which may be present in said test sample by detecting a signal generated by said signal generating compound.

Additionally, the present invention includes another method for detecting the presence of IgM antibodies to Toxoplasma gondii in a test sample. This method comprises the steps of: a) contacting the test sample suspected of containing the IgM antibodies with anti-antibody specific for the IgM antibodies for a time and under conditions sufficient to allow for formation of anti-antibody/IgM antibody complexes; b) adding a conjugate to resulting anti-antibody/IgM antibody complexes for a time and under conditions sufficient to allow the conjugate to bind to bound antibody, wherein the conjugate comprises P29, P35 and P66, each attached to a signal generating compound capable of generating a detectable signal; and c) detecting IgM antibodies which may be present in the test sample by detecting a signal generated by the signal generating compound.

Another method for detecting the presence of IgG antibodies to Toxoplasma gondii in a test sample, encompassed by the present invention, comprises the steps of: a) contacting the test sample suspected of containing the IgG antibodies with anti-antibody specific for the IgG antibodies for a time and under conditions sufficient to allow for formation of anti-antibody/IgG antibody complexes; b) adding a conjugate to resulting anti-antibody/IgG antibody complexes for a time and under conditions sufficient to allow the conjugate to bind to bound antibody, wherein the conjugate comprises P29, P30 and P35, each attached to a signal generating compound capable of generating a detectable signal; and c) detecting IgG antibodies which may be present in the test sample by detecting a signal generated by the signal generating compound.

Also, the present invention includes a vaccine comprising: 1) Toxoplasma gondii antigens P29, P30 and P35 and 2) a pharmaceutically acceptable adjuvant as well as a vaccine comprising: 1) Toxoplasma gondii antigens P29, P35 and P66 and 2) a pharmaceutically acceptable adjuvant.

Additionally, the present invention includes a kit for determining the presence of IgM antibodies to Toxoplasma gondii in a test sample comprising: a) a composition comprising Toxoplasma gondii antigens P29, P35 and P66 and b) a conjugate comprising an antibody attached to a signal generating compound capable of generating a detectable signal.

The present invention also includes a kit for determining the presence of IgG antibodies to Toxoplasma gondii in a test sample comprising: a) a composition comprising Toxoplasma gondii antigens P29, P30 and P35 and b) a conjugate comprising an antibody attached to a signal generating compound capable of generating a detectable signal.

An additional kit for determining the presence of IgM antibodies to Toxoplasma gondii in a test sample, encompassed by the present invention, comprises: a) an anti-antibody specific for IgM antibody and b) a composition comprising Toxoplasma gondii antigens P29, P35 and P66.

The present invention also includes a kit for determining the presence of IgM antibodies to Toxoplasma gondii in a test sample comprising: a) an anti-antibody specific for IgM antibody and b) a conjugate comprising: 1) Toxoplasma gondii antigens P29, P35 and P66, each attached to 2) a signal generating compound capable of generating a detectable signal.

Additionally, the present invention includes a kit for determining the presence of IgG antibodies to Toxoplasma gondii in a test sample comprising: a) an anti-antibody specific for IgG antibody and b) a composition comprising Toxoplasma gondii antigens P29, P30 and P35.

The present invention also includes an additional kit for determining the presence of antibodies to Toxoplasma gondii in a test sample comprising: a) an anti-antibody specific for IgG antibody and b) a conjugate comprising: 1) Toxoplasma gondii antigens P29, P30 and P35, each attached to 2) a signal generating compound capable of generating a detectable signal.

Additionally, the present invention includes a method for detecting the presence of IgM antibodies to Toxoplasma gondii in a test sample comprising the steps of: (a) contacting the test sample suspected of containing IgM antibodies with anti-antibody specific for the IgM antibodies for a time and under conditions sufficient to allow for formation of anti-antibody IgM complexes; (b) adding antigen to resulting anti-antibody/IgM complexes for a time and under conditions sufficient to allow the antigen to bind to bound IgM antibody, the antigen comprising a mixture of P29, P35 and P66; and (c) adding a conjugate to resulting anti-antibody/IgM/antigen complexes, the conjugate comprising a composition comprising monoclonal or polyclonal antibody attached to a signal generating compound capable of generating a detectable signal; and (d) detecting IgM antibodies which may be present in the test sample by detecting a signal generated by the signal generating compound.

The present invention also includes a method for detecting the presence of IgG antibodies to Toxoplasma gondii in a test sample comprising the steps of: (a) contacting the test sample suspected of containing IgG antibodies with anti-antibody specific for said IgG antibodies for a time and under conditions sufficient to allow for formation of anti-antibody IgG complexes; (b) adding antigen to resulting anti-antibody/IgG complexes for a time and under conditions sufficient to allow said antigen to bind to bound IgG antibody, the antigen comprising a mixture of P29, P30 and P35; and (c) adding a conjugate to resulting anti-antibody/IgG/antigen complexes, the conjugate comprising a composition comprising monoclonal or polyclonal antibody attached to a signal generating compound capable of generating a detectable signal; and (d) detecting IgG antibodies which may be present in the test sample by detecting a signal generated by the signal generating compound.

A further method for detecting the presence of IgM and IgG antibodies to Toxoplasma gondii in a test sample, included within the present invention, comprises the steps of: a) contacting the test sample suspected of containing the IgM and IgG antibodies with a composition comprising antigen P29, P30, P35 and P66 for a time and under conditions sufficient for the formation of IgM antibody/antigen complexes and IgG antibody/antigen complexes; b) adding a conjugate to the resulting IgM antibody/antigen complexes and IgG antibody/antigen complexes for a time and under conditions sufficient to allow the conjugate to bind to the bound IgM and IgG antibody, wherein said conjugate comprises an antibody attached to a signal generating compound capable of generating a detectable signal; and c) detecting the presence of IgM and IgG antibodies which may be present in the test sample by detecting a signal generated by the signal generating compound.

The present invention also includes a method for detecting the presence of IgM and IgG antibodies to Toxoplasma gondii in a test sample comprising the steps of: a) contacting the test sample suspected of containing the IgM and IgG antibodies with anti-antibody specific for said IgM antibodies and the IgG antibodies for a time and under conditions sufficient to allow for formation of anti-antibody/IgM antibody complexes and anti-antibody/IgG antibody complexes; b) adding a conjugate to resulting anti-antibody/IgM antibody complexes and resulting anti-antibody/IgG antibody complexes for a time and under conditions sufficient to allow the conjugate to bind to bound antibody, wherein the conjugate comprises P29, P30, P35 and P66, each attached to a signal generating compound capable of generating a detectable signal; and c) detecting IgM and IgG antibodies which may be present in the test sample by detecting a signal generated by the signal generating compound.

The present invention also includes a method for detecting the presence of IgM and IgG antibodies to Toxoplasma gondii in a test sample comprising the steps of: (a) contacting the test sample suspected of containing IgM and IgG antibodies with anti-antibody specific for the IgM antibodies and with anti-antibody specific for the IgG antibodies for a time and under conditions sufficient to allow for formation of anti-antibody/IgM complexes and anti-antibody/IgG complexes; (b) adding antigen to resulting anti-antibody/IgM complexes and resulting anti-antibody/IgG complexes for a time and under conditions sufficient to allow the antigen to bind to bound IgM antibody and bound IgG antibody, the antigen comprising a mixture of P29, P30, P35 and P66; and (c) adding a conjugate to resulting anti-antibody/IgM/antigen complexes and anti-antibody/IgG/antigen complexes, the conjugate comprising a composition comprising monoclonal or polyclonal antibody attached to a signal generating compound capable of generating a detectable signal; and (d) detecting IgM and IgG antibodies which may be present in the test sample by detecting a signal generated by the signal generating compound.

Additionally, the present invention encompasses a method of producing monoclonal antibodies comprising the steps of:

a) injecting a non-human mammal with an antigen;

b) administering a composition comprising antibiotics to the non-human mammal;

c) fusing spleen cells of the non-human mammal with myeloma cells in order to generate hybridomas; and

d) culturing the hybridomas under sufficient time and conditions such that the hybridomas produce monoclonal antibodies.

The antigen utilized may be derived from, for example, T. gondii.

The present invention also encompasses a composition comprising the isolated nucleic acid sequence illustrated in FIG. 11 or a fragment thereof.

Additionally, the present invention includes a composition comprising amino acids 1-135 of P35. Either of the two compositions may be a diagnostic reagent. The present invention also includes portions or fragments of P35 which have the same antigenic properties as the region of P35 which consists of amino acids 1-135.

The present invention also includes a method for distinguishing between acute and chronic Toxoplasmosis in a patient suspected of having either acute or chronic Toxoplasmosis. This method comprises the steps of: a) contacting a test sample, from the patient, with a composition comprising amino acids 1-135 of P35; and b) detecting the presence of IgG antibodies, presence of the IgG antibodies indicating acute Toxoplasmosis in the patient and lack of the IgG antibodies indicating chronic Toxoplasmosis in the patient.

Further, the present invention includes a kit for distinguishing between acute and chronic Toxoplasmosis in a patient suspected of having either acute Toxoplasmosis or chronic Toxoplasmosis comprising: a) a composition comprising amino acids 1-135 of Toxoplasma gondii antigen P35; and b) a conjugate comprising an antibody attached to a signal generating compound capable of generating a detectable signal.

Additionally, the present invention encompasses a kit for distinguishing between acute and chronic Toxoplasmosis in a patient suspected of having either acute Toxoplasmosis or chronic Toxoplasmosis comprising:

a) an anti-antibody specific for IgG antibody; and b) a conjugate comprising amino acids 1-135 of Toxoplasma gondii antigen P35 attached to a signal generating compound capable of generating a detectable signal.

All U.S. patents and publications referred to herein are hereby incorporated in their entirety by reference.

### BRIEF DESCRIPTION OF THE DRAWINGS

FIG. 1 represents the DNA sequence [SEQ ID NO: 23] of nucleotides 1-1268 and the corresponding amino acid sequence [SEQ ID NO: 24] of plasmid pGM613.

FIG. 2 represents the DNA sequence [SEQ ID NO: 25] of nucleotides 1-477 of plasmid pTXG1-2.

FIG. 3 represents the composite DNA sequence [SEQ ID NO: 26] of nucleotides 1-1648 and the corresponding amino acid sequence [SEQ ID NO: 27] for the P29 gene.

FIG. 4 is a schematic representation of (A) the construction of plasmid pEE2; (B) the nucleotide sequence [SEQ ID NO: 28] and the corresponding amino acid sequence [SEQ ID NO: 49] of the polylinker to be removed from pEE1 by digestion with BglII; and (C) the nucleotide sequence [SEQ ID NO: 29] and the corresponding amino acid sequence [SEQ ID NO: 50] of the synthetic DNA to be introduced into the BglII site of pEE1 to generate plasmid pEE2.

FIG. 5 is a schematic representation of (A) the construction of plasmid pEE3; and (B) the nucleotide sequence [SEQ ID NO: 32] and the corresponding amino acid sequence [SEQ ID NO: 51] of the synthetic DNA polylinker to be introduced into the StuI/MluI sites of pEE2 to generate plasmid pEE3.

FIG. 6 is a schematic representation of the construction of plasmid pToxo-P29.

FIG. 7 illustrates the DNA sequence [SEQ ID NO: 37] of nucleotides 1-4775 and the corresponding amino acid sequence [SEQ ID NO: 52] of the CKS-P29-CKS fusion protein of plasmid pToxo-P29.

FIG. 8 is a schematic representation of the construction of plasmid pToxo-P30.

FIG. 9 represents the DNA sequence [SEQ ID NO: 40] of nucleotides 1-4910 and the corresponding amino acid sequence [SEQ ID NO: 53] of the CKS-P30-CKS fusion protein of plasmid pToxo-P30.

FIG. 10 is a schematic representation of the construction of plasmid pToxo-P35S.

FIG. 11 illustrates the DNA sequence [SEQ ID NO: 45] of nucleotides 1-4451 and the corresponding amino acid sequence [SEQ ID NO: 54] of the CKS-P35-CKS fusion protein of plasmid pToxo-P35S. The first 171 amino acids represent a portion of CKS, the next 135 amino acids represent amino acids 1-135 of P35, and the remaining amino acids represent the remainder of CKS.

FIG. 12 is a schematic representation of the construction of plasmid pToxo-P66g.

FIG. 13 represents the DNA sequence [SEQ ID NO: 48] of nucleotides 1-5258 and the corresponding amino acid sequence [SEQ ID NO: 55] of the CKS-P66-CKS fusion protein of plasmid pToxo-P66g.

FIG. 14 illustrates the reactivity of T. gondii antibodies with rpToxo-P35S and with CKS preparations. Strips of the rpToxo-P35S blot (A) or CKS blot (B) were stained with amido black (lane 1), monoclonal antibody against CKS protein (lane 2), pooled Group I sera (lane 3), pooled Group II sera (lane 4) or pooled Group III sera (lane 5). The position of rpToxo-P35S (approximately 54 kD) and the CKS protein (approximately 34 kD) are indicated with arrows. Molecular weight markers are indicated on the side. Cross-reactive bands in the CKS preparation are also indicated by arrows.

FIG. 15 illustrates ELISA readings in 41 Group I sera. Dark columns are OD 450 readings with rpToxo-P35S preparation and light columns are readings with the CKS preparation.

FIG. 16 represents ELISA readings in 50 Group II sera. Dark columns are readings with rpToxo-P35S preparation, and light columns are readings with the CKS preparation.

FIG. 17 illustrates rpToxo-P35S ELISA readings of 41 Group I sera after subtraction of the readings of the same sera in the control ELISA. The horizonal lines represent the cut-off values of 0.014 (mean +2 SD) and 0.019 (mean +3 SD) obtained as described in the Examples.

FIG. 18 represents rpToxo-P35S ELISA readings of 50 Group I sera after subtraction of the readings of the same seria in the control ELISA. The horizontal lines represent the cut-off values as in FIG. 17.

### DETAILED DESCRIPTION OF THE INVENTION

The difficulties of known assays for the detection of IgG and IgM antibodies to T. gondii have been described, in detail, above. Thus, there was a need to discover immunoassays which could accurately detect the presence of such antibodies in positive serum, thereby eliminating the problem of false negative or false positive tests. The present invention provides such needed immunoassays and, in particular, combinations of antigens which accurately detect the presence of IgG or IgM antibodies in human sera.

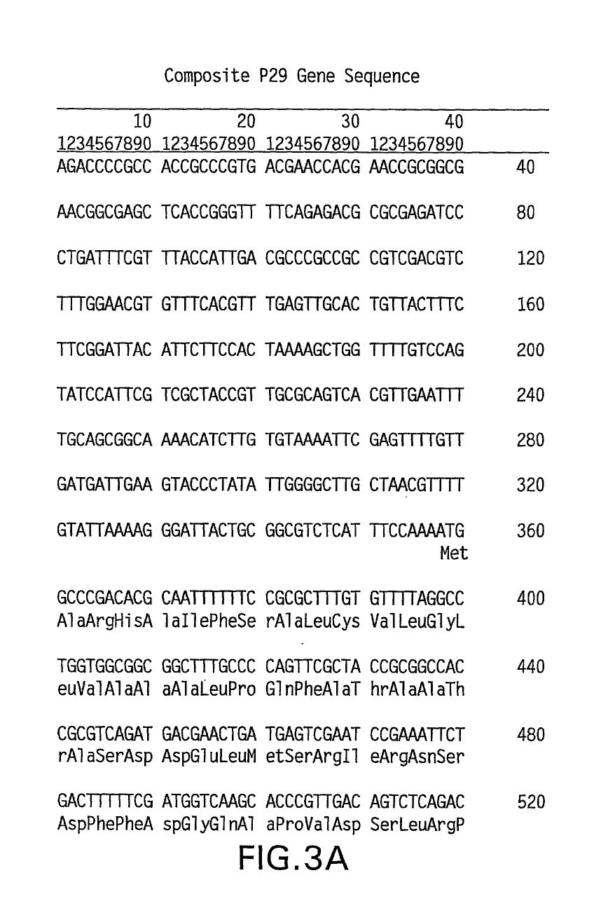

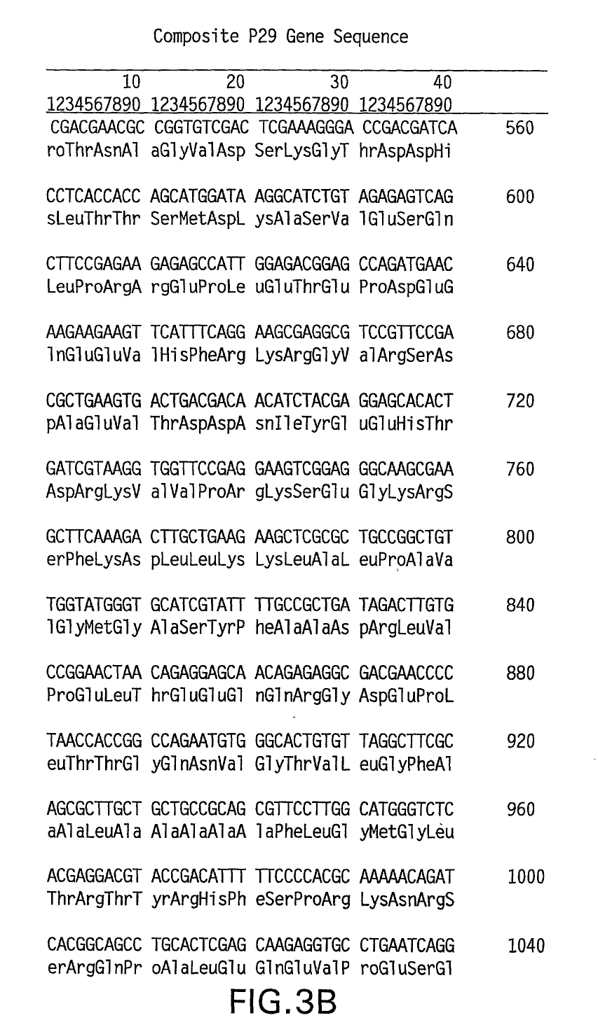

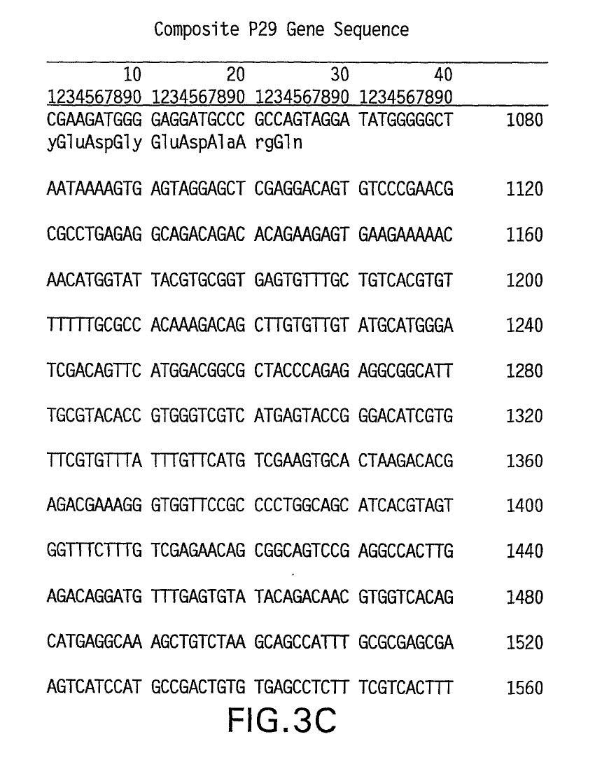

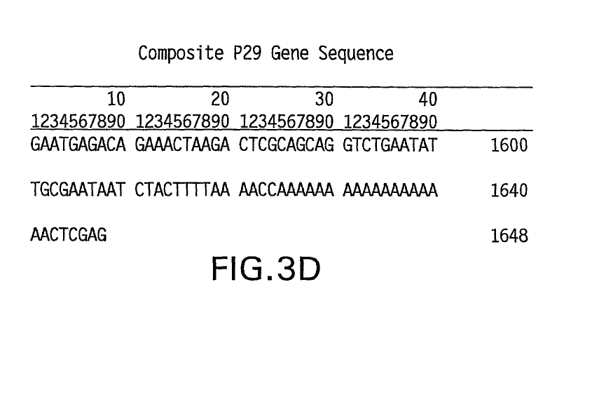

In particular, the present invention includes a novel antigen which, for purposes of the present invention, is referred to as P29. The nucleotide sequence of the gene encoding this antigen is shown in FIG. 3 and is represented by SEQ ID NO. 26. The amino acid sequence of this antigen is shown in FIG. 3 and is represented by SEQ ID NO-. 27.

P29, a dense granule proten, when used in combination with other known antigens, may accurately detect the presence of IgG or IgM in human sera. In particular, P29, when used in combination with other known antigens, may replace the tachyzoite previously used in assays for T. gondii antibodies.

Furthermore, the present invention also includes a polyclonal or monoclonal antibody raised against P29. Such an antibody may be used, for example, in an immunoassay, a vaccine, a kit, or for research purposes.

The present invention also encompasses a composition or mixture comprising the following three antigens: P29, P30 and P35. This combination or mixture of antigens may be utilized for the detection of IgG in IgG-positive sera (i.e., as a diagnostic reagent). Furthermore, the antigens may be produced either recombinantly or synthetically. Additionally, the present invention also includes a composition comprising antibodies raised against these antigens.

The present invention also includes a composition or mixture comprising the following three antigens: P29, P35 and P66. This combination or mixture of antigens may be used for the detection of IgM in IgM-positive sera (i.e., as a diagnostic reagent), and the antigens may be produced either recombinantly or synthetically. Furthermore, the present invention also includes a composition comprising antibodies raised against these antigens.

If, in fact, one wishes to measure both the titer of IgM and IgG in an individual, then a composition or mixture of antigens P29, P30, P35 and P66 may be utilized in an immunoassay. Such a combination of antigens is also included within the scope of the present invention.

The present invention also includes methods of detecting IgM and/or IgG using the combinations of antigens described above. More specifically, there are two basic types of assays, competitive and non-competitive (e.g., immunometric and sandwich). In both assays, antibody or antigen reagents are covalently or non-covalently attached to the solid phase. Linking agents for covalent attachment are known and may be part of the solid phase or derivatized to it prior to coating. Examples of solid phases used in immunoassays are porous and non-porous materials, latex particles, magnetic particles, microparticles, beads, membranes, microtiter wells and plastic tubes. The choice of solid phase material and method of labeling the antigen or antibody reagent are determined based upon desired assay format performance characteristics. For some immunoassays, no label is required. For example, if the antigen is on a detectable particle such as a red blood cell, reactivity can be established based upon agglutination. Alternatively, an antigen-antibody reaction may result in a visible change (e.g., radial immunodiffusion). In most cases, one of the antibody or antigen reagents used in an immunoassay is attached to a signal generating compound or “label”. This signal generating compound or “label” is in itself detectable or may be reacted with one or more additional compounds to generate a detectable product. Examples of such signal generating compounds include chromogens, radioisotopes (e.g., 125I, 131I, 32P, 3H, 35S, and 14C), fluorescent compounds (e.g., fluorescein, rhodamine), chemiluminescent compounds, particles (visible or fluorescent), nucleic acids, complexing agents, or catalysts such as enzymes (e.g., alkaline phosphatase, acid phosphatase, horseradish peroxidase, beta-galactosidase, and ribonuclease). In the case of enzyme use, addition of chromo-, fluoro-, or lumo-genic substrate results in generation of a detectable signal. Other detection systems such as time-resolved fluorescence, internal-reflection fluorescence, amplification (e.g., polymerase chain reaction) and Raman spectroscopy are also useful.

There are two general formats commonly used to monitor specific antibody titer and type in humans: (1) antigen is presented on a solid phase, as described above, the human biological fluid containing the specific antibodies is allowed to react with the antigen, and then antibody bound to antigen is detected with an anti-human antibody coupled to a signal generating compound and (2) an anti-human antibody is bound to the solid phase, the human biological fluid containing specific antibodies is allowed to react with the bound antibody, and then antigen attached to a signal generating compound is added to detect specific antibody present in the fluid sample. In both formats, the anti-human antibody reagent may recognize all antibody classes, or alternatively, be specific for a particular class or subclass of antibody, depending upon the intended purpose of the assay. These assays formats as well as other known formats are intended to be within the scope of the present invention and are well known to those of ordinary skill in the art.

In particular, two illustrative examples of an immunometric antibody-capture based immunoassay are the Imx Toxo IgM and Toxo IgG antibody assays manufactured by Abbott Laboratories (Abbott Park, Ill.). Both assays are automated Microparticle Enzyme Immunoasssays (MEIA) which measure antibodies to Toxoplasma gondii (T. gondii) in human serum or plasma (Safford et al., J. Clin. Pathol. 44:238-242 (1991)). One assay quantitatively measures IgM antibodies, indicative of recent exposure or acute infection, and the other assay quantitatively measures IgG, indicative of chronic or past infection. These assays use microparticles coated with T. gondii antigens as the solid phase. In particular, specimen is added to the coated microparticles to allow antibodies specific for T. gondii to bind. Subsequently, an alkaline phosphatase conjugated anti-human IgM (or anti-human IgG) is added that specifically binds to IgM (or IgG) class antibodies complexed to the T. gondii antigens. Following addition of a suitable substrate (e.g., 4-methyumbelliferyl phosphate), the rate of enzyme-catalyzed turnover is monitored based upon fluorescence.

The mixture of P29, P30 and P35 may be used in the IgG Abbott immunoassay, and the mixture of P29, P35 and P66 may be utilized in the IgM Abbott immunoassay. Additionally, A mixture of P29, P30, P35, and P66 may be utilized in either assay, if desired. Furthermore, it must be noted that other non-Abbott assays or platforms may also be utilized, with each of the combinations of antigens (i.e., 3 or 4 antigens), for purposes of the present invention.

Thus, the present invention includes a method of detecting IgM antibodies in a test sample comprising the steps of: (a) contacting the test sample suspected of containing the IgM antibodies with P29, P35 and P66; (b) detecting the presence of IgM antibodies present in the test sample. More specifically, the present invention includes a method of detecting IgM antibodies in a test sample comprising the steps of: (a) contacting the test sample suspected of containing the IgM antibodies with P29, P35 and P66 for a time and under conditions sufficient to allow the formation of IgM antibody/antigen complexes; (b) adding a conjugate to the resulting IgM antibody/antigen complexes for a time and under conditions sufficient to allow the conjugate to bind to the bound antibody, the conjugate comprising an antibody (directed against the IgM) attached to a signal generating compound capable of generating a detectable signal; (c) detecting the presence of the IgM antibody which may be present in the test sample by detecting the signal generated by the signal generating compound. A control or calibrator may also be used which binds to the antigens. Furthermore, the method may also comprise the use of P30 in addition P29, P35 and P66.

In each of the above assays, IgG may be detected by substituting the P29, P35 and P66 mixture with a P29, P30 and P35 mixture. Additionally, the antibody in the conjugate will be directed against IgG rather than IgM. Additionally, if one wishes to detect both IgM and IgG antibodies, P29, P30, P35 and P66 may be utilized in the immunoassay. Furthermore, if desired, one may also add P66 to the assay, even if detection of antibodies to only IgG is required.

Additionally, the present invention also includes a method for detecting the presence of IgM which may be present in a test sample. This method comprises the steps of: (a) contacting the test sample suspected of containing IgM antibodies with anti-antibody specific for the IgM, for a time and under conditions sufficient to allow for formation of anti-antibody/IgM complexes and (b) detecting the presence of IgM which may be present in the test sample. (Such anti-antibodies are commercially available and may be created, for example, by immunizing a mammal with purified mu-chain of the antibody.) More specifically, this method may comprise the steps of: (a) contacting the test sample suspected of containing the IgM antibodies with anti-antibody specific for the IgM, under time and conditions sufficient to allow the formation of anti-antibody/IgM complexes; (b) adding a conjugate to the resulting anti-antibody/IgM complexes for a time and under conditions sufficient to allow the conjugate to bind to the bound antibody, the conjugate comprising P29, P35 and P66, each being attached to a signal generating compound capable of generating a detectable signal; and (c) detecting the presence of the IgM antibodies which may be present in the test sample by detecting the signal generated by the signal generating compound. A control or calibrator may be used which comprises antibody to the anti-antibody. Furthermore, the conjugate may also comprise P30, if desired.

In each of the above assays, IgG may be detected by substituting the P29, P35 and P66 mixture with a P29, P30 and P35 mixture. Also, anti-antibody specific for IgG will be used. Additionally, if one wishes to detect both IgM and IgG antibodies, P29, P30, P35 and P66 may be utilized in the immunoassay. Moreover, even if one wishes to detect IgG only, P66 may also be added to the assay, if desired.

The present invention also encompasses a third method for detecting the presence of IgM in a test sample. This method comprises the steps of: (a) contacting the test sample suspected of containing IgM antibodies with anti-antibody specific for the IgM, under time and conditions sufficient to allow the formation of anti-antibody IgM compelxes; (b) adding antigen to the resulting anti-antibody/IgM complexes for a time and under conditions sufficient to allow the antigen to bind to the bound IgM antibody, the antigen comprising a mixture of P29, P35 and P66; and (c) adding a conjugate to the resulting anti-antibody/IgM/antigen complexes, the conjugate comprising a composition comprising monoclonal or polyclonal antibody attached to a signal generating compound capable of detecting a detectable signal, the monoclonal or polyclonal antibody being directed against the antigen; and (d) detecting the presence of the IgM antibodies which may be present in the test sample by detecting the signal generated by the signal generating compound. Again, a control or calibrator may be used which comprises antibody to the anti-antibody. The antigen mixture may further comprise P30, if desired.

In this method, IgG may be detected by substituting the P29, P35 and P66 mixture with a P29, P30 and P35 mixture and utilizing anti-antibody specific for IgG. However, if one wishes to detect both IgM and IgG antibodies, P29, P30, P35 and P66 may be utilized in the immunoassay. Even if one wishes to detect IgG alone, the assay may further comprise the use of P66.

It should also be noted that all of the above methods may be used to detect IgA antibodies (with an alpha-specific conjugate) and/or IgE antibodies (with an epsilon-specific conjugate) should such detection be desired.

Additionally, the present invention also includes a vaccine comprising a mixture of P29, P30 and P35 antigens and a pharmaceutically acceptable adjuvant. Such a vaccine may be administered if one desires to raise IgG antibodies in a mammal. The present invention also includes a vaccine comprising a mixture of P29, P35 and P66 antigens and a pharmaceutically acceptable adjuvant (e.g., Freund's adjuvant or Phosphate Buffered Saline). Such a vaccine may be administered if one desires to raise IgM antibodies in a mammal. Additionally, the present invention also includes a vaccine comprising a mixture of P29, P30, P35 and P66 antigens as well as a pharmaceutically acceptable adjuvant. This vaccine should be administered if one desires to raise both IgM and IgG antibodies in a mammal.

Kits are also included within the scope of the present invention. More specifically, the present invention includes kits for determining the presence of IgG and/or IgM. In particular, a kit for determining the presence of IgM in a test sample comprises a) a mixture of P29, P35 and P66; and b) a conjugate comprising an antibody (directed against IgM) attached to a signal generating compound capable of generating a detectable signal. The kit may also contain a control or calibrator which comprises a reagent which binds to P29, P35 and P66.

Again, if one desires to detect IgG, rather than IgM, the kit will comprise a mixture of P29, P30 and P35, rather than P29, P35 and P66, as well as an antibody directed against IgG. If one wishes to detect both IgM and IgG, the kit will comprise P29, P30, P35 and P66.

The present invention also includes another type of kit for detecting IgM and/or IgG in a test sample. If utilized for detecting the presence of IgM, the kit may comprise a) an anti-antibody specific for IgM, and b) a mixture of antigens P29, P35 and P66. A control or calibrator comprising a reagent which binds to P29, P35 and P66 may also be included. More specifically, the kit may comprise a) an anti-antibody specific for IgM, and b) a conjugate comprising P29, P35 and P66, the conjugate being attached to a signal generating compound capable of generating a detectable signal. Again, the kit may also comprise a control of calibrator comprising a reagent which binds to P29, P35 and P66.

Additionally, if one desires to detect IgG, rather than IgM, the kit will comprise a mixture of P29, P30 and P35, rather than P29, P35 and P66, as well as anti-antibody specific for IgG. If one wishes to detect both IgM and IgG, the kit may comprise P29, P30, P35 and P66.

Furthermore, the present invention also encompasses a method of distinguishing between acute and chronic infection by use of a portion of the P35 antigen. An individual may be said to have “an acute infection” if the individual has seroconverted to Toxo IgG recently, perhaps within approximately the last 9 months. An acute infection is characterized by at least one of the following: high IgG titer in the Sabin Feldman Dye Test, positive IgM in a double-sandwich IgM ELISA, positive IgA in a double-sandwich IgM ELISA and acute patterns in a Differential Agglutination Test (HS/AC). In contrast, an individual may be said to have “a chronic infection” if the individual has not seroconverted to Toxo IgG recently. A chronic infection is characterized by at least one of the following: low IgG titer in the Sabin Feldman Dye Test, presence or absence of Toxo IgM antibodies (depending upon the commercial test utilized) and chronic patterns in a Differential Agglutination Test (HS/AC).

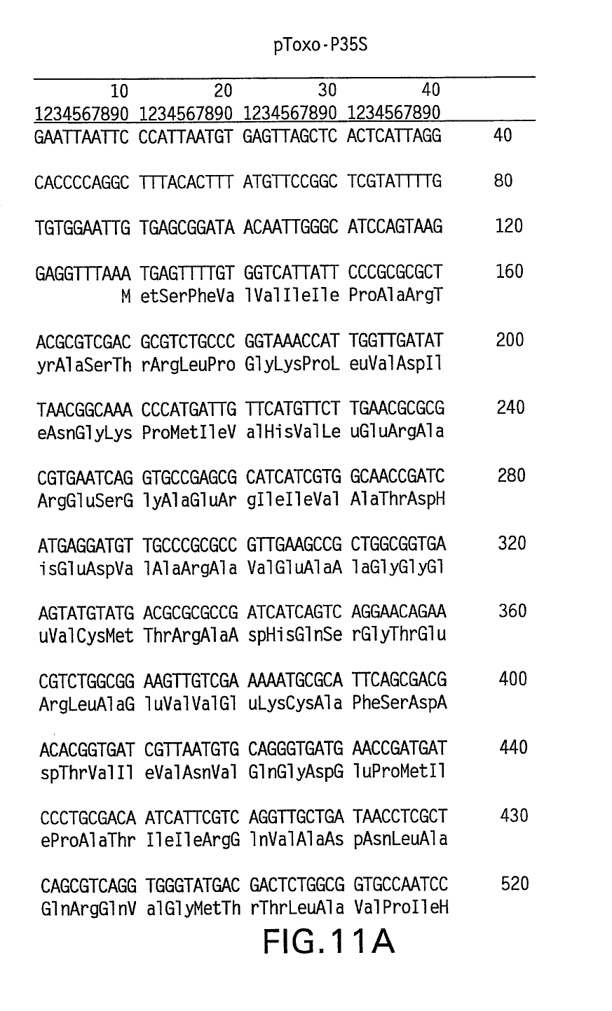

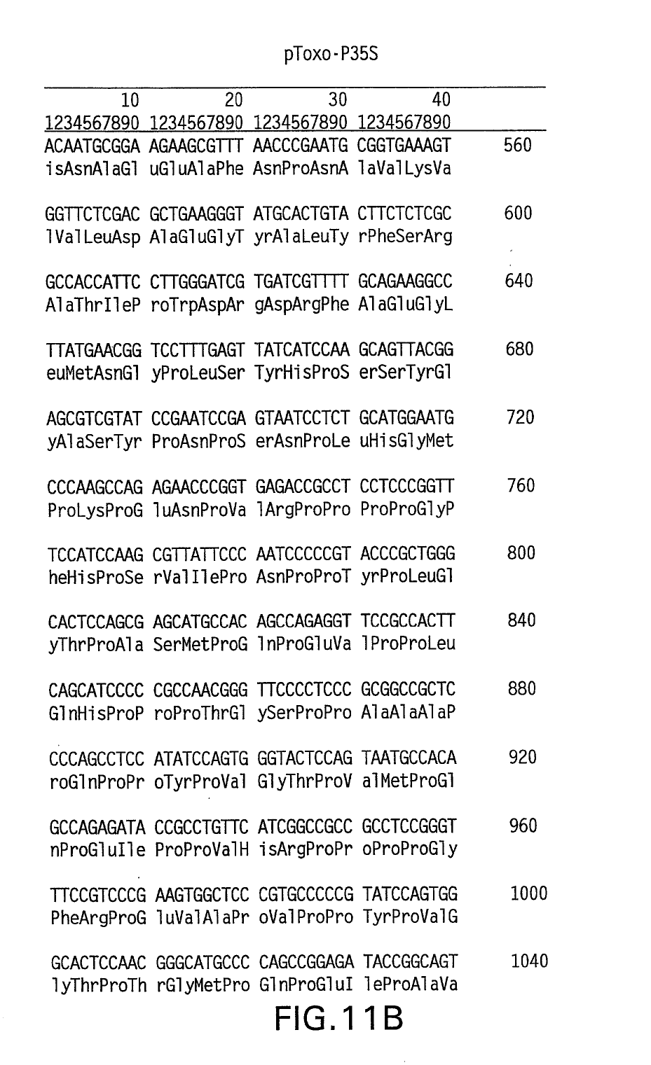

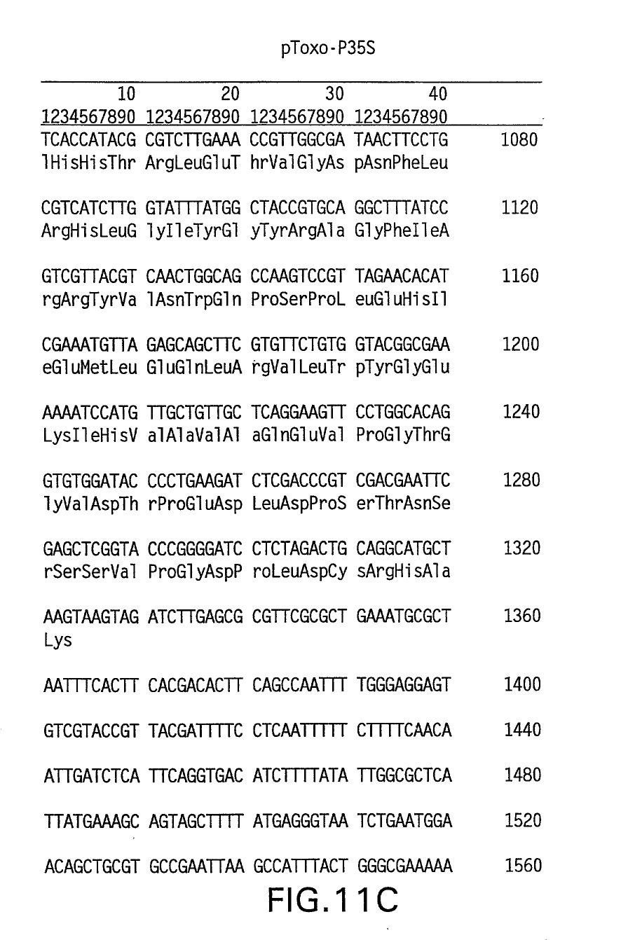

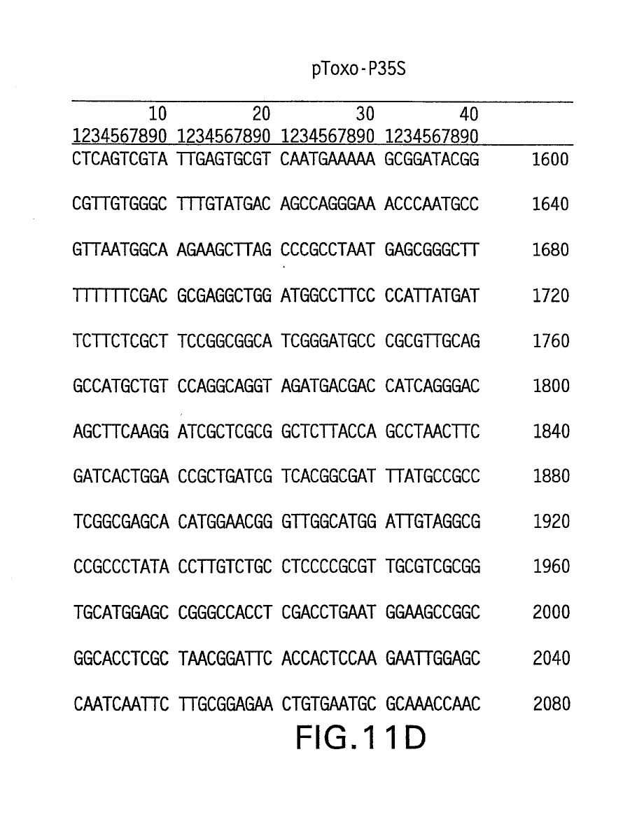

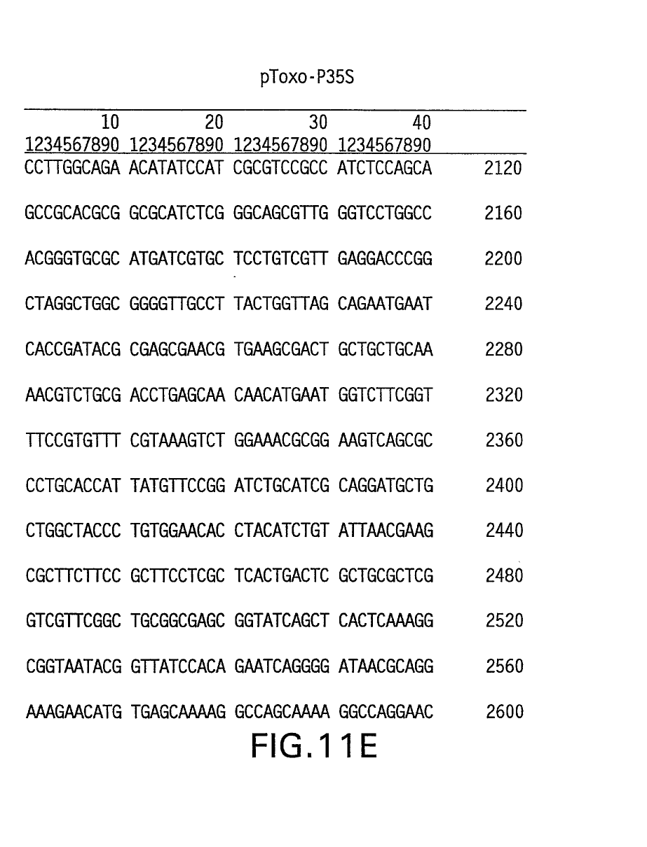

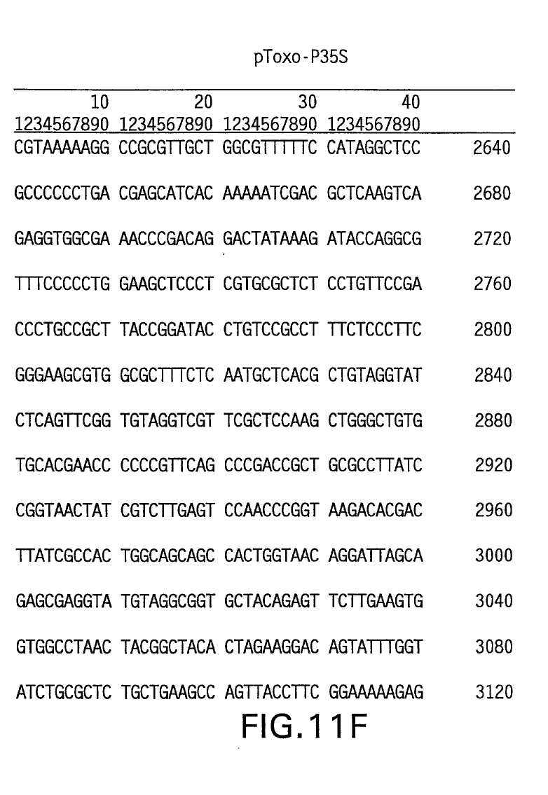

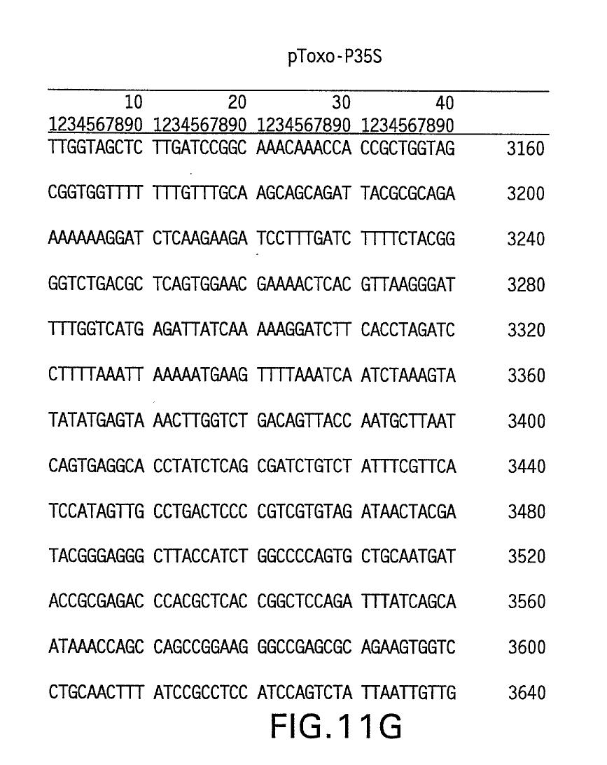

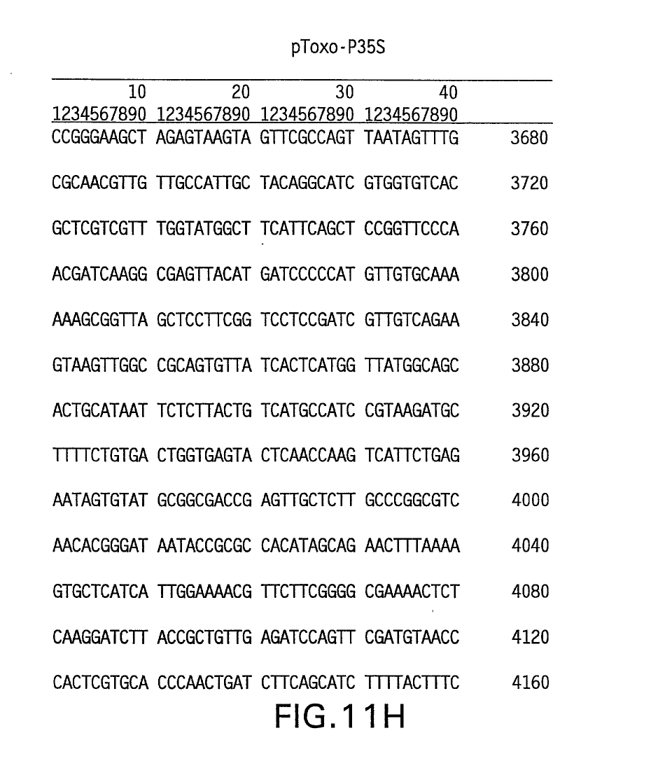

The difficulties and limitations of conventional serological assays, which detect IgM or IgG antibodies to T. gondii using the tachyzoite antigen, in distinguishing an acute toxoplasmosis from a chronic toxoplasmosis, have been described, in detail, above. As was noted, several tests are often employed (e.g., Sabin Feldman Dye test, IgM and IgA ELISAs, and the HS/AC differential agglutination test) to distinguish between an acute and chronic infection. Thus, there has been a need to develop an immunoassay which can accurately distinguish between an acute and chronic toxoplasmosis following an initial positive result for T. gondii antibodies. The present invention provides such an immunoassay. In particular, the present invention encompasses a recombinant Toxo P35 IgG immunoassay comprising a portion of the ToxoP35 protein (expressed, for example, in a prokaryotic cell such as E. coli), namely rPToxo-P35S (see FIG. 11), corresponding to amino acids 1-135 of P35 (see FIG. 11)(pJ0200-P35S), which detects Toxo IgG antibodies present in an acute infection and does not usually detect Toxo IgG antibodies present in a chronic infection. Thus, it is possible, using the Toxo P35 IgG immunoassay, to determine whether or not an acute toxoplasmosis has occurred during pregnancy. Results of such an immunoassay thereby facilitate an accurate diagnosis of the stage of infection which is important for the clinical management of both the mother and her fetus.

The present invention may be illustrated by the use of the following non-limiting examples:

### EXAMPLE 1

### General Methodology

Materials and Sources

Restriction enzymes, T4 DNA ligase, calf intestinal alkaline phosphatase (CIAP), polynucleotide kinase, and the Klenow fragment of DNA Polymerase I were purchased from New England Biolabs, Inc. (Beverly, Mass.) or from Boehringer Mannheim Corp. (Indianapolis, Ind.). DnaseI and aprotinin were purchased from Boehringer Mannheim Corp.

DNA and protein molecular weight standards, Daiichi pre-cast gradient polyacrylamide gels were obtained from Integrated Separation Systems, Inc. (Natick, Mass.).

Isopropyl-β-D-thiogalactoside (IPTG), Triton X-100, 4-chloro-1-naphthol, and sodium dodecyl sulfate (SDS) were purchased from BioRad Laboratories (Richmond, Calif.).

Plasma from patients with an acute Toxoplasma infection was obtained from Antibody Systems, Inc., Bedford, Tex.

Horseradish peroxidase (HRPO)-labelled antibodies were purchased from Kirkegaard & Perry Laboratories, Inc. (Gaithersburg, Md.).

EPICURIAN Coli™ XL-1 BLUE (recA1 enda1 gyrA96 thi-1 hsdR17 supE44 relA1 lac [F′ proAB lacI^{q} ZDMi15 Tn10 (Tet^{r})]) supercompetent E. coli cells, a DNA isolation kit, a RNA isolation kit, a ZAP™-Cdna Gigapack II Gold Cloning kit, a picoBLUE Immunoscreening kit, and Duralose-UV™ membranes, and a ZAP™-Cdna Synthesis kit were obtained from Stratagene Cloning Systems, Inc. (La Jolla, Calif.).

A GeneAmp™ reagent kit and AmpliTaq™ DNA Polymerase were purchased from Perkin-Elmer Cetus (Norwalk, Conn.). Deoxynucleotide triphosphates used in general procedures were from the GeneAmp™ reagent kit.

Supported nitrocellulose membrane was purchased from Schleicher & Schuell (Keene, N. H.).

A nucleotide kit for DNA sequencing with Sequenase™ and 7-deaza-Dgtp and Sequenase version 2.0 DNA Polymerase were obtained from U.S. Biochemical Corp. (Cleveland, Ohio).

A Multiprime DNA labelling kit, alpha-³²P-Dctp, and a-³²P-Datp were purchased from Amersham Corp. (Arlington Heights, Ill.).

A PolyA⁺ Mrna purification kit was purchased from Pharmacia LKB Biotechnology, Inc. (Piscataway, N.J.).

Polygard Cartridge filters, pore size 10 u, were purchased from Millipore Corp., Bedford, Mass.

Luria Broth plates with ampicillin (Lbamp plates) were purchased from Micro Diagnostics, Inc. (Lombard, Ill.).

OPTI-MEM™ Medium, Iscove's Modified Dulbecco's Media, Hank's Balanced Salt Solution, fetal calf serum, phosphate-buffered saline, competent E. coli DH5-alpha (F⁻ Ø80dlacZDM15 D(lacZYA-arqF)U169 deoR recA1 enda1 phoA hsdR17(r_{K}⁻, m_{K}⁺) supE44 l⁻ thi-1 gyrA96 relA1), and ultraPURE agarose were purchased from GIBCO BRL, Inc. (Grand Island, N.Y.).

Bacto-Tryptone, Bacto-Yeast Extract, and Bacto-Agar were obtained from Difco Laboratories (Detroit, Mich.).

NZY Broth was purchased from Becton Dickinson Microbiology Systems (Cockeysville, Md.).

Salmon sperm DNA, lysozyme, ampicillin, N-lauroyl sarcosine, thimerosal, buffers, casein acid hydrolysate, TWEEN 20™ (polyoxyethylenesorbitan monolaurate), diethylpyrocarbonate (DEPC), phenylmethylsulfonylfluoride (PMSF), bovine serum albumin (BSA), urea, glycerol, EDTA, sodium deoxycholate, pyrimethamine, sulfamethoxazole, mouse monoclonal antibody isotyping kits, and inorganic salts were purchased from Sigma Chemical Co. (Saint Louis, Mo.).

OPD (0-phenylenediamine dihydrochloride) and PBS (phosphate buffered saline) was purchased from Abbott Laboratories (Abbott Park, Ill.).

Hydrogen Peroxide (H₂O₂) was purchased from Mallinkrodt (Paris, Ky.).

Methanol was purchased from EM Science (Gibbstown, N.J.).

Microtiter Maxisorp plates were purchased from NUNC, Inc. (Naperville, Ill.).

Media, Buffers and General Reagents

“Superbroth II” contained 11.25 g/L tryptone, 22.5 g/L yeast extract, 11.4 g/L potassium phosphate dibasic, 1.7 g/L potassium phosphate monobasic, 10 Ml/L glycerol, adjusted Ph to 7.2 with sodium hydroxide.

“Tris-buffered saline” or “TES” consisted of 20 Mm Tris, 500 Mm NaCl at Ph 7.5.

“Tris-buffered saline TWEEN 20™”, or “TBST” consisted of TBS plus 0.05% TWEEN 20.

“Rubazyme specimen dilution buffer” or “Rubazyme SDB” consisted of 100 Mm Tris at Ph 7.5 with 135 Mm NaCl, 10 Mm EDTA, 0.2% TWEEN 20™, 0.01% thimerosal and 4% bovine calf serum.

“Rubazyme conjugate diluent dilution buffer” consisted of 100 Mm Trisat Ph 7.5 with 135 Mm NaCl, 0.01% thimerosal and 10% bovine calf serum.

“Membrane blocking solution” consisted of 1% BSA, 1 casein acid hydrolysate, 0.05% Tween 20 in TBS.

“TE buffer” consisted of 10 Mm Tris and 1 Mm EDTA at Ph 8.0.

“TEM lysis buffer” consisted of 50 Mm Tris, 10 Mm EDTA and 20 Mm magnesium chloride at Ph 8.5.

“PTE buffer” consisted of 50 Mm Tris and 10 Mm EDTA at Ph 8.5.

Parasite, Cell, and Mouse Lines

The RH strain of T. gondii (ATCC 50174) and the HeLa S3 cell line (ATCC CCL 2.2) were obtained from the American Type Culture Collection, Rockville, Md. The TS4 strain of T. gondii was also available from the American Type Culture Collection and from other sources. The Swiss mouse strain CD1 was obtained from Charles River Laboratories, Wilmington, Mass. Parasites were maintained by serial passage in the peritoneal cavity of Swiss mice. Tachyzoites were collected from the peritoneal cavity and used to inoculate a primary suspension culture of HeLa S3 cells. This infected suspension culture was grown for 2-4 days at 37° C. in Iscove's Modified Dulbecco's Media and then used to inoculate a secondary suspension culture of uninfected HeLa S3 cells. This secondary infected suspension culture was grown for 2-4 days at 37° C. in OPTI-MEM Reduced Serum Medium and used as a source of tachyzoites for screening monoclonal antibodies and for the preparation of DNA, RNA, and total tachyzoite protein.

General Methods

All enzyme digestions of DNA were performed according to suppliers' instructions. At least 5 units of enzyme were used per microgram of DNA, and sufficient incubation time was allowed for complete digestion of DNA. Supplier protocols were followed for the various kits used in manipulation of DNA and RNA, for polymerase chain reaction (PCR) DNA synthesis and for DNA sequencing. Standard procedures were used for Western and Southern Blots, partial restriction enzyme digestion of Toxoplasma genomic DNA with Sau 3AI, construction of a Toxoplasma genomic library, miniprep and large scale preparation of plasmid DNA from E. coli, preparation of phage lysate DNA from E. coli cells infected with phage lambda, preparation of E. coli lysates for the absorption of anti-E. coli antibodies, phenol-chloroform extraction and ethanol precipitation of DNA, restriction analysis of DNA on agarose gels, purification of DNA fragments from agarose gels, filling the recessed 3′ termini created by digestion with restriction enzymes using the Klenow fragment of DNA Polymerase I, and ligation of DNA fragments with T4 DNA ligase. (Maniatis et al., Molecular Cloning: A Laboratory Manual, 2^{nd} ed. (Cold Spring Harbor Laboratory Press, New York, 1989)).

DNA fragments for cloning into plasmids that were generated by PCR amplification, were extracted with phenol-chloroform and precipitated with ethanol prior to restriction enzyme digestion of the PCR reaction mixture. oligonucleotides for PCR and DNA sequencing were synthesized on an Applied Biosystems Oligonucleotide Synthesizer, model 380B or 394, per the manufacturer's protocol.

Mouse monoclonal antibody directed against the CKS protein was obtained by immunization of mice with purified rpHCV-23 (CKS-BCD), described in International Application No. WO93/04088 by Dailey et al. The proteins used for immunization were approximately 90% pure as determined by SDS-PAGE. The procedure for the immunization of mice, cell fusion, screening and cloning of monoclonal antibodies, and characterization of monoclonal antibodies were as described in Published International Application No. WO92/08738 by Mehta et al.

### EXAMPLE 2

Isolation of Toxoplasma DNA, RNA, Protein and Synthesis of Cdna

A 10 L secondary suspension culture of HeLa cells infected with the RH strain of T. gondii was grown to a tachyzoite density of approximately 1×10⁷ per ml and filtered through a 10 m Millipore Polygard cartridge filter to remove HeLa cells from the tachyzoites. The tachyzoite filtrate obtained contained less than 1% HeLa cells. The tachyzoites were then concentrated by centrifugation, washed and resuspended in 1× Hank's Buffer. The tachyzoite concentrate was then pipetted dropwise into liquid nitrogen, and the frozen tachyzoite pellets were recovered and stored at −80° C. until further use. The tachyzoite pellets were converted to tachyzoite powder by grinding the pellets to a fine powder using a mortar and pestle chilled with dry ice and liquid nitrogen. The tachyzoite powder was subsequently used for the isolation of tachyzoite nucleic acid and protein as described below.

Step A: Isolation of Toxoplasma DNA

Total Toxoplasma DNA was isolated from the tachyzoite powder using the Stratagene DNA extraction kit. The tachyzoite powder was dissolved in Solution 2, and total DNA was isolated following the kit's protocol. After ethanol precipitation and resuspension of the DNA in TE buffer, undissolved DNA and contaminating polysaccharides were removed by centrifugation at 200,000× g for 1 hr.

Step B: Isolation of Toxoplasma RNA

Total Toxoplasma RNA was isolated from the tachyzoite powder using the Stratagene RNA isolation kit. The tachyzoite powder was dissolved in Solution D, and total RNA was isolated following the kit's protocol. After ethanol precipitation and resuspension of the RNA in DEPC-treated water, polyA⁺ RNA was selected with an oligo-Dt column using a Pharmacia Mrna isolation kit. The purified Mrna was concentrated by ethanol precipitation and stored in DEPC-treated water at −80° C. until further use.

Step C: Isolation of Total Toxoplasma Protein

Total Toxoplasma protein was isolated from the tachyzoite powder by dissolving the powder in SDS-PAGE loading buffer and boiling the sample for 5 min. The protein preparation was stored at −20° C. until further use.

Step D: Synthesis of Toxoplasma Cdna

Purified Toxoplasma Mrna was used as a template for the synthesis of Cdna using the Stratagene ZAP-Cdna Synthesis kit. The first strand was synthesized using Moloney-Murine Leukemia Virus Reverse Transcriptase and a 50 mer primer which included an Xho I restriction enzyme site and an poly-Dt tract. The reaction mix included the analog 5-methyl Dctp to protect the Cdna from restriction enzymes used in subsequent cloning steps. The second strand was synthesized using Rnase H and DNA polymerase I. The Cdna was then ethanol precipitated and resuspended in water and stored at −20° C. until further use as a template for PCR amplification and for construction of a Toxoplasma Cdna library.

### EXAMPLE 3

### Cloning Strategy for Genes Encoding Toxoplasma Antigens

The immune response that is generated by human patients with Toxoplasmosis is targeted against several T. gondii proteins and varies by individual and by the disease stage. Hence, a Toxoplasma immunoassay which is composed entirely of purified protein antigens will require more than one protein serological target to accurately detect serum antibody to T. gondii in a population of Toxoplasma infected individuals. In order to identify additional Toxoplasma antigens which are relevant for human diagnostic testing, a two-tiered cloning strategy for genes encoding Toxoplasma antigens was undertaken. The first-tier consisted of cloning known genes encoding Toxoplasma antigens, by using the published DNA sequences for these genes. The second-tier consisted of cloning novel, previously undescribed genes encoding Toxoplasma antigens, by using pooled human plasma from patients with toxoplasmosis to screen a Toxoplasma Cdna library. The genes cloned in the first tier were then used as DNA probes to screen the genes cloned in the second tier for uniqueness.

Step A: Cloning of Toxoplasma Genes Encoding Known Toxoplasma Antigens

The CKS expression vector Pjo200 described in U.S. patent application Ser. No. 08/742,619 of Maine and Chovan allows the fusion of recombinant proteins to the CMP-KDO synthetase (CKS) protein. The DNA gene sequence which encodes for the structural protein CKS (also known as the kdsB gene) is published in Goldman et al., J. Biol. Chem. 261:15831 (1986). The amino acid sequence of CKS includes 248 amino acid (aa) residues and is described in Goldman et al., supra. The Pjo200 vector contained DNA encoding the sequence of the first 240 amino acids from the original kdsB gene followed by an additional 20 amino acids encoded for by the polylinker DNA sequence, for a total of 260 amino acids.

Oligonucleotide primers for use in the PCR amplification of known genes encoding Toxoplasma antigens were designed based on published DNA sequences. Each pair of PCR primers were “tailed” with additional DNA sequences to include restriction enzyme sites for subsequent cloning into the Pjo200 CKS expression vector. PCR amplification of each Toxoplasma gene with the appropriate primers was carried out using the GeneAmp reagent kit and AmpliTaq DNA Polymerase purchased from Perkin-Elmer Cetus, Norwalk, Conn., following the kit's protocol. Approximately 20 ng of Toxoplasma Cdna prepared in Example 2D or 20 ng of Toxoplasma genomic DNA prepared in Example 2A (for P66 genomic clone only) was used in each reaction. The amplification cycles were 1 cycle of 95° C. for 120 sec., followed by 35 cycles of 95° C. for 60 sec., 55° C. for 60 sec., 72° C. for 120 sec., followed by 1 cycle at 72° C. for 300 sec., followed by a soak cycle at 4° C. The PCR products obtained from the amplification reaction were then digested with the appropriate restriction enzymes, purified on agarose gels, ligated into the Pjo200 vector cut with the appropriate restriction enzymes and transformed into the Epicurean Coli XL-1 Blue Supercompetent E. coli cells following the kit protocol. Correct clones were confirmed by DNA sequence analysis of the cloned Toxoplasma DNA. The DNA sequences of the oligonucleotide primers used for the PCR amplification of the following Toxoplasma genes are shown below and how they were cloned into the Pjo200 CKS vector:

### Toxo P22 (SAG2) Gene

### (Prince et al. (1990) Mol. Biochem. Parasitol 43, 97-106)

Sense Primer [SEQ ID NO:1]:

5′-CGCAGAATTCGATGTCCACCACCGAGACGCCAGCGCCCATTGA-3′

(EcoRI site is underlined)

Antisense Primer [SEQ ID NO:2]:

5′-CCCGGGATCCTTACACAAACGTGATCAACAAACCTGCGAGACC-3′

(BamH-I site is underlined)

Region Cloned: Nucleotides 260-739 of the Toxo P22 gene cloned into the EcoRI/BamH-I sites of Pjo200 to yield plasmid Pjo200-P22.

### Toxo P24 (GRAL) Gene

### (Cesbron-Delauw et al. (1989) Proc. Nat. Acad. Sci. 86, 7537-7541)

| | |
|---|---|
| Sense Primer: | [SEQ ID NO:3] |

5′-GGCCGAATTCGATGGCCGAAGGCGGCGACAACCAGT-3′
(EcoRI site is underlined)

| | |
|---|---|
| Antisense Primer: | [SEQ ID NO:4] |

5′-GCCCGGATCCTTACTCTCTCTCTCCTGTTAGGAACCCA-3′
(BamH-I site is underlined)

Region Cloned: Nucleotides 685-1183 of the Toxo P24 gene cloned into the EcoRI/BamH-I sites of Pjo200 to yield plasmid Pjo200-P24.

### Toxo P25 Gene

### (Johnson et al. (1991) Gene 99, 127-132)

| | |
|---|---|
| Sense Primer: | [SEQ ID NO:5] |

5′-GGCGAATTCGATGCAAGAGGAAATCAAAGAAGGGGTGGA-3′
(EcoRI site is underlined)

| | |
|---|---|
| Antisense Primer: | [SEQ ID NO:6] |

5′-CGCACTCTAGATCACCTCGGAGTCGAGCCCAAC-3′
(XbaI site is underlined)

Region Cloned: Nucleotides 7-288 of the Toxo P25 gene cloned into the EcoRI/XbaI sites of Pjo200 to yield plasmid Pjo200-P25.

### Toxo P28 (GRA2) Gene

### (Prince et al. (1989) Mol. Biochem. Parasitol. 34, 3-13)

| | |
|---|---|
| Sense Primer: | [SEQ ID NO:7] |

5′-GGCGAATTCGATGAGCGGTAAACCTCTTGATGAG-3′
(EcoRI site is underlined)

| | |
|---|---|
| Antisense Primer: | [SEQ ID NO:8] |

5′-CGCTAGGATCCTTACTGCGAAAAGTCTGGGAC-3′
(BamH-I site is underlined)

Region Cloned: Nucleotides 489-924 of the Toxo P28 gene cloned into the EcoRI/BamH-I sites of Pjo200 to yield plasmid Pjo200-P28.

Toxo P30 (SAG1) Gene (Burg et al. (1988) J. Immunol. 141, 3584-3591)

| | |
|---|---|
| Sense Primer: | [SEQ ID NO:9] |

5′-GGCGAATTCGATGCTTGTTGCCAATCAAGTTGTCACC-3′
(EcoRI site is underlined)

| | |
|---|---|
| Antisense Primer: | [SEQ ID NO:10] |

5′-CGCTAGGATCCTCACGCGACACAAGCTGCGA-3′
(BamH-I site is underlined)

Region Cloned: Nucleotides 464-1318 of the Toxo P30 gene cloned into the EcoRI/BamH-I sites of Pjo200 to yield plasmid Pjo200-P30.

### Toxo P35 Gene

### (Knapp et al. (1989) EPA 431541A2)

| | |
|---|---|
| Sense Primer: | [SEQ ID NO:11] |

5′-GACGGCGAATTCGATGAACGGTCCTTTGAGTTATC-3′
(EcoRI site is underlined)

| | |
|---|---|
| Antisense Primer: | [SEQ ID NO:12] |

5′-CGCTAGGATCCTTAATTCTGCGTCGTTACGGT-3′
(BamH-I site is underlined)

Region Cloned: Nucleotides 91-822 of the Toxo P35 gene cloned into the EcoRI/BamH-I sites of Pjo200 to yield plasmid Pjo200-P35.

### Toxo P35 Gene Subclone#l (1-135aa)

### (Knapp et al. (1989) EPO 431541A2)

| | |
|---|---|
| Sense Primer: | [SEQ ID NO:13] |

5′-GACGGCGAATTCGATGAACGGTCCTTTGAGTTATC-3′
(EcoRI site is underlined)

| | |
|---|---|
| Antisense Primer: | [SEQ ID NO:14]: |

5′-CGCTAGGATCCTCAATGGTGAACTGCCGGTATCTCC-3′
(BamH-I site is underlined)

Region Cloned: Nucleotides 91-495 of the Toxo P35 gene cloned into the EcoRI/BamH-I sites of Pjo200 to yield plasmid Pjo200-P35S.

### Toxo P41 (GRA4) Gene

### (Mevelec et al. (1992) Mol. Biochem. Parasitol. 56, 227-238)

| | |
|---|---|
| Sense Primer: | [SEQ ID NO:15] |

5′-GGCGAATTCGATGGGTGAGTGCAGCTTTGGTTCT-3′
(EcoRI site is underlined)

| | |
|---|---|
| Antisense Primer: | [SEQ ID NO:16] |

5′-CGCACTCTAGATCACTCTTTGCGCATTCTTTCCA-3′
(XbaI site is underlined)

Region Cloned: Nucleotides 133-1107 of the Toxo P41 gene cloned into EcoRI/XbaI sites of Pjo200 to yield plasmid Pjo200-P41.

### Toxo P54 (ROP2) Gene

### (Saavedra et al. (1991) J. Immunol. 147, 1975-1982)

| | |
|---|---|
| Sense Primer: | [SEQ ID NO:17] |

5′-GCCTGAATTCGATGCACGTACAGCAAGGCGCTGGCGTTGT-3′
(EcoRI site is underlined)

| | |
|---|---|
| Antisense Primer: | [SEQ ID NO:18] |

5′-CGCTAGGATCCTCAGAAGTCTCCATGGCTTGCAATGGGAGGA-3′
(Cloned as a blunt end)

Region Cloned: Nucleotides 85-1620 of the Toxo P54 gene cloned into the EcoRI/SmaI sites of Pjo200 to yield plasmid Pjo200-P54.

### Toxo P66 (ROP1) Gene

### (Knapp et al. (1989) EPA 431541A2)

### (Ossorio et al. (1992) Mol. Biochem. Parasitol. 50, 1-15.

| | |
|---|---|
| Sense Primer: | [SEQ ID NO:19] |

5′-GGCGAATTCGATGAGCCACAATGGAGTCCCCGCTTATCCA-3′
(EcoRI site is underlined)

| | |
|---|---|
| Antisense Primer: | [SEQ ID NO:20] |

5′-CGCTAGGATCCTTATTGCGATCCATCATCCTGCTCTCTTC-3′
(BamH-I site is underlined)

Region Cloned: Nucleotides 122-1330 of the Toxo P66 gene cloned into the EcoRI/BamH-I sites of Pjo200 to yield plasmid Pjo200-P66 using Toxoplasma Cdna as template. Nucleotides 122-1330 of the Toxo P66 gene cloned into the EcoRI/BamH-I sites of Pjo200 to yield plasmid Pjo200-P66g using Toxoplasma genomic DNA as template.

### Toxo P68 Gene

### (Knapp et al. (1989) EPA 431541A2)

| | |
|---|---|
| Sense Primer: | [SEQ ID NO:21] |

5′-ACCCGAATTCGATGACAGCAACCGTAGGATTGAGCCAA-3′
(EcoRI site is underlined)

| | |
|---|---|
| Antisense Primer: | [SEQ ID NO:22] |

5′-CGCTGGATCCTCAAGCTGCCTGTTCCGCTAAGAT-3′
(BamH-I site is underlined)

Region Cloned: Nucleotides 294-1580 of the Toxo P68 gene cloned into the EcoRI/BamH-I sites of Pjo200 to yield plasmid Pjo200-P68.

Step B: Construction and Immunoscreening of a Toxoplasma Cdna Library

A Toxoplasma Cdna library was constructed in the UNIZAP XR vector using the Stratagene ZAP-Cdna Synthesis kit and ZAP-Cdna Gigapack II Gold Cloning kit. The Cdna produced in Example 2D was further processed using the kit protocols as briefly outlined below. The Cdna ends were blunted with T4 DNA polymerase, and EcoRI restriction site adapters were ligated to the blunt-ended Cdna. The RI adaptors ligated to the Cdna were then kinased with T4 Polynucleotide Kinase. The Cdna was digested with the restriction enzymes EcoRI and XhoI and then ligated to the phage lambda UNIZAP XR vector arms. The Cdna is cloned unidirectionally into this vector, resulting in the 5′ end of the Cdna located downstream of the lacZ gene. If the coding sequence of the Cdna is in frame with the lacZ gene, a lacZ-Toxo fusion protein will be expressed. The UNIZAP XR-Toxo Cdna ligation mixture was packaged into phage in vitro, and a primary Toxoplasma Cdna phage library was obtained with 660,000 members. This library was amplified and checked for the size and frequency of the cloned Cdna inserts by converting a dozen random phage clones to E. coli phagemid (plasmid) clones using the Stratagene in vivo subcloning protocol from the ZAP-Cdna Synthesis kit. This procedure excises the cloned Cdna insert and the Pbluescript plasmid from the phage resulting in a Pbluescript plasmid clone containing the cloned Cdna. Miniprep DNA was made from the phagemid clones and analyzed with restriction enzymes on DNA agarose gels. Greater than 90% of the phagemid clones contained insert DNA with an average size of 0.8 Kb. This library was used for immunological screening with pooled plasma obtained from patients with Toxoplasmosis as described below.

Plasmas obtained from individuals in the acute phase of Toxoplasmosis infection were pooled. Samples used for this pool were tested by the Abbott Imx Toxo IgM and Toxo IgG immunoassays (Abbott Laboratories, Abbott Park, Ill.), and only samples that contained IgM antibodies and no detectable levels of IgG antibody were pooled. Prior to immunoscreening, the pooled plasma was treated to remove E. coli cross-reactive antibodies. The procedure followed was a modification of the protocol described in the Stratagene picoBLUE immunoscreening kit. Pooled plasma was initially diluted 1:5 in Rubazyme specimen dilution buffer and E. coli cross-reactive antibodies were removed by incubating the diluted pool plasma with several nitrocellulose filters coated with E. coli lysate as described in the kit protocol. After absorption of E. coli antibodies, the plasma pool was stored at 4° C. until further use.

The Toxoplasma Cdna library was immunologically screened following a modification of the Stratagene picoBLUE Immunoscreening kit protocol. Briefly, recombinant phage absorbed to the XL-1 Blue strain of E. coli were plated onto pre-warmed 150 mm NZY plates at a density of 20,000 phage per plate and incubated for 3.5 hrs. at 42° C. Duralose UV membranes pretreated with 10 Mm IPTG and dried were then overlayed on each plate and incubated for an additional 4 hrs. at 37° C. The filters were oriented by piercing them with an 18 gauge needle, removed from the plate and washed 3× with TBST buffer at room temperature, 10 min. per wash. The filters were then washed once for 10 min. with TBS buffer at room temperature and blocked overnight at 4° C. in membrane blocking solution. The next day the filters were incubated for 2 hrs. at room temperature with the acute phase plasma pool (at 1:40 dilution in Rubazyme SDB). The filters were then washed 2× with TBST for 10 min. per wash and once with TBS for 10 min. and then incubated for 1 hr. at room temperature with goat anti-Human IgM (H+L) horseradish peroxidase-labelled antibody. The filters were washed again as before and developed for 10 min. in HRP color development solution. The filters were then extensively washed with tap water to stop the color development reaction, and plaques which gave a strong blue color were subsequently plaque purified twice and retested for immunoreactivity against the appropriate pool of plasma. Approximately 130,000 plaques were screened with the pooled acute phase plasma with the isolation of 4 positive clones. These phage clones were converted to plasmid clones using the Stratagene in vivo subcloning protocol from the ZAP-Cdna Synthesis Kit and further characterized as described below.

Step C: Characterization of the Immunopositive Clones Isolated with the Acute Phase Plasma Pool

The 4 immunopositive clones isolated with the acute phase plasma pool were designated Pgm610, Pgm611, Pgm612, and Pgm613 and were analyzed with restriction enzymes on DNA agarose gels. Clones Pgm610 and Pgm612 contained a 1.1 Kb insert of DNA, clone Pgm611 contained a 0.7 Kb insert of DNA, and clone Pgm613 contained a 1.3 Kb insert of DNA. The Cdna inserts contained in these clones were removed from the Pbluescript vector by restriction enzyme digestion and purified on DNA agarose gels. These 4 purified Cdna inserts were individually labelled with alpha-³²P-Dctp using the Multiprime DNA labelling kit and protocol from Amersham for hybridization to colony filters and genomic Toxoplasma DNA. Filters for colony hybridization were prepared by gridding E. coli clones containing the cloned Toxoplasma genes described in Examples 3A and 3B onto Duralose UV membranes overlaid on Lbamp plates. These plates were grown overnite at 37° C., and the next day the E. coli colonies were lysed with alkali and prepared for DNA colony hybridization as described in GENERAL METHODS. After hybridization and washing, the hybridization signal was visualized by autoradiography with the result that all 4 immunopositive clones were homologous to one another and are non-homologous to the other 10 genes tested (see Example 3A). In order to determine the homology between the immunopositive clones and between Toxoplasma genomic DNA, the following Southern blot experiment was performed as described in GENERAL METHODS. Toxoplasma genomic DNA and two of the immunopositive clones were digested with restriction enzymes, run on DNA agarose gels, transferred to nitrocellulose and probed with purified radioactively-labelled Cdna inserts from clones Pgm611 and Pgm613. After hybridization and washing, the hybridization signal was visualized by autoradiography with the result that both clones were homologous to one another and all hybridized to the genomic blot of Toxoplasma DNA. Therefore, these 4 immunopositive clones contained the same Toxoplasma gene encoding a novel antigen which was designated Pₙₒᵥₑₗ₂.

### EXAMPLE 4

Construction of CKS-Pₙₒᵥₑₗ₂ Expression Vector Based on Pjo200

The gene encoding the Pₙₒᵥₑₗ₂ antigen was subcloned into the Pjo200 vector in order to produce adequate levels of fusion protein for further analysis. Since the reading frame of the lacZ gene in the Pbluescript vector and the reading frame of the CKS gene in the Pjo200 vector are the same, presence of the EcoRI site at the juncture of the CKS and Toxoplasma genes ensured that the Toxoplasma gene was fused translationally in frame with the CKS gene. In order to remove the Cdna insert from the Pbluescript vector and subclone it into the Pjo200 vector, the following digests were performed:

The CKS expression vector Pjo200 described in Example 3A was digested with EcoRI and SmaI and the vector backbone was purified on an agarose gel in preparation for subcloning. Plasmid DNA from the largest Pₙₒᵥₑₗ₂ clone Pgm613 was digested with Asp718 and then treated with the Klenow fragment of DNA Polymerase I to render the ends blunt-ended. Subsequently, the DNA was extracted and then digested with EcoRI, and the 1.3 Kb EcoRI/Asp718 (Klenow) DNA fragment from Pgm613 was purified on an agarose gel and ligated to Pjo200/EcoRI/SmaI overnight at 16° C.

The next day, the ligation mixture was transformed into competent XL-1 Blue cells. Miniprep DNA was prepared from the transformants and screened for the presence of the 1.3 Kb DNA fragment inserted at the EcoRI/SmaI sites of Pjo200. The correct CKS-Pₙₒᵥₑₗ₂ clone identified by restriction analysis was designated Pjo200-Pₙₒᵥₑₗ₂.

### EXAMPLE 5

Expression of Recombinant Toxo Antigens and CKS in E. coli

Step A: Expression of cloned genes in E. coli

Bacterial clones Pjo200-P22, Pjo200-P24, Pjo200-P25, Pjo200-P28, Pjo200-P30, Pjo200-P35S, Pjo200-P41, Pjo200-66g, Pjo200-68 and Pjo200-Pₙₒᵥₑₗ₂ expressing the CKS fusion proteins rpJO200-P22, rpJO200-P24, rpJO200-P25, rpJO200-P28, rpJO200-P30, rpJO200-P35S, rpJO200-P41, rpJO200-66g, RpjO200-68 and rpJO200-Pₙₒᵥₑₗ₂ of Examples 3 and 4 and the control bacterial strain expressing unfused CKS were grown in “SUPERBROTH II” media containing 100 ug/ml ampicillin to log phase, and the synthesis of the CKS-Toxo fusion protein and unfused CKS was induced by the addition of IPTG as previously described (Robinson et al. (1993) J. Clin. Micro. 31, 629-635). After 4 hours post-induction, the cells were harvested, and the cell pellets were stored at −80° C. until protein purification occurred.

Step B: Purification of Recombinant Toxo Antigens and CKS Protein

Insoluble recombinant antigens rpJO200-P22, rpJO200-P25, rpJO200-P30, rpJO200-P35S, rpJO200-P41, rpJO200-66g, and rpJO200-Pₙₒᵥₑₗ₂ were purified after lysis from cell paste by a combination of detergent washes followed by solubilization in 8M urea (Robinson et al. (1993) J. Clin. Micro. 31, 629-635). After solubilization was complete, these proteins were filtered through a 0.2 u filter and further purified by chromatography on Sephacryl S-300 columns. The appropriate column fractions were pooled for each protein and stored at 2-8° C. for evaluation by microtiter ELISA. Soluble rpJO200-P24, rpJO200-P28, rpJO200-P68, and unfused CKS proteins were purified after cell lysis by ammonium sulfate precipitation followed by ion-exchange chromatography. The appropriate column fractions were pooled for each protein, dialyzed against the appropriate buffer, and stored at 2-8° C. for evaluation by microtiter ELISA.

### EXAMPLE 6

Evaluation of Human Sera with the Recombinant Toxo Antigens in Microtiter ELISA

Step A: Human Sera for Testing

The tests used for determining the presence of IgG and IgM antibody in sera were the Abbott Toxo-G and Toxo-M MEIA assays, respectively. Twenty-four Toxo IgG positive sera, eighteen Toxo IgM positive sera, and nineteen sera negative for Toxo IgG and IgM antibody were evaluated using the recombinant Toxo antigens in Microtiter ELISA.

Step B: Evaluation of Human Sera in the Recombinant Toxo Antigen Microtiter ELISA

Purified recombinant Toxo antigens (Example 5B) were individually diluted to 5.0 ug per ml in PBS, and 0.1 ml of each antigen was added to separate wells of microtiter Maxisorp plates. Control wells for each sera were coated with E. coli lysate at 5.0 ug per ml. Plates were incubated at 37° C. for 1 hr and stored overnight at 4° C. The next day, the plates were washed three times with distilled water and blocked for 2 hrs at 37° C. with 0.2 ml of blocking solution (3% fish gelatin, 10% fetal calf serum in PBS, 0.22 u). The plates were then washed three times with distilled water and ready for incubation with serum. Each serum specimen was tested in duplicate with each antigen at a 1:200 dilution into Rubazyme SDB containing 2% E. coli lysate. After adding 0.1 ml of diluted specimen to each well, the plates were incubated for 1 hr. at 37° C. The plates were then washed three times with PBS-Tween and three times with distilled water. Bound human IgG and IgM were detected by using goat anti-human IgG-HRPO and IgM-HRPO conjugates, respectively, diluted 1:1,000 in Rubazyme conjugate diluent buffer and filtered. After addition of 0.1 ml of the appropriate diluted conjugate, the plates were incubated for 1 hr. at 37° C. and washed three times with PBS-Tween and three times with distilled water. The OPD color development reagent was prepared per manufacturer's directions and 0.1 ml was added to each well. After 2 minutes, the color development reaction was stopped by adding 0.1 ml of 1N sulfuric acid, and the plate was read in a microtiter plate reader. The net OD was obtained by subtracting the OD for the E. coli lysate control from that of the test with each recombinant antigen. The cut-off for these assays was between 2 to 3 standard deviations from the mean of the negative population for each antigen.

The results of the evaluation of human sera in the recombinant microtiter ELISA are shown in Table 1 for detection of Toxoplasma-specific IgG antibody and in Table 2 for detection of Toxoplasma-specific IgM antibody. The performance of each antigen was ranked in decreasing order of the antigen with the largest number of positive specimen results per total number of positive (IgM or IgG) specimens tested.

TABLE 1
Relative rank of Antigen Performance in
Microtiter IgG ELISA

Immunoreactivity

| | IgG⁻ | IgG⁺ |
|---|---|---|
| | # Pos Results/ | # Pos Results/ |
| | Total # IgG − | Total # IgG + |
| Antigen | Specimens Tested | Specimens Tested |
| P68 | 1/19 | 16/24 |
| P35S | 1/19 | 14/24 |
| P24 | 0/19 | 14/24 |
| P30 | 2/18 | 13/24 |
| Pnovel2 (P29) | 1/19 | 13/24 |
| P22 | 0/19 | 13/24 |
| P30 | 2/18 | 13/24 |
| P41 | 0/19 | 10/24 |
| P25 | 1/19 | 10/24 |
| P28 | 1/19 | 10/24 |
| P66 | 2/19 | 9/24 |

TABLE 2
Relative rank of Antigen Performance in
Microtiter IgM ELISA

Immunoreactivity

| | IgM⁻ | IgM⁺ |
|---|---|---|
| | # Pos Results/ | # Pos Results/ |
| | Total # IgM − | Total # IgM + |
| Antigen | Specimens | Specimens |
| P66 | 1/18 | 17/18 |
| P35 (1-135) | 0/18 | 15/18 |
| Pnovel2 (P29) | 0/19 | 10/18 |
| P68 | 0/19 | 5/18 |
| P22 | 0/19 | 5/18 |
| P28 | 1/18 | 4/18 |
| P41 | 0/18 | 3/18 |
| P25 | 0/19 | 3/18 |
| P30 | 1/18 | 2/18 |
| P24 | 1/19 | 0/18 |

As can be seen from Table 1, there was no single recombinant Toxo antigen capable of detecting as positive all 24 IgG positive specimens. Hence, an immunoassay employing some combination of the antigens listed in Table 1 is required to detect all the IgG positive specimens.

As can be seen from Table 2, there was no single recombinant Toxo antigen capable of detecting as positive all 18 IgM positive specimens. Hence, an immunoassay employing some combination of the antigens listed in Table 2 is required to detect all the IgM positive specimens.

### EXAMPLE 7

Generation of a Monoclonal Antibody Reactive With CKS-Pₙₒᵥₑₗ₂ Antigen

Step A: Immune Response Study in Mice and Generation of Hybridomas

Animals, including mice, rats, hamsters, rabbits, goats and sheep may be infected with a lethal dose of tachyzoites, rescued from death with drug therapy and later used for hybridoma development. There are two hydbridoma development advantages for using this process that otherwise would not be possible. The first advantage is that time is allowed for a diverse repertoire of antibodies to be generated against native T. gondii (or Borrelia burgdorferi, Schistosoma sp., for example, Schistosoma treponema, or sporozoans other than T. gondii, for example, members of the genus Plasmodium (e.g., P. vivax and P. falciparum) and other possible members of the genus Toxoplasma)), and the second advantage is that the rescue allows time for affinity maturation of the immune response.

In the present experiment, Swiss mice were infected intraperitonally with 2.5×10⁷ tachyozoites of T. gondii strain TS4. Five days later mice were treated orally with 10 mg pyrimethamine and 200 mg sulfamethoxazole per kg daily for 10 days. (This technique can be repeated every 6-8 weeks if desired.) After 12 additional weeks, these mice were injected intravenously with 1.2×10⁷ sonicated tachyzoites 3 days prior to fusion to minimize the biohazardous status. One hundred percent of the mice survived (providing evidence of a humane method). Resulting hybrids from the PEG mediated fusion of splenocytes and the SP2/0 myeloma were screened on the sonicated tachyzoites and CKS-Pₙₒᵥₑₗ₂ antigen (Kohler, G. and Milstein, C. (1975) Nature 256 495-497; Kohler, G. and Milstein, C. (1976) Eur. J. Immunol. 6, 511-519; Goding, J. (1986) Monoclonal Antibodies: Principles and Practice. 2^{nd} Ed. Academic Press London).

It should also be noted that monoclonal antibodies may be produced by immunizing mice by intraperitoneal infection with T. gondii (Mineo et al. (1993) J. Immunol. 150, 3951-3964;Handman et al. (1980) J. Immunol. 124, 2578-2583; Grimwood and Smith (1992) Exp. Parasitol. 74, 106-111) or with fractions of T. gondii (Prince et al. (1990) Mol. Biochem. Parasitol. 43, 97-106). Fusion of spleen cells and myeloma cells may then be carried out directly, subsequent to immunization, without a drug therapy step (see, e.g., Kohler and Milstein, supra (1975)).

Step B: Screening and Isolation of a Monoclonal Antibody to rpCKS-Pₙₒᵥₑₗ₂

Bacterial clone Pjo200-Pₙₒᵥₑₗ₂ expressing the CKS-Pₙₒᵥₑₗ₂ fusion protein of Example 4 (rpJO200-Pₙₒᵥₑₗ₂) and the control bacterial strain expressing unfused CKS were grown in Superbroth II media containing 100 ug/ml ampicillin to log phase, and the synthesis of the CKS-Toxo fusion protein and unfused CKS was induced by the addition of IPTG as previously described in Example 5A. In preparation for screening hybridoma fluids obtained in Example 7A, cell pellets were thawed, resuspended in 10 ml of PBS and sonicated for 0.5 min in an icewater bath. The antigen preparation was diluted 1:40 in 0.05 M sodium carbonate-bicarbonate, Ph 9.6, containing 15 Mm sodium azide after which 0.1 ml of this suspension was placed in wells of NUNC Maxisorb microtiter plates. When tachyzoites were tested, 3×10⁶ sonicated tachyzoites were added to wells. Plates were incubated at 37° C. for 1 hr, stored 1 to 3 days at 4° C., and washed three times with distilled water. Hybridoma fluids obtained in Example 7A were diluted 1:10 in Rubazyme SDB. The remainder of the ELISA was performed as described above in Example 6B except bound antibody was detected by mixture of horseradish peroxidase-conjugated goat anti-mouse IgG and IgM, each diluted to 1.0 ug per ml in Rubazyme conjugate diluent buffer.

Positive hybridoma clones were cloned by limiting dilution, and hybridoma fluid was retested by microtiter ELISA containing rpJO200-Pₙₒᵥₑₗ₂, unfused CKS, and sonicated tachyzoites. One highly reactive monoclonal antibody clone was isolated which was designated Toxo Mab 5-241-178, which reacted very strongly with sonicated tachyzoites and rpJO200-Pₙₒᵥₑₗ₂ but showed no reactivity to unfused CKS. This hybridoma clone was found to produce IgG type antibodies as determined using a mouse monoclonal antibody isotyping kit from Sigma.

Step C: Identification of the Pₙₒᵥₑₗ₂ Gene Encoding the Toxoplasma P29 Antigen Using Toxo Mab 5-241-178

Total Toxoplasma protein prepared as described in Example 2C was loaded onto an 4-20% gradient Daiichi SDS-PAGE gel along with protein standard molecular weight markers, and transferred to nitrocellulose as described in General Methods. The Western blot was probed with the Toxo Mab 5-241-178 antibody, and the blot was visualized with a goat anti-mouse IgG-HRPO conjugate followed by BioRad Color Development Reagent (4-chloro-1-naphthol and hydrogen peroxide) per manufacturer's directions. A single protein band of 29,000 molecular weight from the Toxoplasma protein prepared from tachyzoites was immunoreactive with the Toxo Mab 5-241-178 indicating that the Pₙₒᵥₑₗ₂ gene cloned in plasmid Pgm613 (Example 3C) and Pjo200-Pₙₒᵥₑₗ₂ (Example 4) encodes the P29 antigen of Toxoplasma.

### EXAMPLE 8

DNA Sequence of Clone Pgm613 and Deduced Amino Acid Sequence

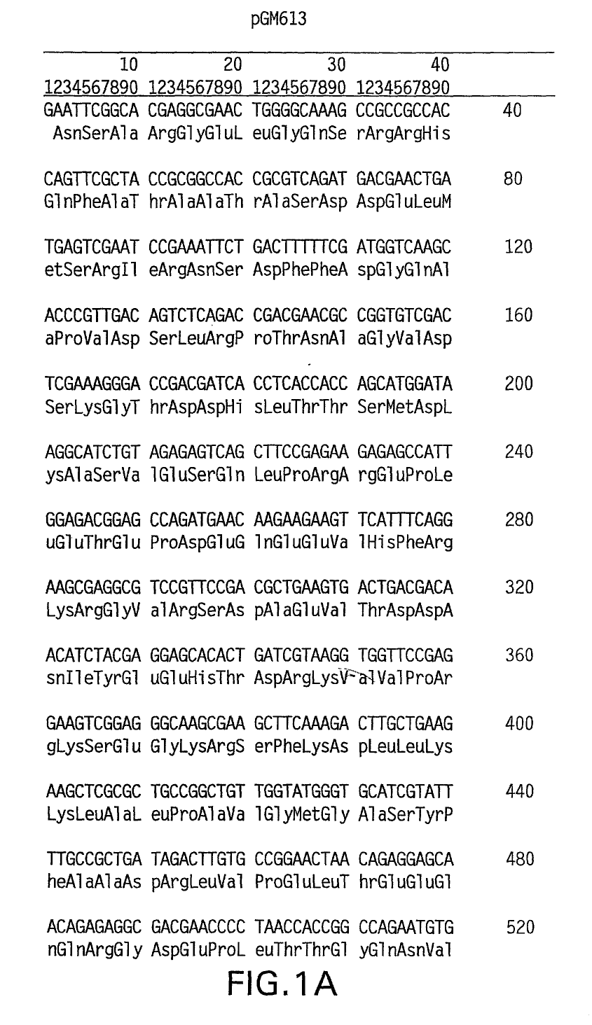

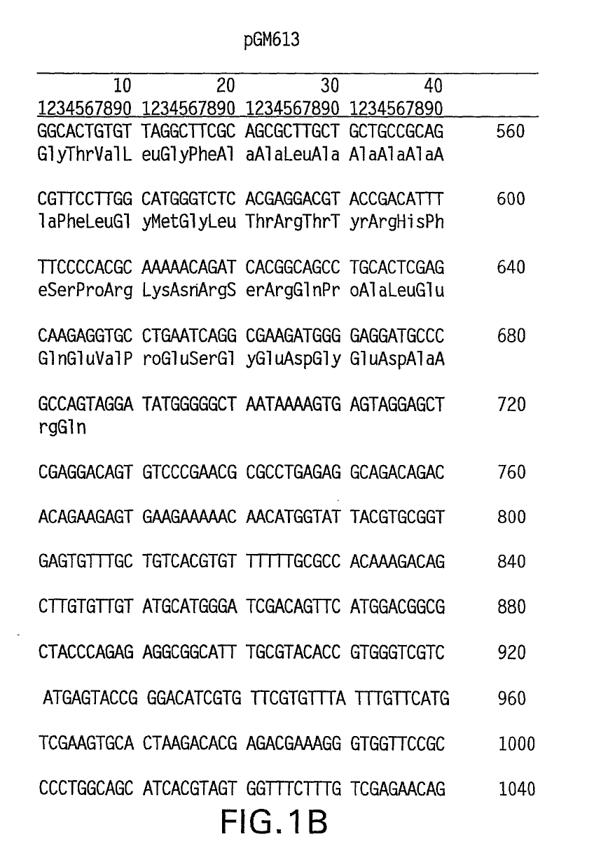

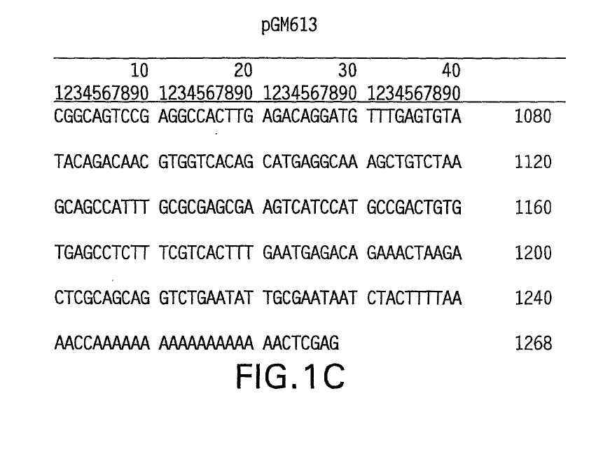

The 1.3 Kb EcoRI/XhoI insert of Toxoplasma Cdna contained in Pgm613 was sequenced as described in General Methods. The DNA sequence (1268 bp) [SEQ ID NO:23] and the deduced amino acid sequence (228 aa) [SEQ ID NO:24] in-frame with the lacZ gene are shown in FIG. 1. The open reading frame (nucleotide position 2 to 685) present in this sequence can code for a protein of approximately 25,000 molecular weight. The first ATG present in the DNA sequence is located at nucleotide position 80 and is not surrounded by sequences fulfilling the criteria for initiation of translation (Kozak, M. (1986) Cell 44, 283-292) and is probably not the initiator methionine residue. Hence, it is likely that the insert of Toxoplasma Cdna present in clone Pgm613 is not full-length.

Genebank's non-redundant protein, DNA, and dbEST/dbSTS sequences (tags) database and the Derwent DNA and protein patent databases were searched for homology to the DNA sequence and the deduced amino acid sequence of clone Pgm613.Homology of DNA sequence and the deduced amino acid sequence was found between a portion of the Pgm613 clone (nucleotide positions 461-684, amino acid residues 153-228) and the F29 clone of Knapp et al. contained in European Patent Application 0431541A2. In addition, homology between the DNA sequence of Pgm613 and several T. gondii expressed sequence tags of unknown function isolated by Wan, K. -L. et al. (1996) Molec. And Biochem. Parasitol. 75, 179-186 was also found.

### EXAMPLE 9

Isolation and Characterization of a Genomic Clone Containing the P29 Gene and Generation of a Composite DNA Sequence

Since the Cdna insert of Pgm613 encoding the P29 antigen of Toxoplasma appeared to be less than full-length, a portion of the Pgm613 Cdna sequence was used as a probe to isolate a genomic clone of the P29 antigen with the goal of cloning the remaining 5′ end of the gene.

Step A: Construction of a Toxoplasma Genomic DNA Library in Pjo200

A Toxoplasma genomic DNA library was constructed in the Pjo200 vector as follows. Toxoplasma genomic DNA prepared in Example 2A was treated by a partial digestion with the restriction enzyme Sau 3AI as described in General Methods. The partially digested genomic DNA was subsequently electrophoresed on a 0.7% agarose gel with molecular weight standards and the 6-15 Kb molecular weight range of the DNA was isolated, purified, and extracted as described in General Methods. In preparation for ligation with the genomic DNA, plasmid Pjo200 was digested with BamH-I followed by dephosphorylation with the CIAP enzyme. The resulting vector backbone was extracted and then ligated overnight at 16° C. with the Sau 3AI digested DNA. The ligation mixture was transformed the next day into competent XL-1 Blue cells, and the resulting transformants were pooled resulting in a primary Toxoplasma genomic library containing 80,000 members.

Step B: Screening Toxoplasma Genomic Library With P29 5′ Gene Probe

In order to isolate the 5′ end of the P29 gene from the genomic library, a portion of the 5′ end of the Cdna clone present in Pgm613 was selected as a probe. This portion of the Cdna was then used to probe the Toxoplasma genomic library prepared in Example 9A for genomic clones homologous to the 5′ end of the Cdna.

Plasmid Pgm613 was digested with SacII and HindIII, and the 326 bp SacII/HindIII fragment containing the 5′ end of the Cdna insert in Pgm613 (nucleotide positions 55-380, see FIG. 1) was gel purified. This gene fragment was radioactively labelled and used to probe the Toxoplasma genomic library by colony hybridization as described in General Methods. Positive clones obtained by hybridization were colony purified and retested. One positive clone designated Ptxg1-2 containing a 6.5 Kb insert of DNA was further characterized as described below.

Step C: DNA Sequence of Genomic Clone Ptxq1-2 and Composite DNA Sequence for the P29 Gene and the Deduced Amino Acid Sequence

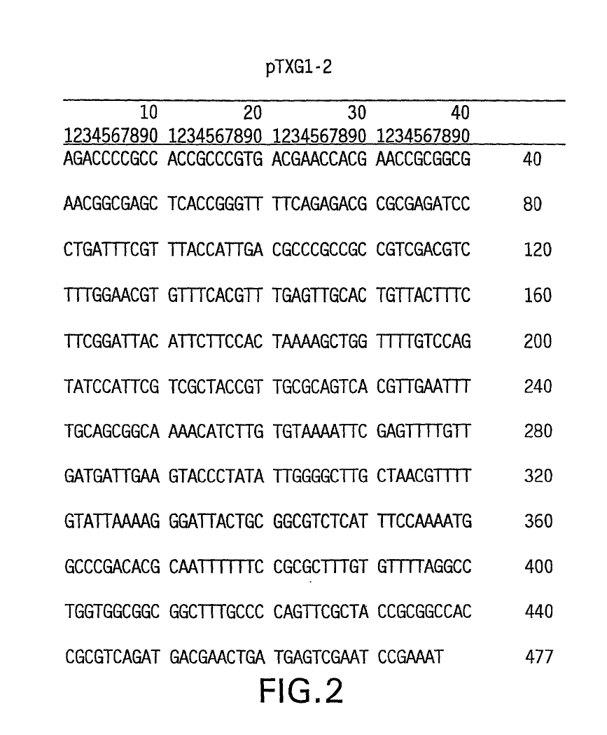

The 5′ end of the P29 gene contained in clone Ptxg1-2 was sequenced as described in General Methods using DNA primers complementary to the 5′ end of the Cdna contained in clone Pgm613. The DNA sequence obtained for clone Ptxg1-2 [[SEQ ID NO:25] is shown in FIG. 2. An alignment of the DNA sequences for genomic clone Ptxg-1 and the Cdna clone Pgm613 was then performed resulting in the composite DNA sequence [SEQ ID NO:26] and deduced amino acid sequence [SEQ ID NO:27] for the P29 gene as shown in FIG. 3. The composite DNA sequence is derived from the genomic sequence of clone Ptxg-1 (FIG. 2, [SEQ ID NO:25]) and the Cdna sequence of Pgm613 (FIG. 1, [SEQ ID NO:23]) as shown below in Table 3.

TABLE 3
Source of Sequence for the Composite DNA Sequence for
the P29 Gene

| Nucleotide | Nucleotide | Nucleotide |
|---|---|---|
| Position | Position | Position |
| Composite | Genomic | Cdna |
| Sequence | Sequence | Sequence |
| 1-419 | 1-419 | None |
| 420-477 | 420-477 | 40-97 |
| 478-1648 | None | 98-1268 |

The only good candidate for the initiator methionine residue for the start of translation of the P29 gene is the first methionine shown on FIG. 3 starting at nucleotide position 358. This is the only methionine in-frame with the reading frame present in the Cdna clone Pgm613. If the same reading frame is examined further upstream of the methionine at position 358, no further methionine residues are found before an in-frame UAA stop codon present at position 325. The methionine at nucleotide position 358 is surrounded by sequences fulfilling the criteria for initiation of translation (Kozak, M. (1986) Cell 44, 283-292) and is followed by amino acid residues that constitute a signal peptide (von Heijne, G. (1986) Nucleic Acids Res. 14, 4683-4690).

### EXAMPLE 10

### Construction of an Improved CKS Epitope-embedding Vector Pee3

The CKS epitope-embedding expression vector Peel described in U.S. patent application Ser. No. 08/742,619 of Maine and Chovan allows for the embedded fusion of recombinant proteins to the CMP-KDO synthetase (CKS) protein. In order to facilitate the cloning of the P29 gene into the CKS epitope-embedding vector, the Peel vector was modified in two steps. First, an obsolete polylinker near the 3′ end of the CKS gene in the Peel vector was removed generating an intermediate vector Pee2. Secondly, a new polylinker was introduced into the coding region of CKS, thus permitting the embedding of genes using a variety of restriction sites (StuI, EcoRI, SacI, BamH-I, PstI, MluI) into the CKS gene.

Step A: Construction of Pee2

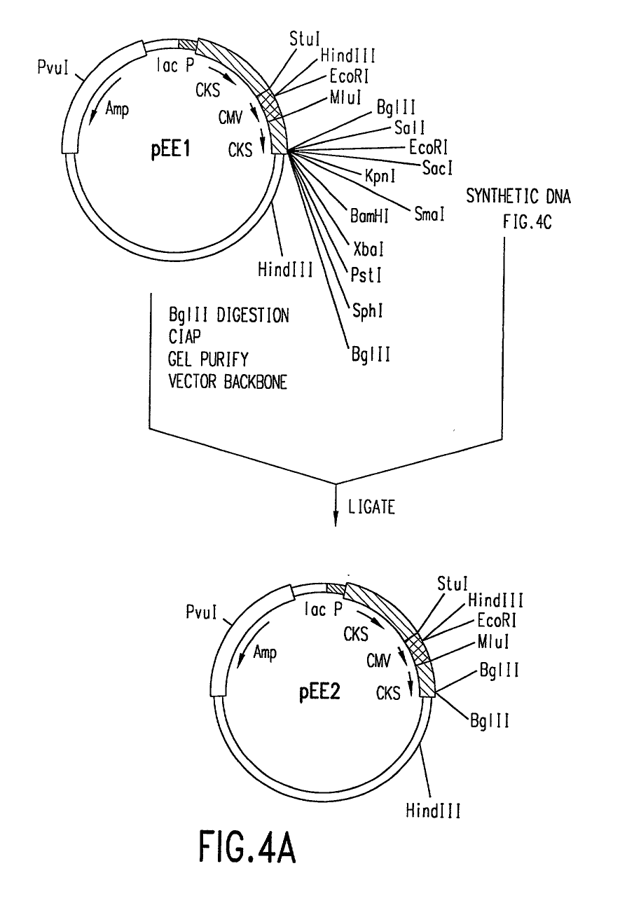

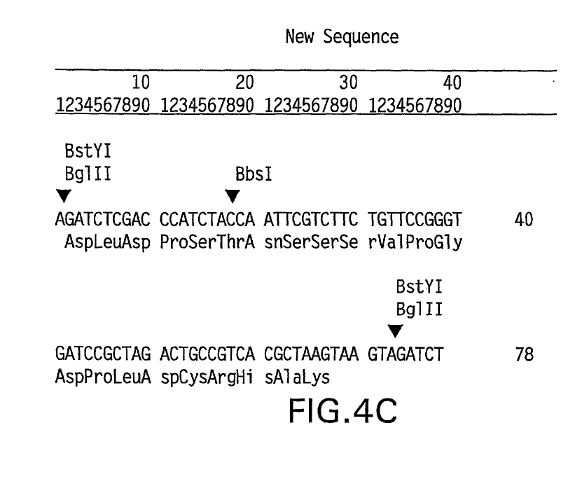

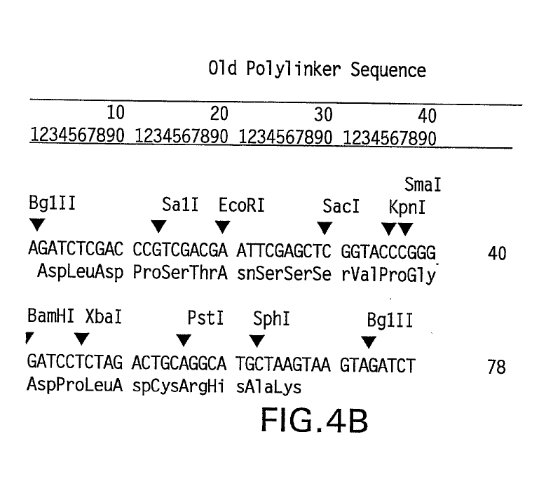

The plasmid Pee2, a derivative of the CKS expression vector Peel (FIG. 4A), was constructed by digesting Peel with the Bgl II restriction enzyme and removing a polylinker located at the 3′ end of the CKS gene which had the sequence (5′-3′) [SEQ ID NO:28] (FIG. 4B) and the deduced amino acid sequence [SEQ ID NO:49]

AGATCTCGACCCCTCCACCAATTCGAGCTCGGTACCCGGGGATCCTCTAGAC
AspLeuAspProSerThrAsnSerSerSerValProGlyAspProLeuAsp
TGCAGGCATCCTAAGTAAGTAGATCT
CysArgHisAlaLys

and replacing it with the following sequence (5′-3′) [SEQ ID NO:29] (see FIG. 4C) and the deduced amino acid sequence [SEQ ID NO:50]

AGATCTCGACCCATCTACCAATTCGTCTTCTGTTCCGGGTGATCCGCTAGAC
AspLeuAspProSerThrAsnSerSerSerValProGlyAspProLeuAsp
TGCCGTCACGCTAAGTAAGTAGATCT
CysArgHisAlaLys

As shown in FIGS. 4B and 4C, this sequence replacement removes the restriction sites SalI, EcoRI, SacI, KpnI, SmaI, BamH-I, XbaI, PstI, and SphI, thus enabling the use of these sites in a new polylinker to be embedded later within the CKS gene further upstream (Example 10B).

Plasmid Peel was digested with Bgl II and then treated with the CIAP en yme to remove the five prime phosphate groups to prevent self-ligation. The Peel/Bgl II dephoshorylated vector backbone was then purified on an agarose gel. Two oligonucleotides shown below (5′-3′) were synthesized for ligation into the Peel/Bgl II backbone.

[SEQ ID NO:30]
CCTGAAGATCTCGACCCATCTACCAATTCGTCTTCTGTTCCGGGTGATCC
GCTAGACTGCCGTCACGCTAAGTAAGTAGATCTTGACT
[SEQ ID NO:31]
AGTCAAGATCTACTTACTTAGCGTGACGGCAGTCTAGCGGATCACCCGGA
ACACAAGACGAATTGGTAGATGGGTCGAGATCTTCAGG

These oligonucleotides were mixed together, heated to 85° C. and then allowed to cool gradually overnight to 4° C. to permit annealing of the oligonucleotides. The annealed oligonucleotides were then digested with the Bgl II enzyme, extracted, and then ligated to the Peel/Bgl II backbone overnight at 16° C. The ligation mixture was transformed the next day into competent XL-1 Blue cells. Miniprep DNA was prepared from the transformants and screened for the presence of the new sequence by restriction enzyme analysis. Putative correct clones were then sequenced to verify the correct sequence in the proper orientation. Plasmid Pee2 was isolated which contains the new sequence [SEQ ID NO:29] at the Bgl II site.

Step B: Construction of Pee3

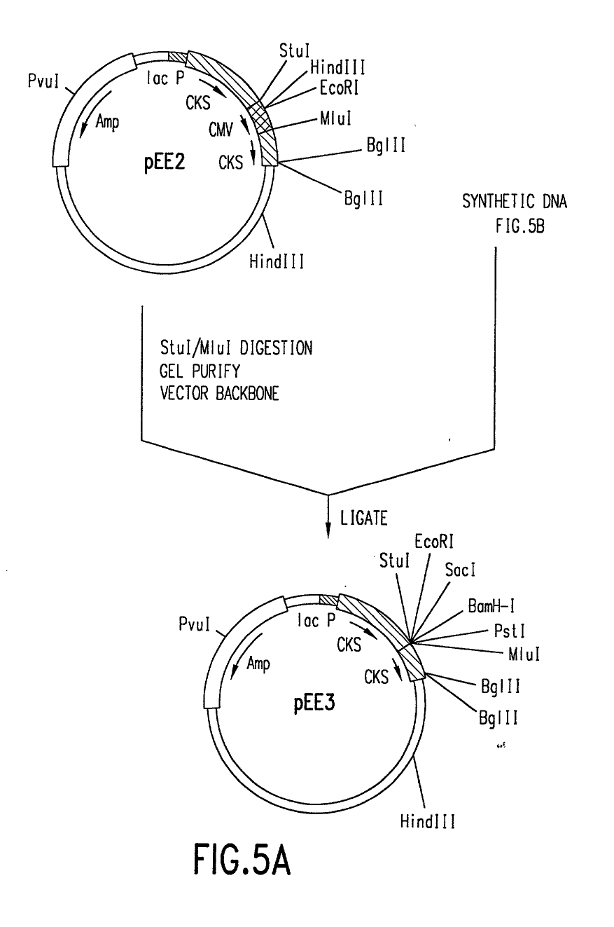

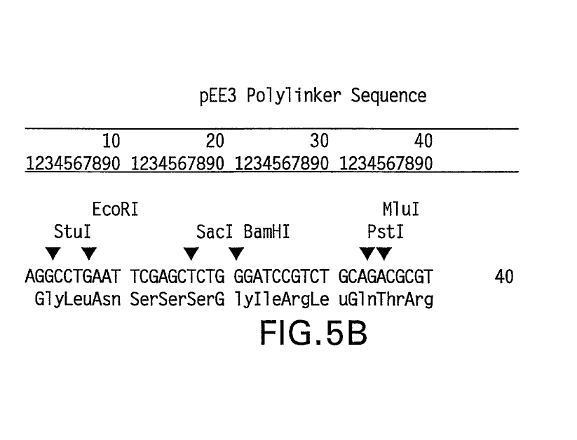

The plasmid Pee3, a derivative of the CKS expression vector Pee2 (FIG. 5A), was constructed by digesting Pee2 with StuI and MluI and cloning in a new polylinker with the following sequence (5′-3′) [SEQ ID NO:32](see FIG. 5B) and deduced amino acid sequence [SEQ ID NO:51]

AGGCCTGAATTCGAGCTCTGGGATCCGTCTGCAGACGCGT
GlyLeuAsnSerSerSerGlyIleArgLeuGlnThrArg

which contains the restriction sites StuI, EcoRI, SacI, BamH-I, PstI, and MluI.

Plasmid Pee2 was digested with StuI and MluI, and the vector backbone was purified on an agarose gel. Two oligonucleotides shown below (5′-3′) were synthesized for ligation into the Pee2/StuI/MluI backbone.

[SEQ ID NO:33]
CCTGAATTCGAGCTCTGGGATCCGTCTGCAGA
[SEQ ID NO:34]
CGCGTCTGCAGACGGATCCCAGAGCTCGAATTCAGG

These oligonucleotides were mixed together, heated to 80° C. for 10 minutes and then allowed to cool gradually overnight to 4° C. to permit annealing of the oligonucleotides. The annealed oligonucleotides were then ligated to the Pee2/StuI/MluI backbone overnight at 16° C. The ligation mixture was transformed the next day into competent XL-1 Blue cells. Miniprep DNA was prepared from the transformants and screened for the presence of the new sequence by restriction enzyme analysis. Putative correct clones were then sequenced to verify the correct sequence. Plasmid Pee3 was isolated which contains the new sequence [SEQ ID NO:32] at the StuI/MluI sites.

### EXAMPLE 11

### Construction of CKS-toxo AG-CKS Epitope-embedding Expression Vectors

The CKS expression vectors Pjo200, Peel, and Pee3 were utilized for the construction of four CKS-Toxo Ag-CKS gene fusion constructs using the Toxo P29, P30, P35, and P66 genes.

Step A: Construction of pToxo-P29: CKS-P29(1-236aa)-CKS

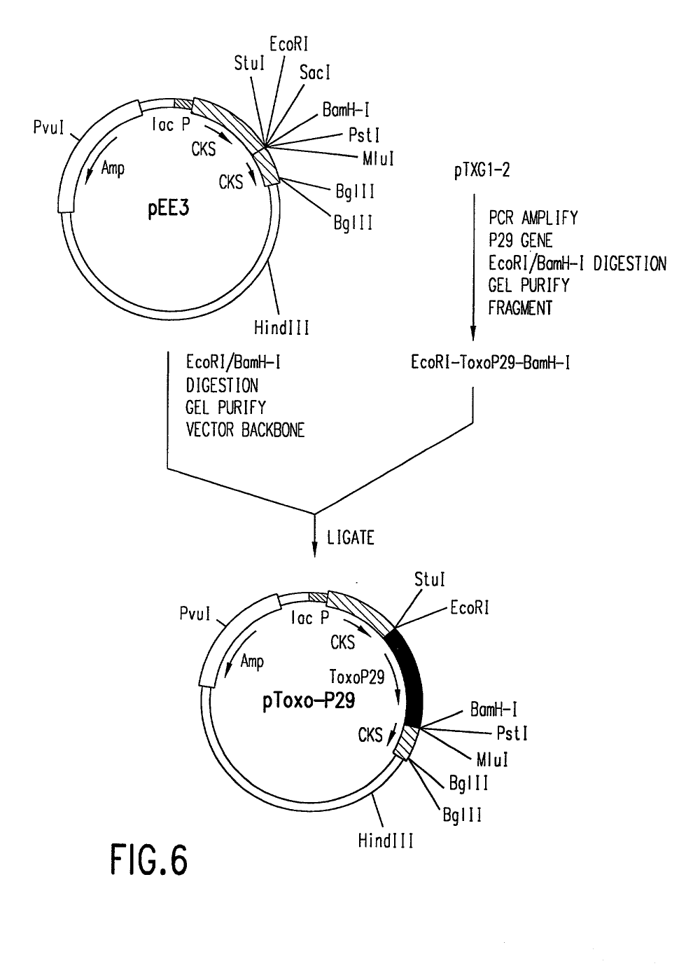

The plasmid pToxo-P29, a derivative of plasmid Pee3 (FIG. 6), was constructed by cloning a DNA fragment containing Toxo P29, obtained by PCR amplification of Toxo P29 DNA contained in plasmid Ptxg1-2 (Example 9C), into the EcoRI/BamH-I sites of Pee3. Plasmid pToxo-P29 was deposited with the ATCC under terms of the Budapest Treaty on May XX, 1998, and was accorded Accession No. ATCC XXXXX.

Large scale plasmid DNAs (Ptxg1-2 and Pee3) were isolated by general methods. Plasmid Pee3 was digested with EcoRI and BamH-I, and the vector backbone, Pee3/EcoRI/BamH-I, was purified on an agarose gel. A sense primer, starting at nucleotide 358 of the P29 gene (FIG. 3) containing an EcoRI site, and an antisense primer containing a BamH-I site, starting at nucleotide 1065 of the P29 gene, were synthesized as shown below:

Sense Primer [SEQ ID NO:35]
5′-ACTTAGAATTCGATGGCCCGACACGCAATTTTTTCC-3′
(EcoRI site is underlined)
Antisense Primer [SEQ ID NO:36]
5′-ACATGGATCCGCTGGCGGGCATCCTCCCCATCTTC-3′
(BamH-T site is underlined)

The sense and antisense primers were added to a PCR reaction mixture containing plasmid Ptxg1-2. After PCR amplification, the reaction mixture was digested with EcoRI and BamH-I, and the 708 base pair DNA fragment containing P29 was purified on an agarose gel. The purified 708 base pair DNA fragment was ligated to Pee3/EcoRI/BamH-I overnight at 16° C. The ligation mixture was transformed the next day into competent XL-1 Blue cells. Miniprep DNA was prepared from the transformants and screened for the presence of the P29 DNA sequence by restriction enzyme analysis. Plasmid pToxo-P29 contained the P29 gene embedded at the EcoRI/BamH-I sites of Pee3. This CKS-ToxoP29-CKS fusion construct was designated:

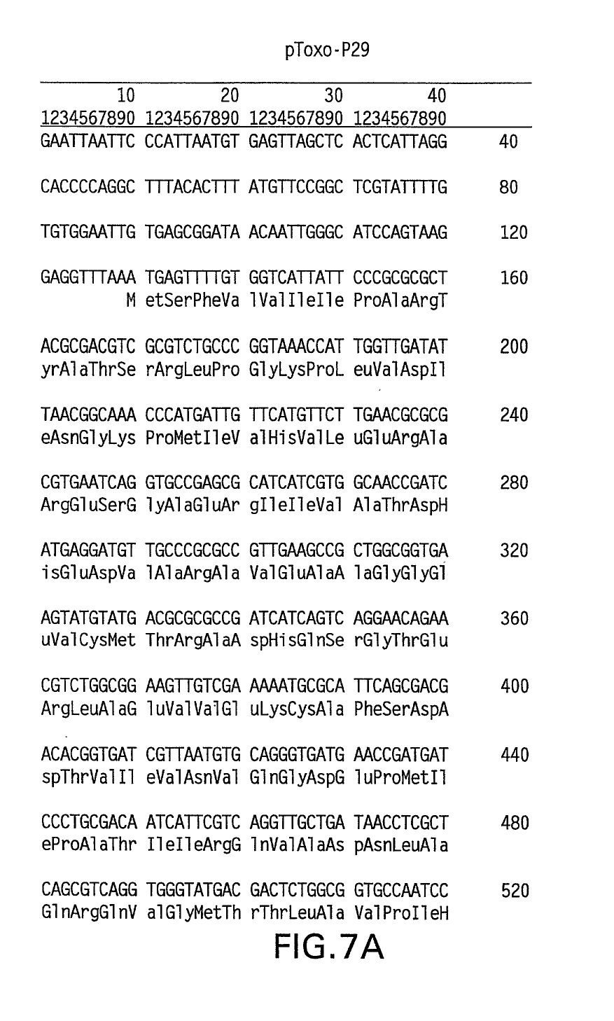

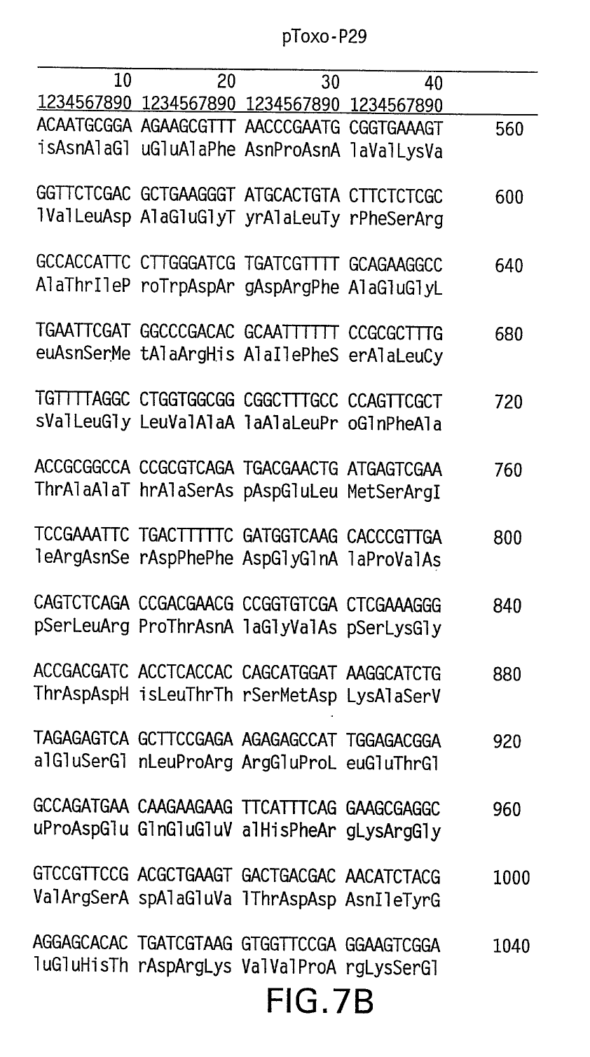

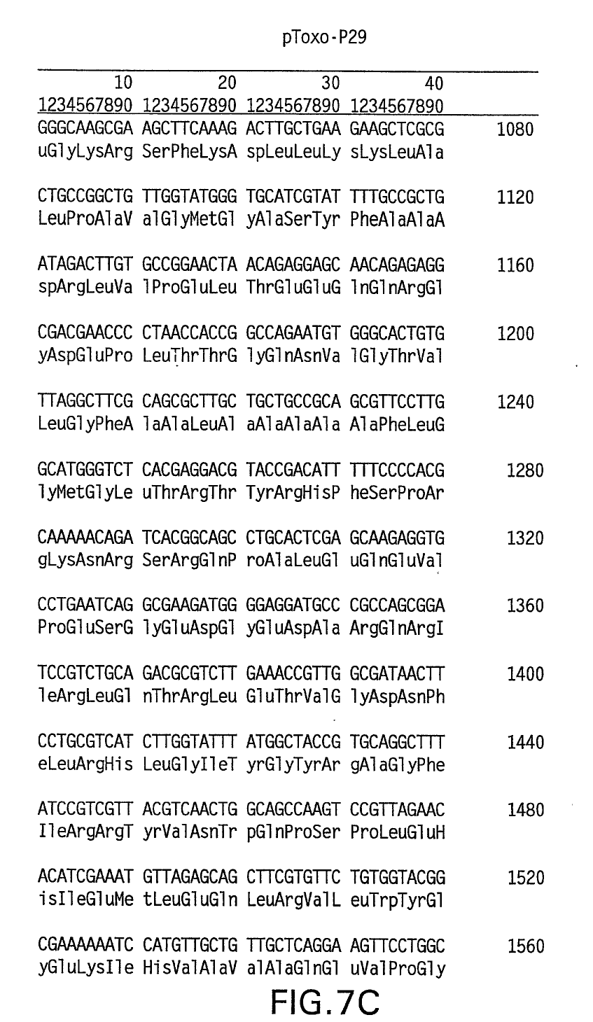

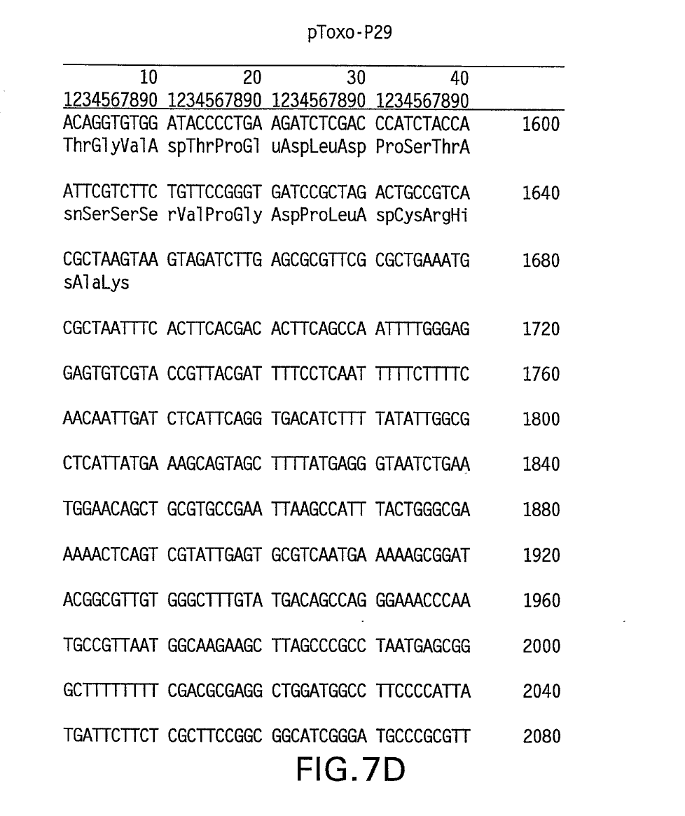

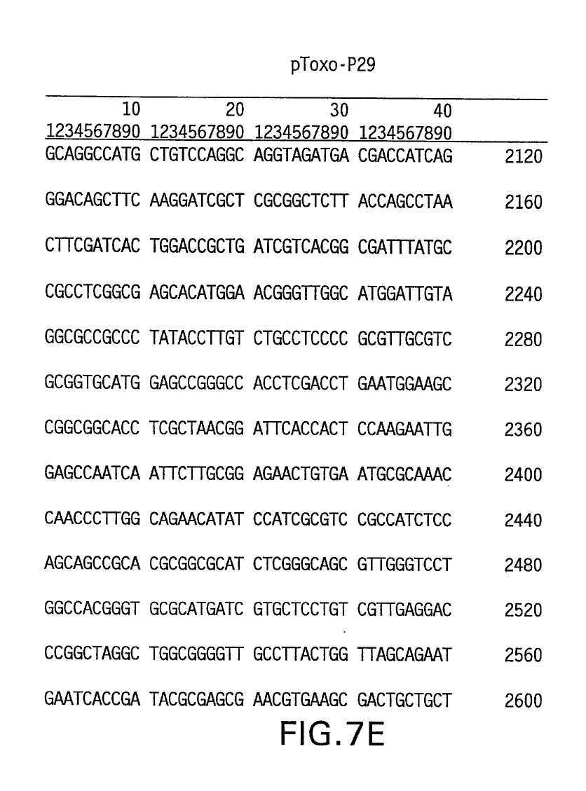

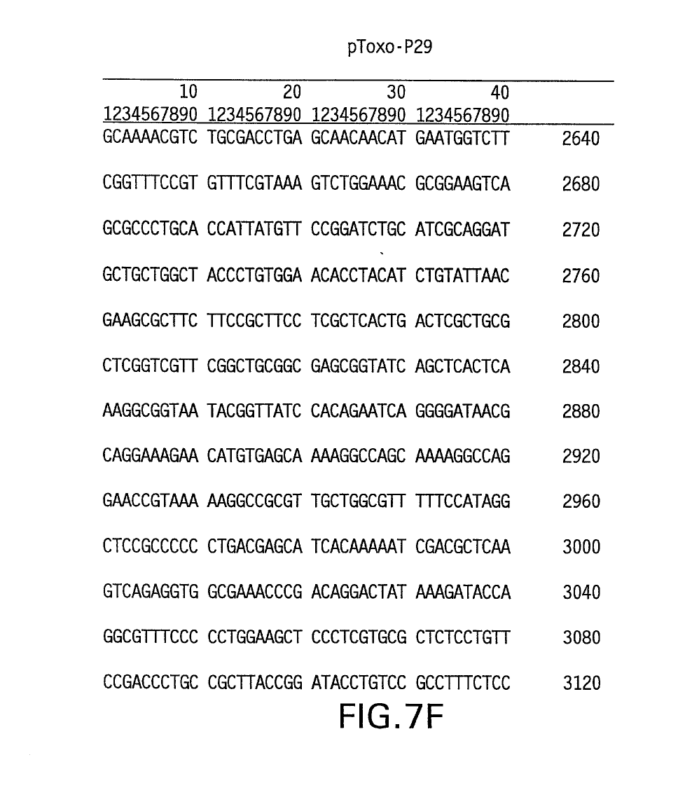

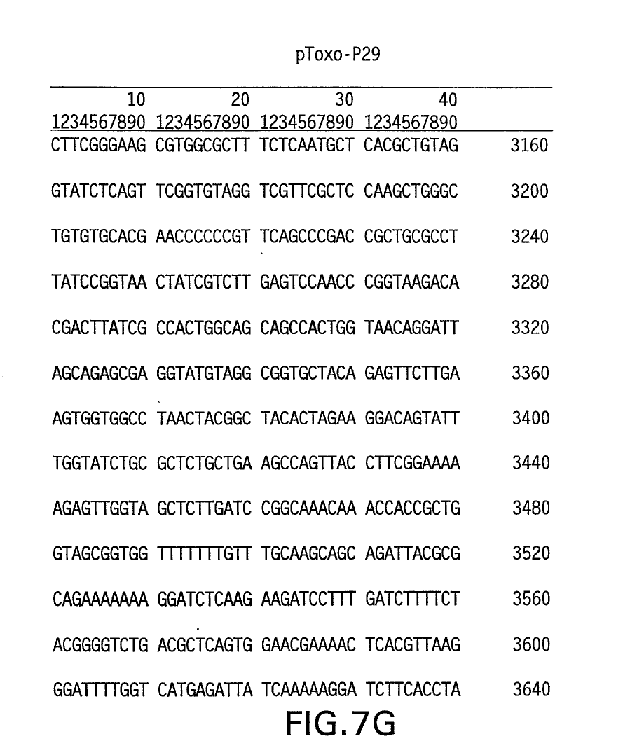

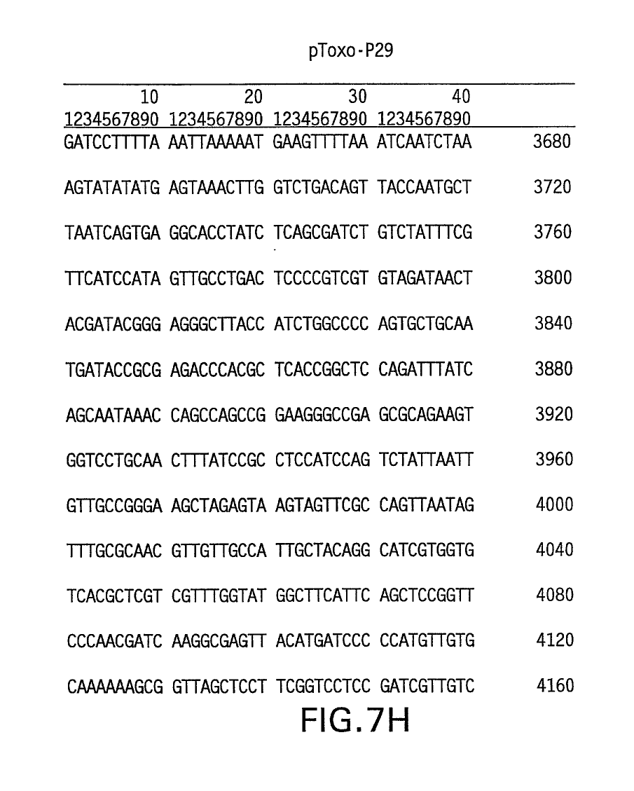

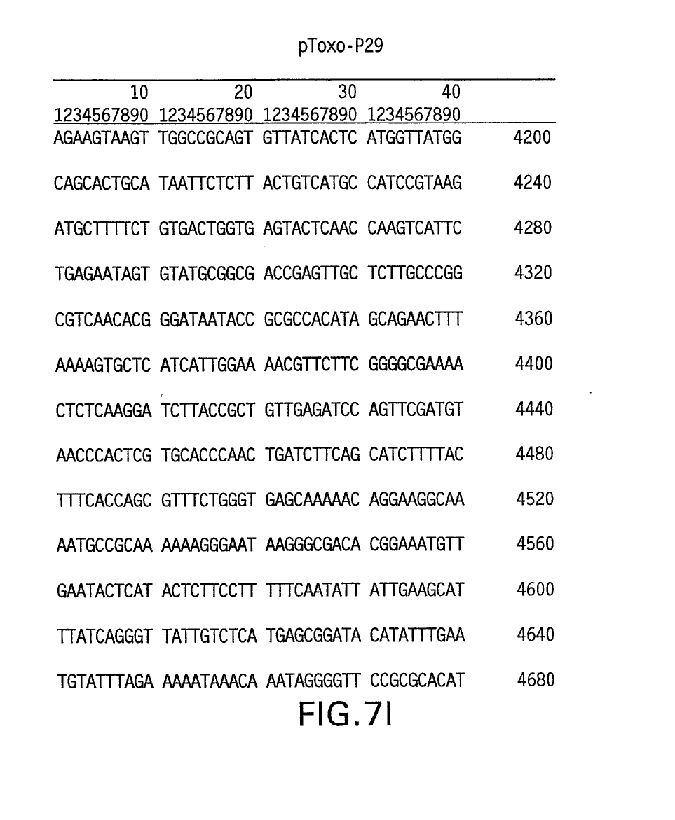

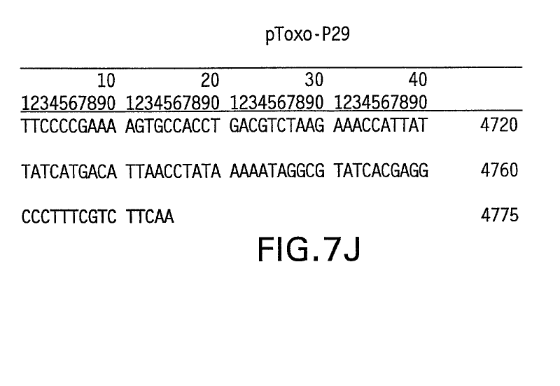

“CKS(1-171aa)-N—S-ToxoP29(1-236aa)-R-I-R-L-Q-T-R-CKS (171-260aa)”

where N, S, R, I, R, L, Q, T, R are the asparagine, serine, arginine, isoleucine, arginine, leucine, glutamine, threonine, and arginine residues, respectively, encoded by the polylinker DNA sequence of the vector. The complete DNA sequence [SEQ ID NO:37] of plasmid pToxo-P29 and the corresponding amino acid sequence [SEQ ID NO:52] of the CKS-P29-CKS fusion protein is shown are FIG. 7.

Step B: Construction of pToxo-P30:CKS-P30(1-236aa)-CKS

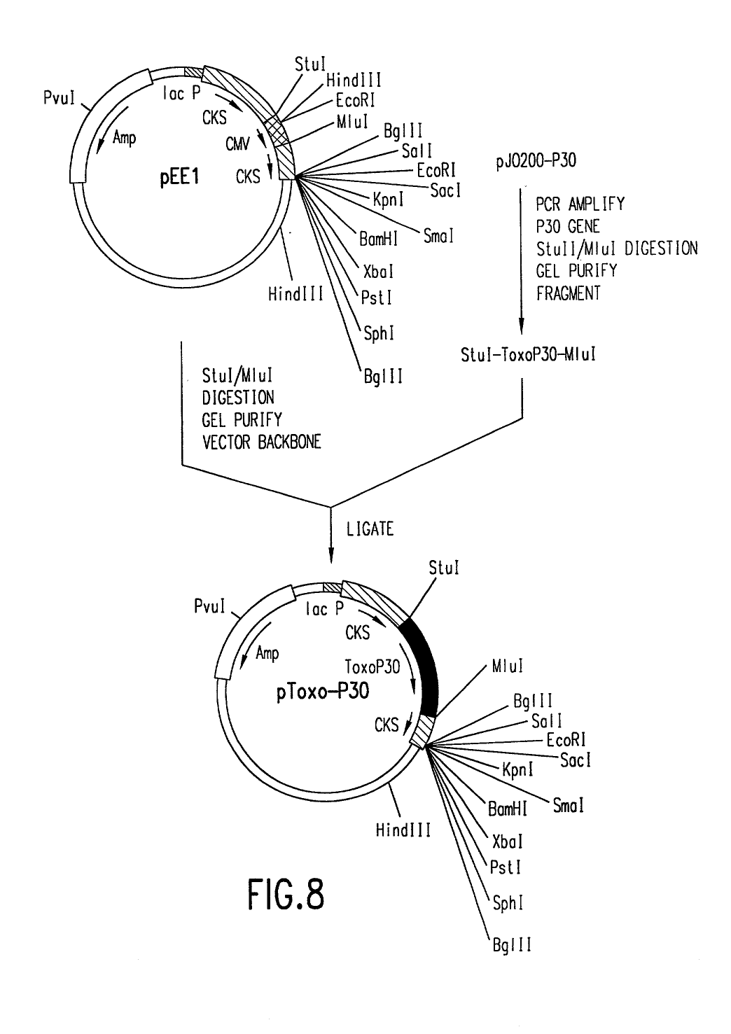

The plasmid pToxo-P30, a derivative of plasmid Peel (FIG. 8), was constructed by cloning a DNA fragment containing Toxo P30, obtained by PCR amplification of Toxo P30 DNA contained in plasmid Pjo200-P30 (Example 3A), into the StuI/MluI sites of Peel. Plasmid pToxo-P30 was deposited with the ATCC under the terms of the Budapest Treaty on May XX, 1998, and was accorded Acession No. ATCC XXXXX.

Large scale plasmid DNAs (Pjo200-P30 and Peel) were isolated by general methods. Plasmid Peel was digested with StuI and MluI, and the vector backbone, Peel/StuI/MluI, was purifed on an agarose gel. A sense primer, starting at nucleotide 464 of the P30 gene containing an StuI site, and an antisense primer containing a MluI site, starting at nucleotide 1318 of the P30 gene (Burg et al. (1988) J. Immunol. 141, 3584-3591) were synthesized as shown below:

Sense Primer [SEQ ID NO:38]
5′-TCCTAGGCCTTAATTCGATGCTTGTTGCCAATCAAG-3′
(StuI site is underlined)
Antisense Primer [SEQ ID NO:39]
5′-ACATACGCGTCGCGACACAAGCTGCGATAGAG-3′
(MluI site is underlined)

The sense and antisense primers were added to a PCR reaction mixture containing plasmid Pjo200-P30. After PCR amplification, the reaction mixture was digested with StuI and MluI, and the 855 base pair DNA fragment containing P30 was purified on an agarose gel. The purified 855 base pair DNA fragment was ligated to Peel/StuI/MluI overnight at 16° C. The ligation mixture was transformed the next day into competent XL-1 Blue cells. Miniprep DNA was prepared from the transformants and screened for the presence of the P30 DNA sequence by restriction enzyme analysis. Plasmid pToxo-P30 contained the P30 gene embedded at the StuI/MluI sites of Peel. This CKS-ToxoP30-CKS fusion construct was designated:

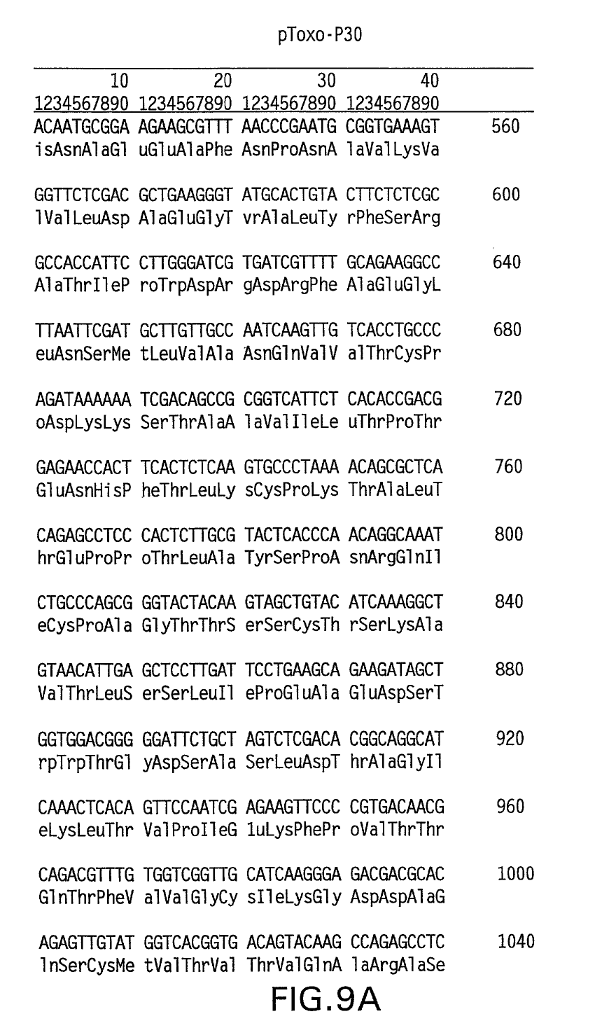

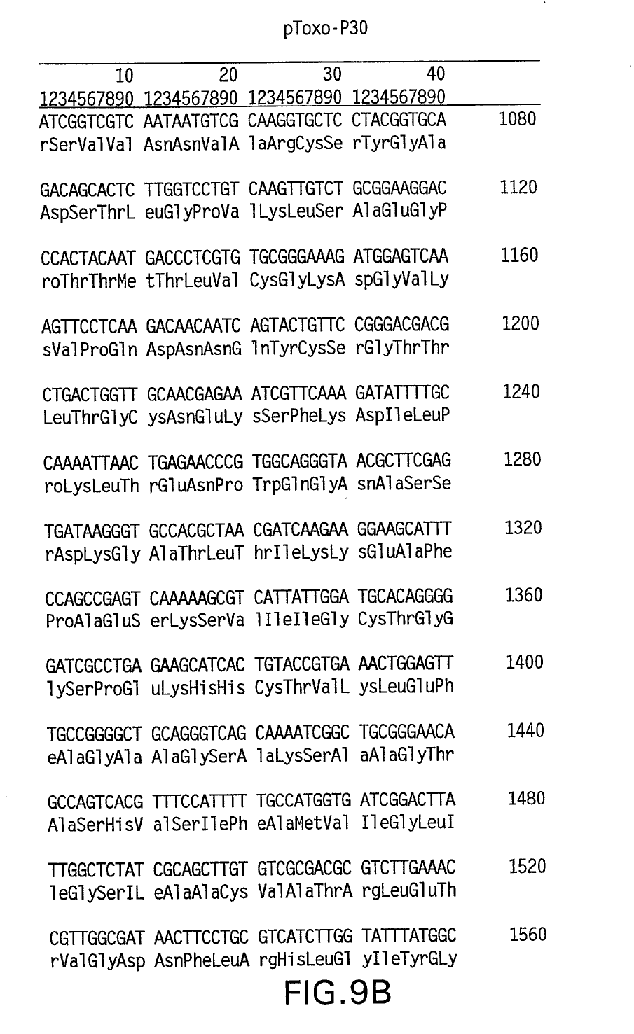

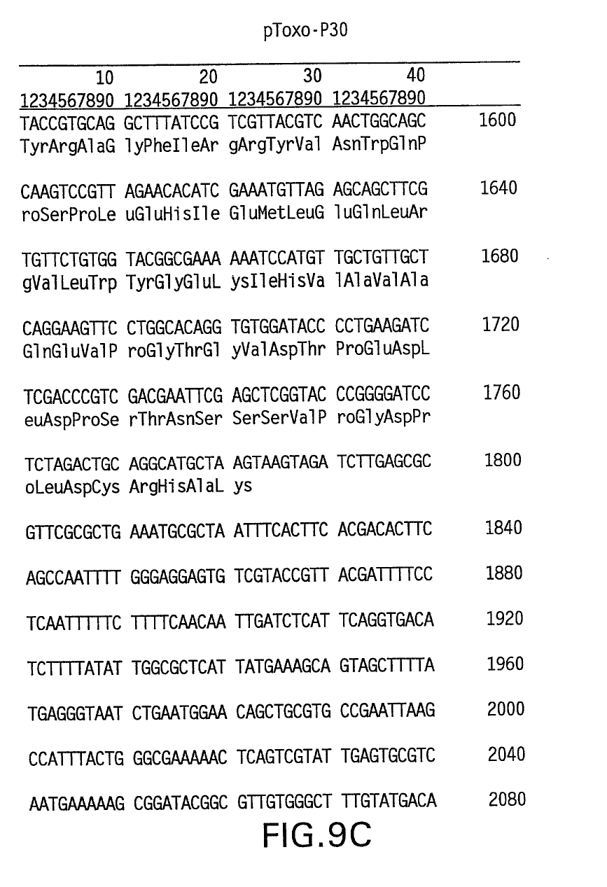

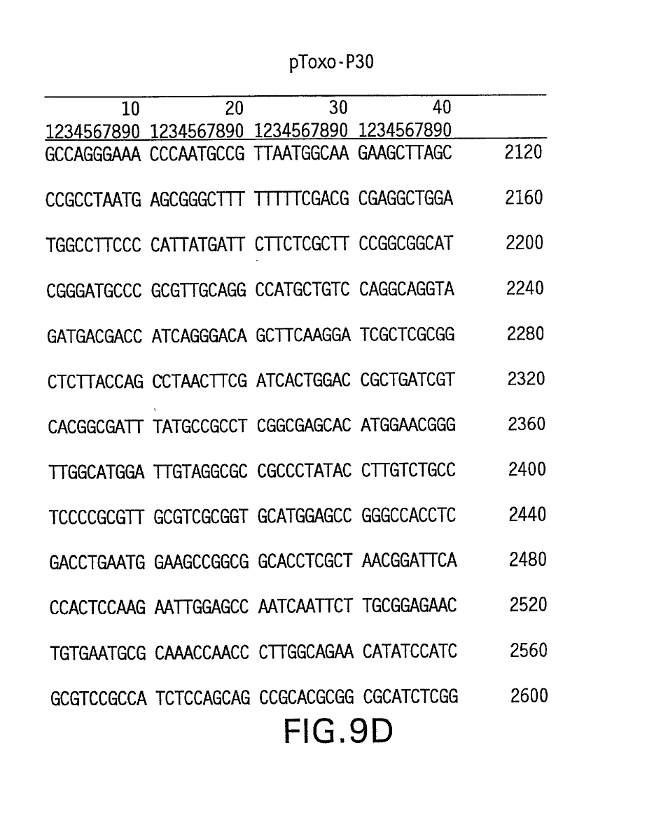

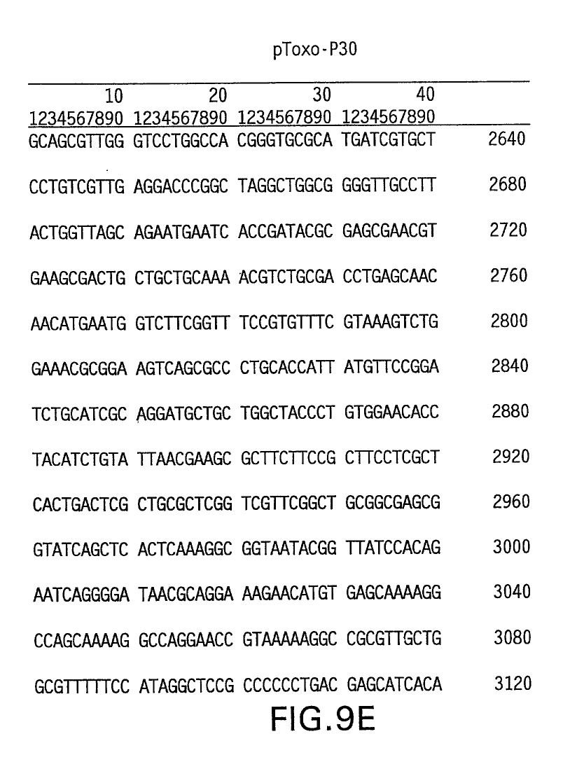

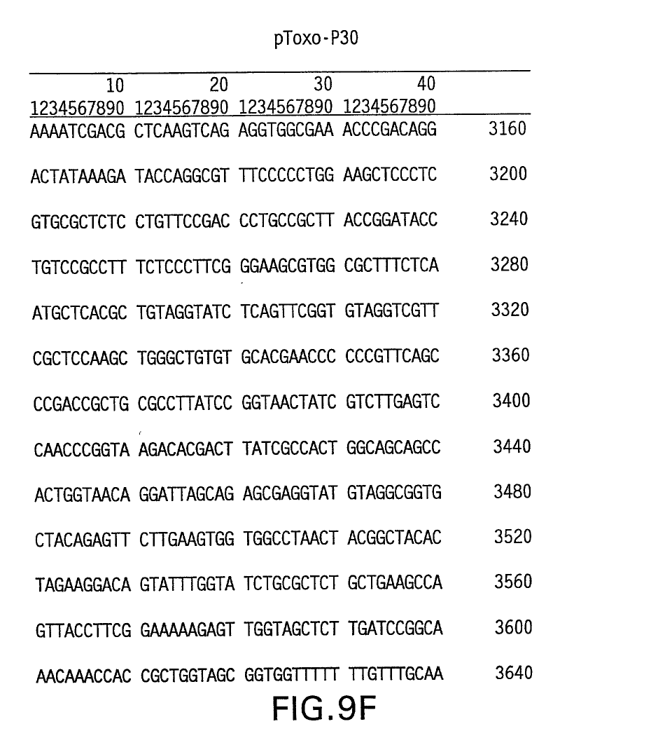

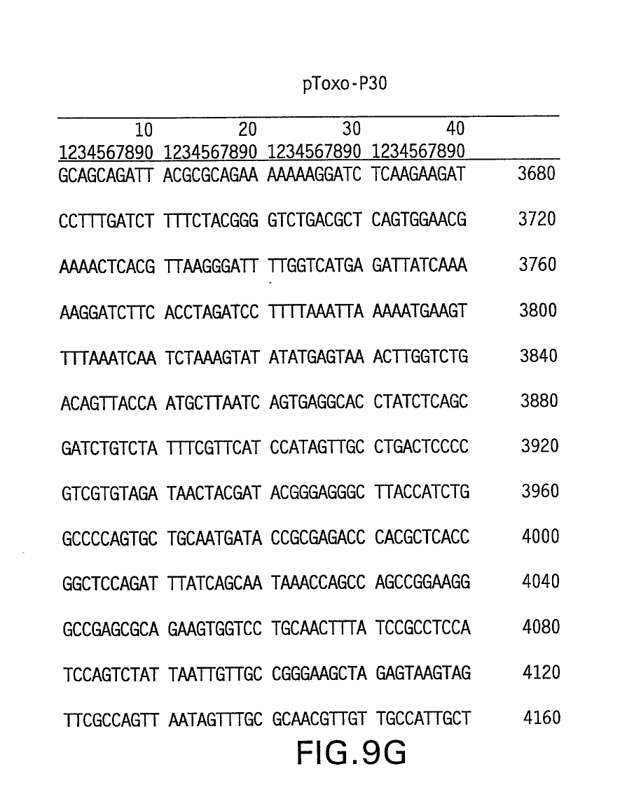

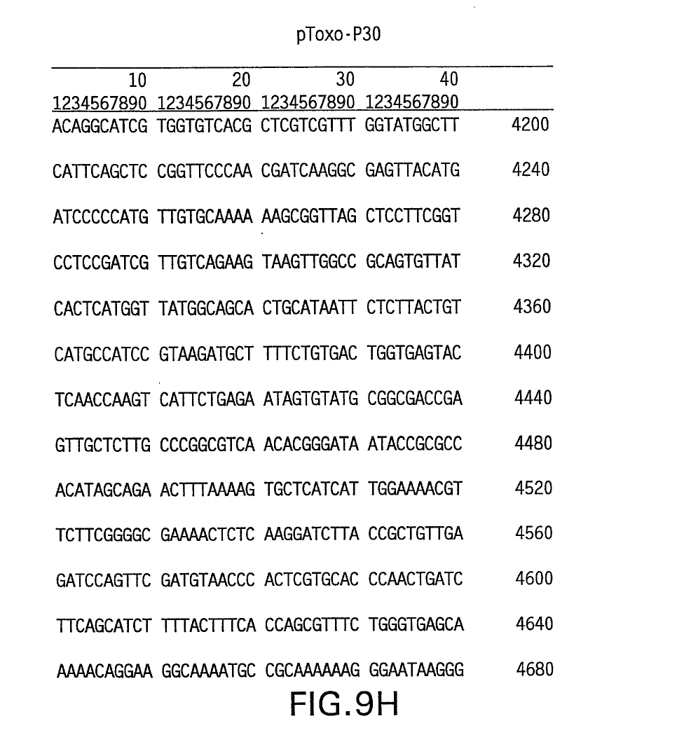

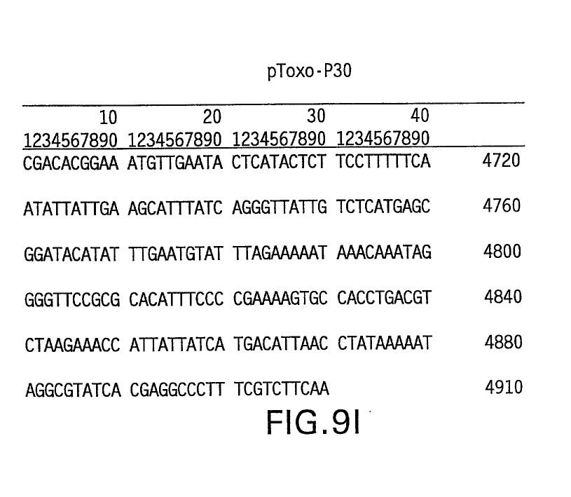

“CKS(1-171aa)-N-S-M-ToxoP30(5-289aa)-T-R-CKS(171-260aa)”

where N, S, M, T, R are the asparagine, serine, methionine, threonine, and arginine residues, respectively, encoded by the synthetic DNA sequence of the vector. The complete DNA sequence [SEQ ID NO:40] of plasmid pToxo-P30 is shown in FIG. 9 and the corresponding amino acid sequence [SEQ ID NO:53] of the CKS-P30-CKS fusion protein are shown in FIG. 9.

Step C: Construction of pToxo-P35S:CKS-P35(1-135aa)-CKS

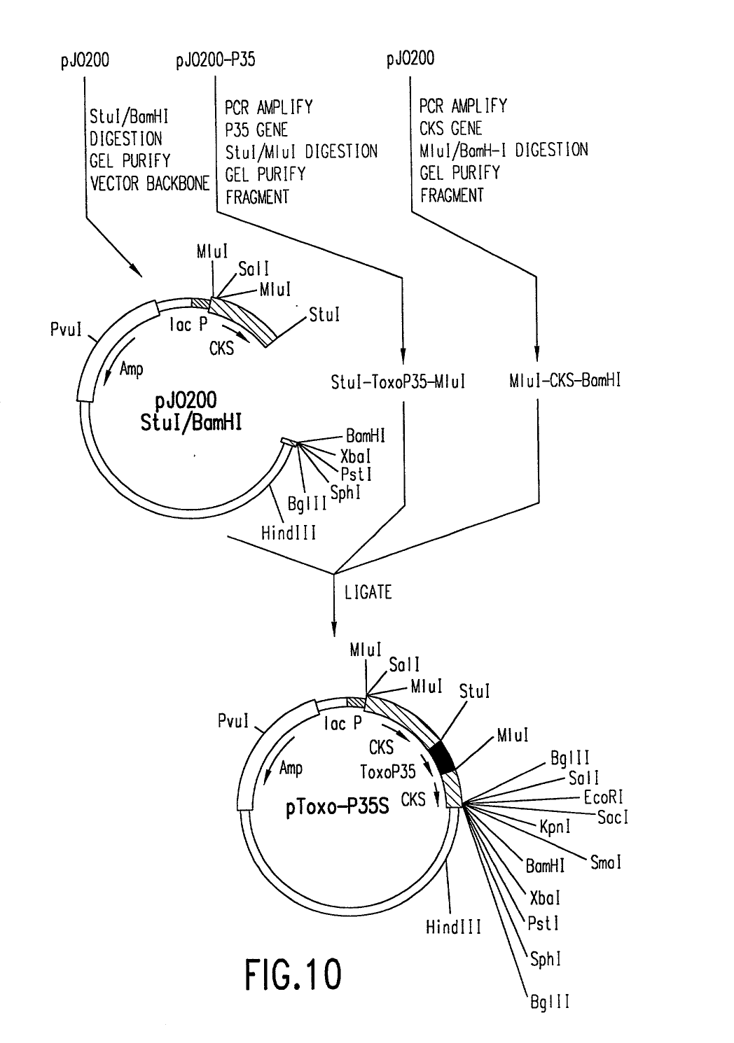

The plasmid pToxo-P35S, a derivative of plasmid Pjo200 (FIG. 10), was constructed by cloning a DNA fragment containing Toxo P35, obtained by PCR amplification of Toxo P35 DNA contained in plasmid Pjo200-P35 (Example 3A), into the StuI site of Pjo200. Plasmid pToxo-P35S was deposited with the ATCC under terms of the Budapest Treaty on May XX, 1998, and was accorded Accession No. ATCC XXXXX.

Large scale plasmid DNAs (Pjo200-P35 and Pjo200) were isolated by general methods. Plasmid Pjo200 was digested with StuI and BamH-I, and the vector backbone, Pjo200/StuI/BamH-I, was purified on an agarose gel. A sense primer, starting at nucleotide 91 of the P35 gene containing an StuI site, and an antisense primer containing a MluI site, starting at nucleotide 495 of the P35 gene (Knapp et al., 1989 (EPA 431541A2)) were synthesized as shown below:

Sense Primer [SEQ ID NO:41]
5′-GAGCAGAAGGCCTTATGAACGGTCCTTTGAGTTATCATCC-3′
(StuI site is underlined)
Antisense Primer [SEQ ID NO:42]
5′-TTCGCTCACGCGTATGGTGAACTGCCGGTATCT-3′
(MluT site is underlined)

The sense and antisense primers were added to a PCR reaction mixture containing plasmid Pjo200-P35. After PCR amplification, the reaction mixture was digested with StuI and MluI, and the 405 base pair DNA fragment containing P35 was purified on an agarose gel. A sense primer, starting at nucleotide 640 of Pjo200 containing an MluI site, and an antisense primer starting at nucleotide 905 of Pjo200 were synthesized as shown below:

Sense Primer [SEQ ID NO:43]
5′-GACGGAGACGCGTCTTGAACCGTTGGCGATAACT-3′
(MluI site is underlined)
Antisense Primer [SEQ ID NO:44]
5′-GCATGCCTGCAGTCTAGAGGA-3′

The sense and antisense primers were added to a PCR reaction mixture containing plasmid Pjo200. After PCR amplification, the reaction mixture was digested with MluI and BamH-I, and the 266 base pair DNA fragment containing P35 was purified on an agarose gel.

The purified 405 base pair DNA fragment containing the P35 gene and the purified 266 base pair DNA fragment containing the 3′ end of the CKS gene, were ligated to Pjo200/StuI/BamH-I overnight at 16° C. The ligation mixture was transformed the next day into competent XL-1 Blue cells. Miniprep DNA was prepared from the transformants and screened for the presence of the P35 DNA sequence by restriction enzyme analysis. Plasmid pToxo-P35S contained the P35 gene embedded at the StuI/MluI sites of Pjo200. This CKS-ToxoP35-CKS fusion construct was designated:

“CKS(1-171aa)-ToxoP35(1-135aa)-T-R-CKS(171-260aa)”

where T and R are the threonine and arginine residues, respectively, encoded by the synthetic DNA sequence of the vector. The complete DNA sequence [SEQ ID NO:45] of plasmid pToxo-P35S and the corresponding amino acid sequence [SEQ ID NO:54] of the CKS-P35-CKS fusion protein are shown in FIG. 11.

Step D: Construction of pToxo-P66g:

CKS-P66(26-428aa)-CKS

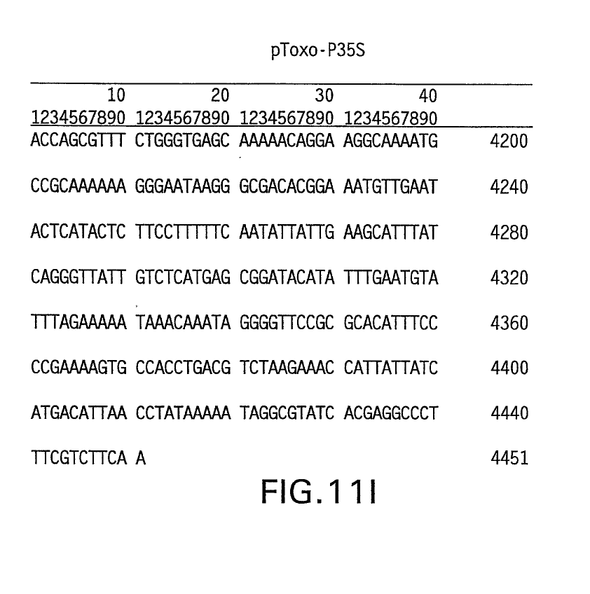

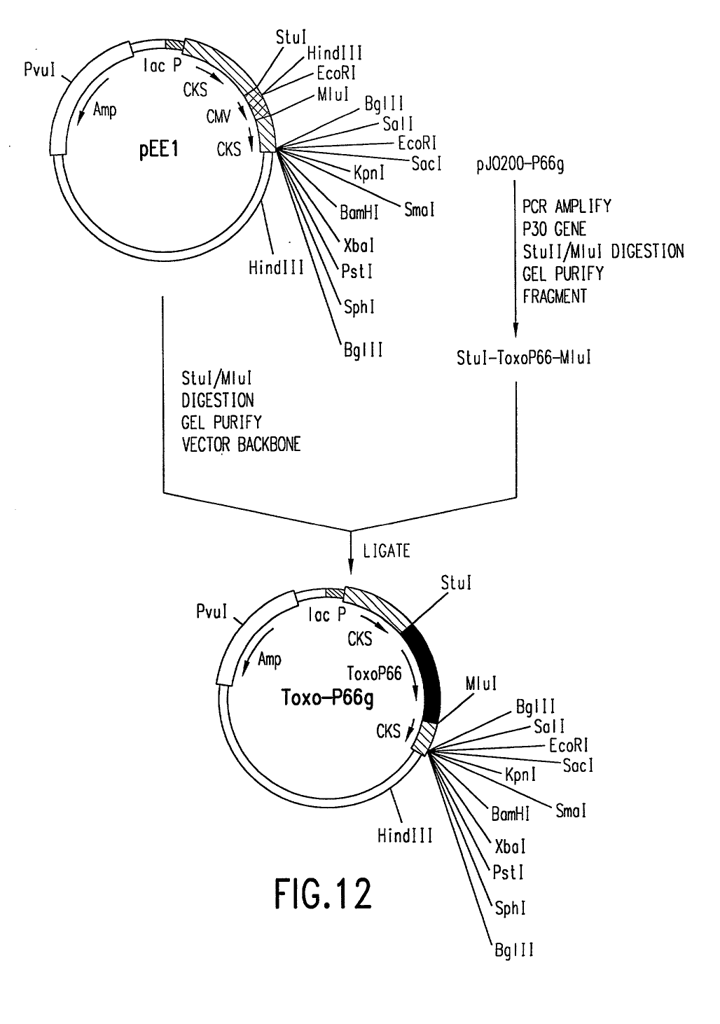

The plasmid pToxo-66g, a derivative of plasmid Peel (FIG. 12), was constructed by cloning a DNA fragment containing Toxo P66, obtained by PCR amplification of Toxo P66 DNA contained in plasmid Pjo200-P66g (Example 3A), into the StuI/MluI sites of Peel. Plasmid pToxo-P66g was deposited with the ATCC under terms of the Budapest Treaty on May XX, 1998, and was accorded Accession No. ATCC XXXXX.

Large scale plasmid DNAs (Pjo200-P66g and Peel) were isolated by general methods. Plasmid Peel was digested with StuI and MluI, and the vector backbone, Peel/StuI/MluI, was purified on an agarose gel. A sense primer, starting at nucleotide 122 of the P30 gene containing an StuI site, and an antisense primer containing a MluI site, starting at nucleotide 1330 of the P66 gene (Knapp et al., supra (1989)) were synthesized as shown below:

Sense Primer [SEQ ID NO:46]
5′-ATATTAGGCCTTATGAGCCACAATGGAGTCCCCGCTTATCC-3′
(StuI site is underlined)
Antisense Primer [SEQ ID NO:47]
5′-CAGTGTACGCGTTTGCGATCCATCATCCTGcTCTCTTC-3′
(MluT site is underlined)

The sense and antisense primers were added to a PCR reaction mixture containing plasmid Pjo200-P66g. After PCR amplification, the reaction mixture was digested with StuI and MluI, and the 1209 base pair DNA fragment containing P66 was purified on an agarose gel. The purified 1209 base pair DNA fragment was ligated to Peel/StuI/MluI overnight at 16° C. The ligation mixture was transformed the next day into competent XL-1 Blue cells. Miniprep DNA was prepared from the transformants and screened for the presence of the P66 DNA sequence by restriction enzyme analysis. Plasmid pToxo-P66g contained the P66 gene embedded at the StuI/MluI sites of Peel. This CKS-ToxoP66-CKS fusion construct was designated:

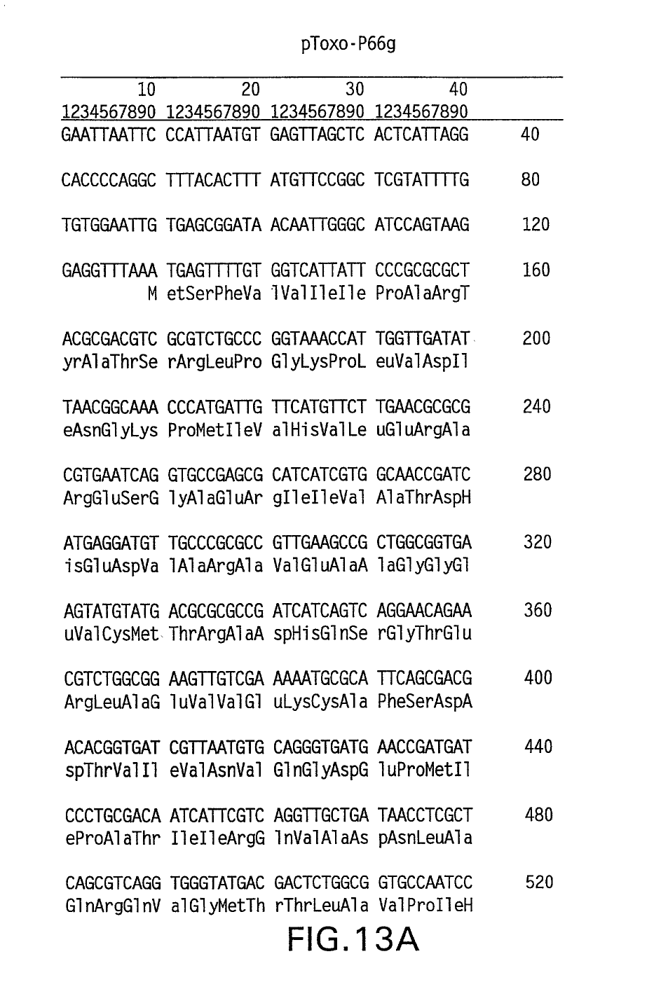

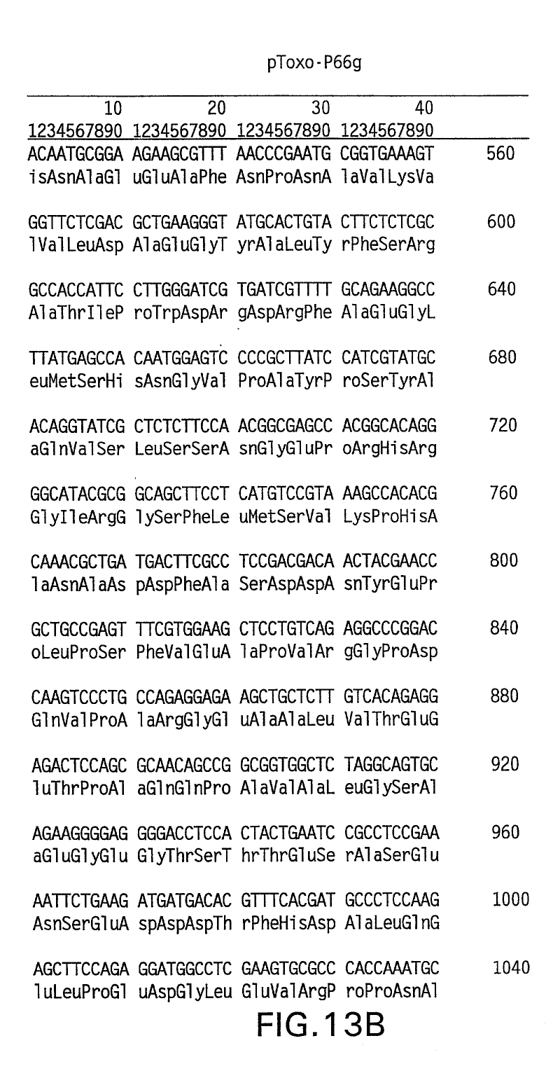

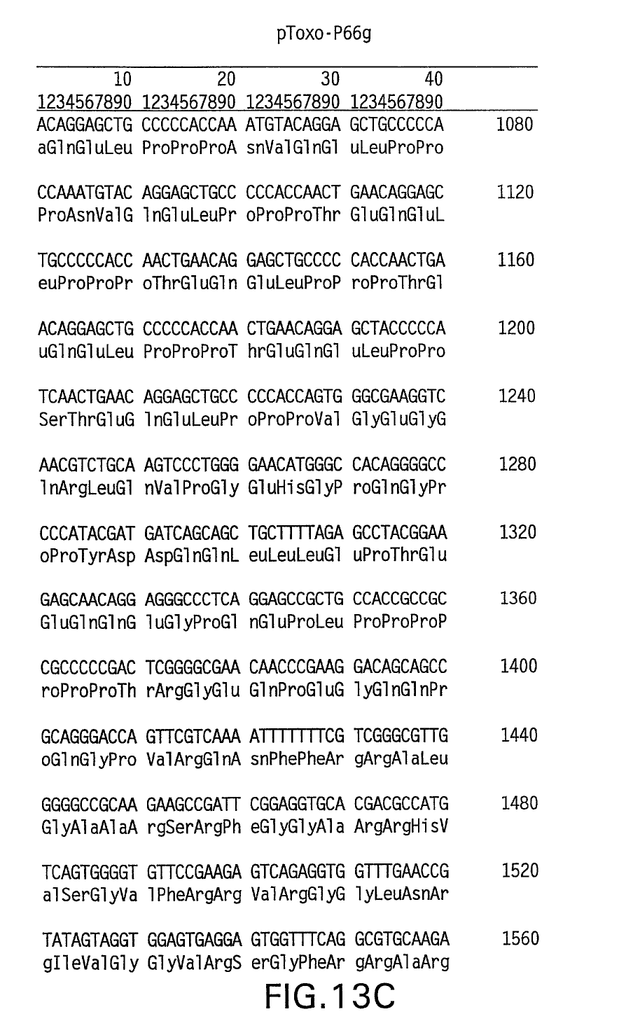

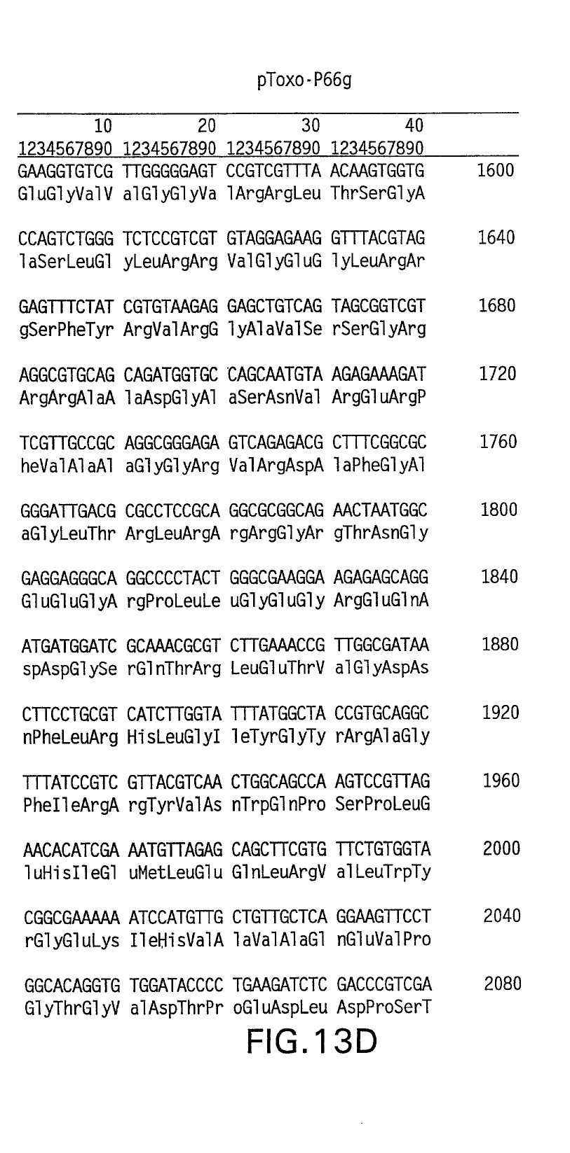

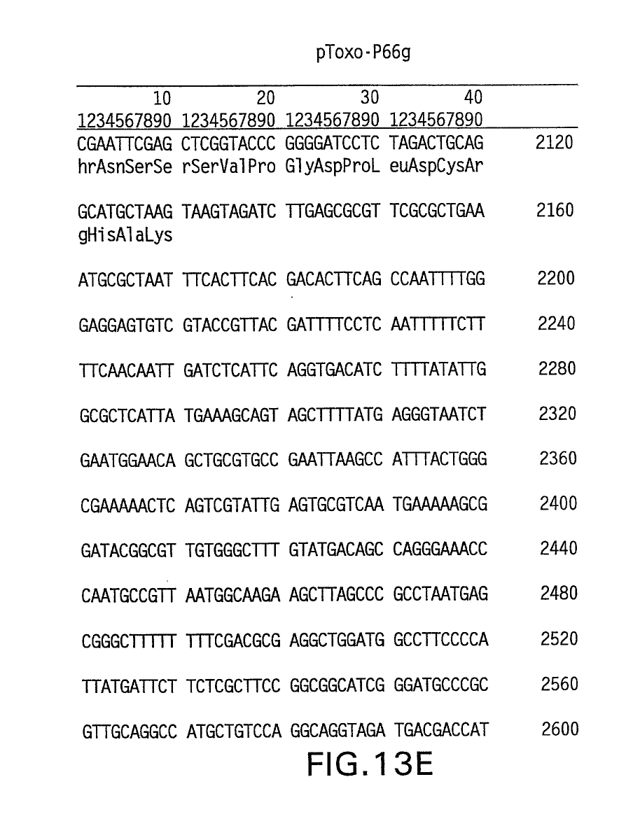

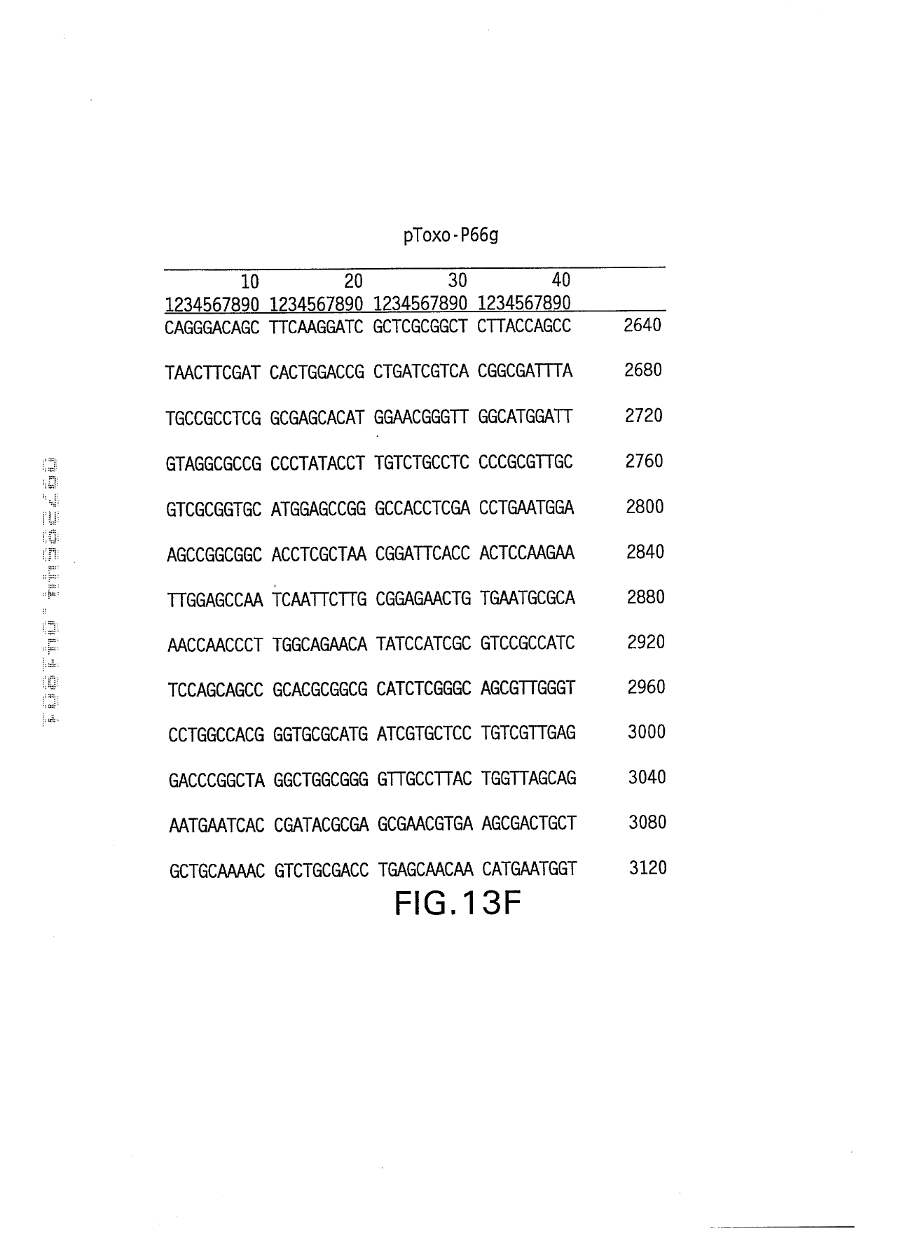

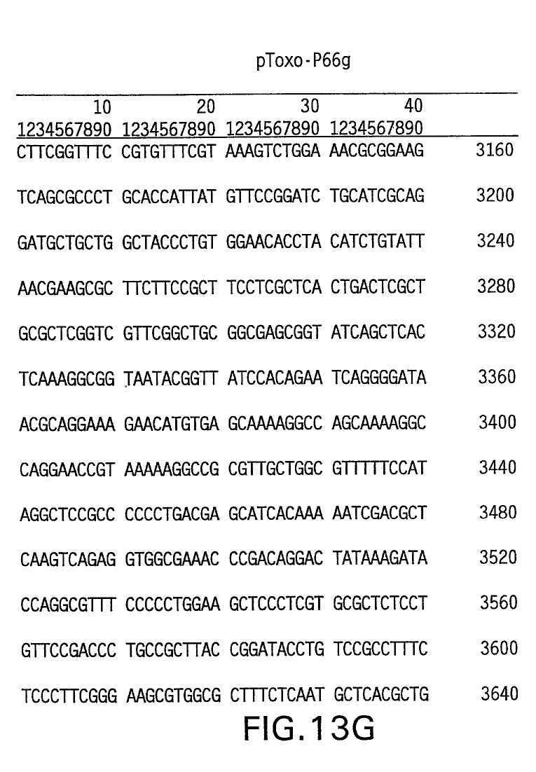

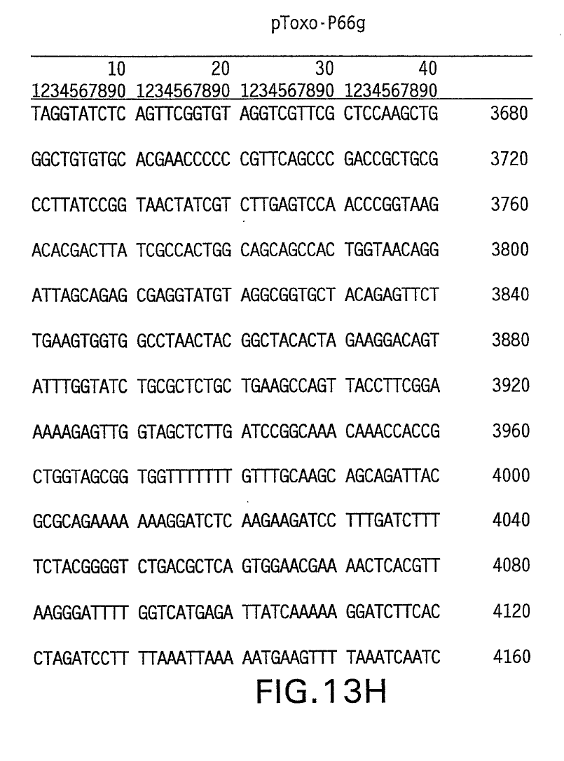

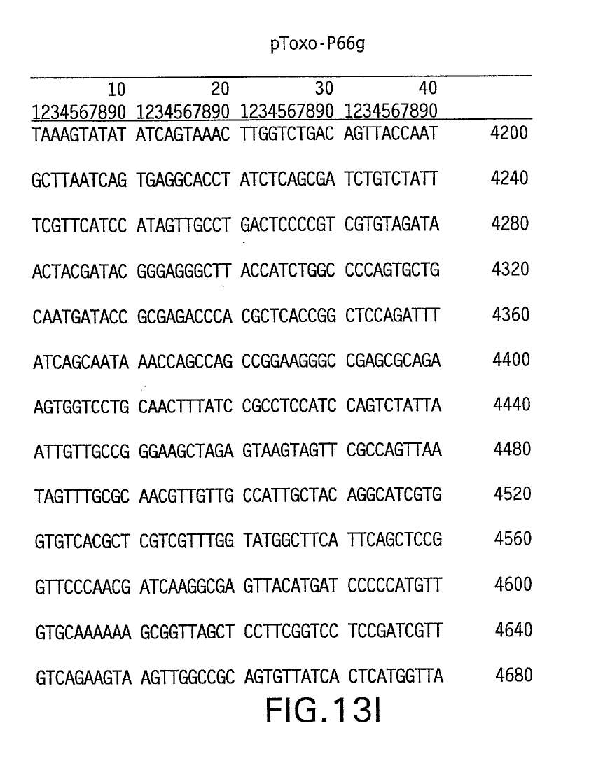

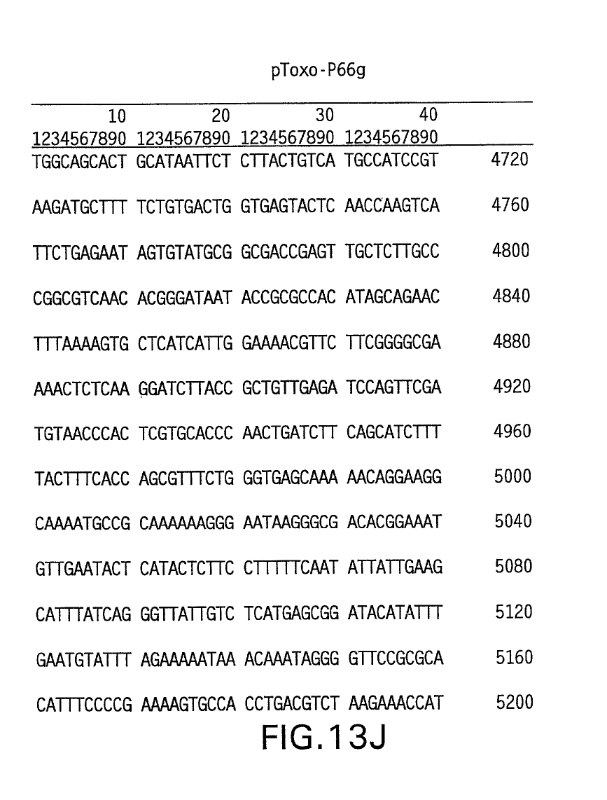

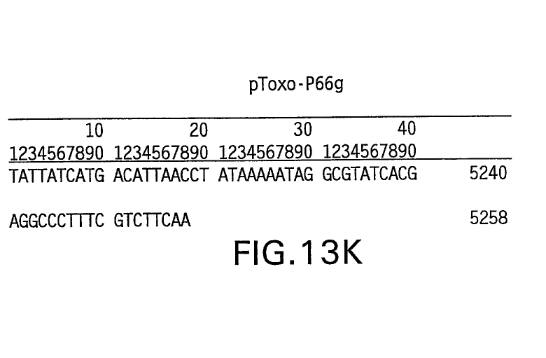

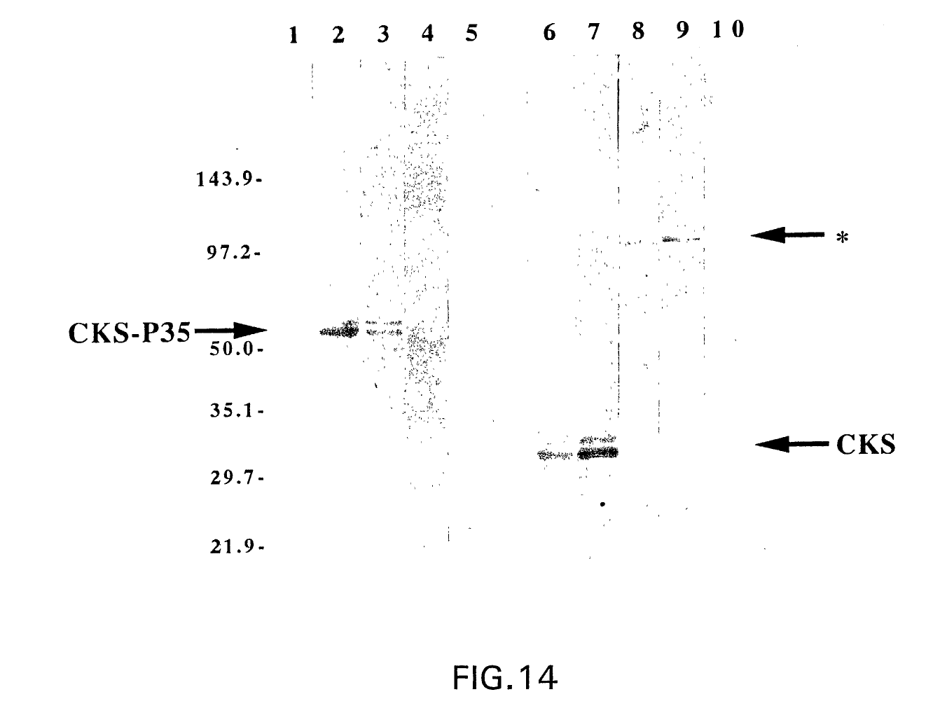

“CKS(1-171aa)-M-ToxoP66(26-428aa)-T-R-CKS(171-260aa)”

where M, T, and R are the methionine, threonine and arginine residues, respectively, encoded by the synthetic DNA sequence of the vector. The complete DNA sequence [SEQ ID NO:48] of plasmid pToxo-P66g and the corresponding amino acid sequence [SEQ ID NO:55] of the CKS-P66-CKS are shown in FIG. 13.

### EXAMPLE 12

### Development of a Toxo Recombinant Antigen Cocktail for the Detection of Toxoplasma-specific IgG and IgM

The results in Tables 1 and 2 of Example 6B indicated that more than one recombinant antigen would be required to detect Toxoplasma-specific IgG and IgM in order to replace the tachyzoite in an immunoassay. Additional sera were sourced from patients with an acute or chronic Toxolasmosis and tested with the individual antigens coated in separate wells listed in Tables 1 and 2 using the IgG or IgM Microtiter ELISA described in Example 6B. These results indicated that a cocktail of recombinant antigens necessary and sufficient to replace the tachyzoite in an immunoassay should be composed of the following Toxo antigens:

Toxo IgG Immunoassay: P29+P30+P35

Toxo IgM Immunoassay: P29+P35+P66

In order to demonstrate the diagnostic utility of the Toxo recombinant antigens in the proposed above combinations in an immunoassay, i.e. the coating of the Toxo antigens P29, P30, and P35 in a single microtiter plate well (Microtiter format) or other solid phase, e.g. microparticles (MEIA format), to detect Toxoplasma-specific IgG antibodies and the coating of the Toxo antigens P29, P35, and P66 in a single microtiter plate well (Microtiter format) or other solid phase, e.g. microparticles (MEIA format), to detect Toxoplasma-specific IgM antibodies, the following experiments were performed:

Step A: Expression of cloned genes in E. coli

Bacterial clones pToxo-P29, pToxo-P30, pToxo-P35S, and pToxo-P66g expressing the CKS fusion proteins rpToxo-P29, rpToxo-P30, rpToxo-P35S, and rpToxo-P66g, respectively, were grown in SUPERBROTH II media containing 100 ug/ml ampicillin to log phase, and the synthesis of the CKS-Toxo fusion protein was induced by the addition of IPTG as previously described (Robinson et al. (1993) J. Clin. Micro. 31, 629-635). After 4 hours post-induction, the cells were harvested, and the cell pellets were stored at −80° C. until protein purification.

Step B: Purification of Recombinant Toxo Antigens

Insoluble recombinant antigens rpToxo-P29, rpToxo-P30, rpToxo-P35S, and rpToxo-P66g were purified after lysis from cell paste by a combination of detergent washes followed by solubilization in 8M urea (Robinson et al., supra (1993)). After solubilization was complete, these proteins were filtered through a 0.2 m filter and either stored at 2-8° C. (w/urea) or dialyzed against 50 Mm Tris, Ph 8.5 and then stored at 2-8° C. (w/o urea)

Step C: Human Sera for Testing

Four groups of serum specimens from a French population were evaluated for the presence of Toxoplasma-specific IgG and IgM antibodies using the Microtiter ELISA. These serum specimens collectively cover the entire span of Toxoplasma infection from early seroconversion (acute toxoplasmosis) to convalesence (latent infection, chronic toxoplasmosis) and represent the types of specimens normally encountered in routine Toxoplasma serology.

Group 1: Negative Serum Specimens

This group contained 200 serum specimens negative for Toxoplasma IgG and IgM antibodies as determined by the Abbott Imx Toxo IgG and IgM immunoassays.

Group 2: “Ancienne” Serum Specimens

This group contained 100 serum specimens negative for Toxoplasma IgM antibodies and positive for Toxoplasma IgG antibodies by the Abbott Imx Toxo IgG and IgM immunoassays. These specimens were negative for Toxoplasma IgA antibodies as determined by an immunocapture assay using a suspension of tachyzoites (IC-A) (Pinon, J. M. (1986) Diag. Immunol. 4:223-227).

Group 3: “Évolutive” Serum Specimens

This group contained 99 serum specimens positive for Toxoplasma IgG antibodies by a high sensitivity direct agglutination assay (HSDA) (Desmonts, G. and Remington, J. S. (1980) J. Clin. Micro. 11:562-568) and positive for Toxoplasma IgM and IgA antibodies using a specific immunocapture assay (IC-M, IC-A).

Group 4: “Précoce” Serum Specimens

This group contained 66 specimens sourced from individuals with evidence of a early seroconversion of Toxoplasma-specific antibodies (absence or early manifestation of IgG antibodies and positive for IgM and IgA antibodies using a specific immunocapture assay (IC-M, IC-A)).

Step D: Evaluation of Human Sera in the Recombinant Toxo Antigen Microtiter ELISA

Purified recombinant Toxo antigens (Example 12B) were coated onto the wells of the microtiter plate as follows:

For the IgG microtiter ELISA, the three Toxo antigens rpToxo-P29, rpToxo-P30, and rpToxo-P35S (w or w/o urea) were diluted together into PBS to a final concentration of 5 ug/ml for each antigen, and plates were coated and processed as described in Example 6B using a goat anti-human IgG-HRPO conjugate to detect bound human IgG. All three Toxo antigens were coated together into the same microtiter well to detect Toxoplasma-specific IgG. For the IgM microtiter ELISA, the three Toxo antigens rpToxo-P29, rpToxo-P35S, and rpToxo-P66g (w or w/o urea) were diluted together into PBS to a final concentration of 5 mg/ml for each antigen, and plates were coated and processed as described in Example 6B using a goat anti-human IgM-HRPO conjugate to detect bound human IgM. All three Toxo antigens were coated together into the same microtiter well to detect Toxoplasma-specific IgM. The cut-off for these assays was between 2 to 3 standard deviations from the mean of the negative population.

Step E: Results of the Evaluation of Human Sera in the Recombinant Toxo Antigen (P29+P30+P35) IgG Microtiter ELISA

The serum specimens from Groups 1-4 (Example 12C) were tested for the presence of Toxoplasma-specific IgG using the recombinant Toxo antigen IgG microtiter ELISA (rpToxo-P29 (P29)+rpToxo-P30 (P30)+rpToxo-P35S (P35)). The results from this evaluation are presented in Tables 4-8.

TABLE 4
Evaluation of Group 1 Negative Serum Specimens by
Toxo IgG Microtiter ELISA

Abbott Imx Toxo IgG

| | | Pos | Neg |
|---|---|---|---|
| Toxo IgG | Pos | 0 | 8 |
| (P29 + P30 + P35) | Neg | 0 | 192 |
| Microtiter | | | |
| ELISA | | | |

Specificity: 192/200 = 96%

TABLE 5
Evaluation of Group 2 “Ancienne” Serum Specimens by
Toxo IgG Microtiter ELISA

Abbott Imx Toxo IgG

| | | Pos | Neg |
|---|---|---|---|
| Toxo IgG | Pos | 97 | 0 |
| (P29 + P30 + P35) | Neg | 3 | 0 |
| Microtiter | | | |
| ELISA | | | |

Sensitivity: 97/100 = 97%

TABLE 6
Evaluation of Group 3 “Évolutive” Serum
Specimens by Toxo IgG Microtiter ELISA

HSDA IgG

| | | Pos | Neg |
|---|---|---|---|
| Toxo IgG | Pos | 99 | 0 |
| (P29 + P30 + P35) | Neg | 0 | 0 |
| Microtiter | | | |
| ELISA | | | |

Sensitivity: 99/99 = 100%

TABLE 7
Evaluation of Group 4 “Précoce” Serum Specimens by
Toxo IgG Microtiter ELISA

HSDA IgG

| | | Pos | Neg |
|---|---|---|---|
| Toxo IgG | Pos | 54 | 1 |
| (P29 + P30 + P35) | Neg | 1 | 10 |
| Microtiter | | | |
| ELISA | | | |

Sensitivity: 54/55 = 98.1%

TABLE 8
Summary of Evaluation of Groups 1-4 Serum Specimens
by Toxo IgG Microtiter ELISA

Reference Test

| | | Pos | Neg |
|---|---|---|---|
| Toxo IgG | Pos | 250 | 9 |
| (P29 + P30 + P35) | Neg | 4 | 202 |
| Microtiter | | | |
| ELISA | | | |

Specificity: 202/211 = 95.7%
Sensitivity: 250/254 = 98.4%

As can be seen from Tables 4-8, the Toxo IgG microtiter ELISA is both a sensitive and specific assay for the detection of Toxoplasma-specific IgG as demonstrated by the overall high relative diagnostic specificity (95.7%) and sensitivity (98.4%) (Table 8) of the assay. The Toxo recombinant antigen cocktail comprised of the Toxo antigens P29, P30 and P35, in combination with the Toxo IgG assay, is both necessary and sufficient to replace the tachyzoite for the detection of Toxoplasma-specific IgG antibody.

Furthermore, there are several advantages of the recombinant antigen cocktail over the tachyzoite antigen for use in detection of IgG antibodies. First, the antigens are purified, and the amount of each antigen loaded into the immunoassay can be accurately determined and standardized, e.g., protein concentration. This minimizes interlot differences commonly observed in kits manufactured with different tachyzoite antigen lots. Hence, different lots of kits manufactured with different antigen cocktail lots will be very consistent from lot to lot. Secondly, mouse monoclonal antibodies to the individual recombinant Toxo antigens are used to monitor coating of the proteins to the solid phase. This further ensures that each lot produced is consistent. Third, the true clinical sensitivity of the assay using the purified antigens will be higher by virtue of the fact of the higher specific activity of the purified antigens. Finally, kits manufactured with the antigen cocktail are more stable during storage over time, and the performance of the assay using these antigens remains consistent over the shelf life of the assay. Kits manufactured with the tachyzoite antigen are not as stable and their performance may vary over time.

Additionally, there are many advantages of using a cocktail over using a single antigen alone. For example, an immune response to infection varies by individual. Some individuals produce antibodies to P35 and not to P66, whereas some individuals produce antibodies to P66 and not to P35. Thus, the antigen cocktail of the present invention will detect both groups of individuals.

Moreover, immune responses vary with time. For example. One individual may produce antibodies against P35 first and then later produce antibodies to only P66. Thus, the present cocktail will detect both types of “positive” individuals.

Furthermore, individuals may be infected with different Toxo serotypes, strains or isolates. Thus, the immune response may be such that multiple antigens are needed to detect the presence of all antibodies being produced. Again, the present cocktail allows for such detection.

Also, it is known from previous Western Blot experiments with tachyzoite proteins that the immune response to Toxoplasma is directed against several antigens. Once again, the present antigen cocktail will allow for the detection of all antibodies produced in response to these antigens.

Step F: Results of the Evaluation of Human Sera in the Recombinant Toxo Antigen (P29+P35+P66) IgM Microtiter ELISA

The serum specimens from Groups 1-4 (Example 12C) were tested for the presence of Toxoplasma-specific IgM using the recombinant Toxo antigen IgM microtiter ELISA (rpToxo-P29 (P29)+rpToxo-P35S (P35)+rpToxo-P66g (P66)). The results from this evaluation are presented in Tables 9-13.

TABLE 9
Evaluation of Group 1 Negative Serum Specimens by
Toxo IgM Microtiter ELISA

Abbott Imx Toxo IgM

| | | Pos | Neg |
|---|---|---|---|
| Toxo IgM | Pos | 0 | 7 |
| (P29 + P35 + P66) | Neg | 0 | 193 |
| Microtiter | | | |
| ELISA | | | |

Specificity: 193/200 = 96.5%

TABLE 10
Evaluation of Group 2 “Ancienne” Serum Specimens by
Toxo IgM Microtiter ELISA

Abbott Imx Toxo IgM

| | | Pos | Neg |
|---|---|---|---|
| Toxo IgM | Pos | 0 | 8 |
| (P29 + P35 + P66) | Neg | 0 | 92 |
| Microtiter | | | |
| ELISA | | | |

Specificity: 92/100 = 92.0%

TABLE 11
Evaluation of Group 3 “Évolutive” Serum Specimens by
Toxo IgM Microtiter ELISA

IC IgM

| | | Pos | Neg |
|---|---|---|---|
| Toxo IgM | Pos | 69 | 0 |
| (P29 + P35 + P66) | | | |
| Microtiter | | | |
| ELISA | Neg | 30 | 0 |

Sensitivity: 69/99 = 70.0%

TABLE 12
Evaluation of Group 4 “Précoce” Serum Specimens by
Toxo IgM Microtiter ELISA

IC IgM

| | | Pos | Neg |
|---|---|---|---|
| Toxo IgM | Pos | 53 | 1 |
| (P29 + P35 + P66) | | | |
| Microtiter | | | |
| ELISA | Neg | 2 | 10 |

Sensitivity: 53/55 = 96.7%

TABLE 13
Summary of Evaluation of Groups 1-4 Serum Specimens
by Toxo IgM Microtiter ELISA

Reference Test

| | | Pos | Neg |
|---|---|---|---|
| Toxo IgM | Pos | 122 | 16 |
| (P29 + P35 + P66) | | | |
| Microtiter | | | |
| ELISA | Neg | 32 | 295 |

Specificity: 295/311 = 94.9%
Sensitivity: 122/154 = 79.2%

As can be seen from Tables 9-13, the Toxo IgM microtiter ELISA is a specific assay for the detection of Toxoplasma-specific IgM as demonstrated by the overall high relative diagnostic specificity (94.9.%) (Table 13) of the assay. However, the assay appeared to be relatively insensitive to detection of Toxoplasma-specific IgM present in serum specimens from Group 3 “evolutive” (relative diagnostic sensitivity=70%, Table 11) but sensitive to detection of Toxoplasma-specific IgM present in serum specimens from Group 4 “précoce” (relative diagnostic sensitivity=96.7%, Table 12). These data suggest that the Toxo IgM microtiter ELISA may be more sensitive to the detection of Toxoplasma-specific IgM indicative of an acute or recent infection than the IC-M immunocapture assay used as the reference assay.

Further resolution testing was performed with the Abbott Imx Toxo IgM assay and a Toxo IgG avidity assay on the 30 discordant specimens listed in Table 11 that were positive for IgM antibody using the IC-M immunocapture assay and negative for IgM antibody by the Toxo IgM microtiter ELISA. Of the 30 specimens that were false negative by the Toxo IgM microtiter assay, 11 were resolved true negative by the Abbott Imx Toxo IgM assay. Furthermore, all 11 specimens contained Toxoplasma IgG with elevated avidity, representative of a past infection. Of the remaining 19 specimens that were false negative by the Toxo IgM microtiter assay, an additional 11 specimens corresponded to Toxoplasma infections which probably occurred greater than 6 months ago, as demonstrated by the presence of Toxoplasma-specific IgG high avidity antibodies. In addition, one specimen was from a patient with reactivation of toxoplasmosis where normally Toxo IgM antibodies are absent (an IC-M and Abbott Imx Toxo IgM false positive), and one specimen was from a patient with congenital toxoplasmosis. Therefore, after resolution by the Abbott Imx Toxo IgM assay followed by consideration of the Toxo IgG avidity data and clinical history of the specimens, of the 32 specimens false negative by the microtiter IgM assay, 11 were resolved true negative, 13 specimens (from congenitally infected patients) were removed from the calculation of relative diagnostic specificity and sensitivity, and 6 specimens remained false negative. The resolved data and recalculated sensitivity and specificity for the Toxo IgM microtiter assay are shown in Tables 14 and 15.

TABLE 14
Evaluation of Group 3 “Évolutive” Serum Specimens by
Toxo IgM Microtiter ELISA
After Resolution of Discordant Specimens

IC IgM

| | | Pos | Neg |
|---|---|---|---|
| Toxo IgM | Pos | 69 | 0 |
| (P29 + P35 + P66) | | | |
| Microtiter | | | |
| ELISA | Neg | 6 | 11 |

Sensitivity: 69/75 = 92.0%

TABLE 15
Summary of Evaluation of Groups 1-4 Serum Specimens
by Toxo IgM Microtiter ELISA
After Resolution of Discordant Specimens

Reference Test

| | | Pos | Neg |
|---|---|---|---|
| Toxo IgM | Pos | 122 | 16 |
| (P29 + P35 + P66) | | | |
| Microtiter | | | |
| ELISA | Neg | 8 | 306 |

Specificity: 306/322 = 95.8%
Sensitivity: 122/130 = 93.8%

As can be seen from Tables 14 and 15 after resolution of discordant specimens, the Toxo IgM microtiter ELISA configured with the antigen cocktail is both a sensitive and specific assay for the detection of Toxoplasma-specific IgM as demonstrated by the overall high relative diagnostic specificity (95.0%) and sensitivity (93.8%) (Table 15) of the assay. The Toxo recombinant antigen cocktail comprised of the Toxo antigens P29, P35, and P66 is both necessary and sufficient to replace the tachyzoite for the detection of Toxoplasma-specific IgM indicative of a recent toxoplasmosis.

Furthermore, there are several advantages of this recombinant antigen cocktail over the tachyzoite antigen for use in detection of antibodies to IgM. First, the antigens are purified and the amount of each antigen loaded into the immunoassay can be accurately determined and standardized, e.g., protein concentration. This minimizes interlot differences commonly observed in kits manufactured with different tachyzoite antigen lots. Hence, different lots of kits manufactured with different antigen cocktail lots will be very consistent from lot to lot. Secondly, mouse monoclonal antibodies to the individual recombinant Toxo antigens are used to monitor coating of the proteins to the solid phase. This further ensures that each lot produced is consistent. Third, the true clinical sensitivity of the assay using the purified antigens will be higher by virtue of the fact of the higher specific activity of the purified antigens. Fourth, an IgM assay with the antigen cocktail will preferentially detect IgM antibodies produced in response to a recent infection. This can be seen in Tables 11 and 14 where specimens with high avidity IgG antibodies (indicative of a past or chronic infection) were negative for Toxo-specific IgM using the antigen cocktail in a microtiter ELISA. Finally, kits manufactured with the antigen cocktail are more stable during storage over time, and the performance of the assay using these antigens remains consistent over the shelf life of the assay. Kits manufactured with the tachyzoite antigen are not as stable, and their performance may vary over time.

Additionally, there are many advantages of using a cocktail over using a single antigen alone. For example, an immune response to infection varies by individual. Some individuals produce antibodies to P35 and not to P30 whereas some individuals produce antibodies to P30 and not to P35. Thus, the antigen cocktail of the present invention will detect both groups of individuals.

Also, immune responses vary with time. For example, one individual may produce antibodies against P35 first and then later produce antibodies to only P30. Thus, the present cocktail will detect both types of “positive” individuals.

Furthermore, individuals may be infected with different Toxo serotypes, strains or isolates. Thus, the immune response may be such that multiple antigens are needed to detect the presence of all antibodies being produced. Again, the present cocktail allows for such detection.

Also, it is knownn from previous Western Blot experiments with tachyzoite proteins that the immune response to Toxoplasma is directed against several antigens. Once again, the present antigen cocktail will allow for the detection of all antibodies produced in response to these antigens.

### EXAMPLE 13

### Immunoblot Analysis of T. gondii Lysate Antigens

T. gondii lysate antigens were prepared from tachyzoites of the RH strain. The parasites were harvested from the peritoneal cavity of Swiss-Webster mice, as previously described (Prince et al., Molecular Biochemical Parasitology 43:97-106 (1990)). Reduced lysate was prepared by resuspension of tachyzoites in reducing sample buffer containing 0.5% sodium dodecyl sulfate (SDS), 25 mM Tris-HCl, pH 6.8, 170 mm β-mercaptoethanol, 8.4% glycerol, and 0.01% bromophenol blue. Non-recombinant CKS and rPRoxo-P35S proteins were prepared in reducing sample buffer containing 0.5% sodium dodecyl sulfate (SDS), 25 mM Tris-HCl, pH 6.8, 170 mM 2-mercaptoethanol, 8.4% glycerol, and 0.01% bromophenol blue. All samples were boiled for 5 minutes. Proteins were separated by SDS-PAGE in 10% slab gels and transferred to nitrocellulose membrane. For immunoblot analyses with human sera, the membranes with reduced rPToxo-P35S antigen or non-recombinant CKS antigen were incubated with pools of sera that had been diluted 1:100 in PBS-0.5% Tween 20 (PBS-T) containing 5% nonfat dry milk (Sambrook et al., 1989, Molecular Cloning. A Laboratory Manual, 2^{nd} ed. Cold Spring Harbor Laboratory Press, Cold Spring Harbor, N.Y.). The conjugate used was HRPO-conjugated goat anti-human IGG (Caltag Laboratories) at a previously determined optimal dilution of 1:3000 in PBS-T containing 3% bovine serum albumin (BSA). The substrate, 3,3′-diaminobenzidine tetrahydrochloride (Sigma Chemical Company, St. Louis, Mo.), was used at a final concentration of 0.1 mg/ml in PBS. Control immunoblots performed to test for the reactivity of the conjugates to either rPToxo35-P35S antigen or non-recombinant CKS antigen did not reveal any bands.

The results demonstrate that IgG antibodies from sera from humans with a T. gondii infection are reacting to a protein of the correct size to be the P35 fusion protein and not an irrelevant E. coli protein.

### EXAMPLE 14

### Preparation of Serum Samples and Performance of ELISA

Serum Samples:

Sera were provided by the Toxoplasma Serology Laboratory of the Palo Alto Medical Foundation (Palo Alto, Calif.) and had been stored frozen for no longer than 2 years. The samples were from 141 pregnant women and were divided into three groups based on their selogic test results: Group I was composed of sera from 41 women with a serologic profile consistent with a recently acquired T. gondi infection (acute profile) and Group II of sera from 50 women with a serologic profile consistent with chronic infection. The serological tests used to classify these sera were: the Sabin Feldman dye test (DT), the double-sandwich-IgM ELISA (IgM ELISA), and the double-sandwich-IgA ELISA (IgA ELISA), and the AC/HS test (Lisenfeld et al., Journal of Clinical Microbioloqy 34:2526-30(1996); Lisenfeld et al., Journal of Clinical Microbiology 35:174-78 (1997); Wong et al., Clinical Infectious Diseases 18:853-62 (1994)). These tests comprise the “toxoplasma serological profile” (Lisenfeld et al., Journal of Clinical Microbiology 35:174-78 (1997)). Sera from women in Group I had high DT titers (from 1:256 to 1:32,000), positive IgM ELISA titers (from 2.3 to 9.7), positive IgA ELISA titers (from 1 to >28), and acute patterns in the AC/HS test. Sera from women in Group II had low DT (from 1:16 to 1:512), negative IgM ELISA titers (from 0 to 0.8), and chronic patterns in the AC/HS test. The classification of acute or chronic profile was based on the individual's clinical history as well as the combination of the results of the toxoplasma serological profile (Lisenfeld et al., Journal of Clinical Microbiology 35:174-78 (1997); Lisenfeld et al., Journal of Clinical Microbiology 35:174-78 (1997)). An additional group (Group III) was composed of sera from 50 women who were seronegative for T. gondii antibodies in the DT. A pool of serum samples from 5 seronegative individuals, each of whom was negative when their sera were tested undiluted in the DT, was used a negative control for immunoblots and the ELISA. Serum from a patient with a recently acquired toxoplasmic lymphadenopathy was used as a positive control on each ELISA plate.

ELISA:

Each well of a microtiter plate (Nunc, Roskilde, Denmark) was coated with 0.1 ml of a 10 μg/ml of rPToxo-P35S antigen was determined to be the optimal concentration with which to coat the wells of the ELISA plates. Consequently, the control non-recombinant CKS antigen preparation was also used at 10 μg/ml to coat plates. After incubation at 4° C. overnight, the plates were washed three times with PBS-T and post-coated with 200 μl per well of 3% BSA in PBS-T at 37° C. for 2 h. The plates were then washed and 100 μl of test or control serum diluted 1:50 in 1 BSA in PBS-T were applied to each well with rPToxo-P35S antigen preparation, non-recombinant CKS antigen preparation or without antigen. Plates were incubated at 37° C. for 1 h, washed and then 100 μl of HRPO-conjugated goat anti-human IgG at a dilution of 1:1000 was added to each well. The plates were incubated at 37° C. for 1 h, washed and then 100 μl of 0.03% O-phenylenediamine in H₂O₂ were added to each well. The optical density values were measured with an automatic ELISA reader (Dynatech Laboratories, Chantilly, Va.) after 15 min. incubation at room temperature. Each sample was run in duplicate wells. Results were determined for each patient by taking the mean value of the absorbency readings of duplicate wells.

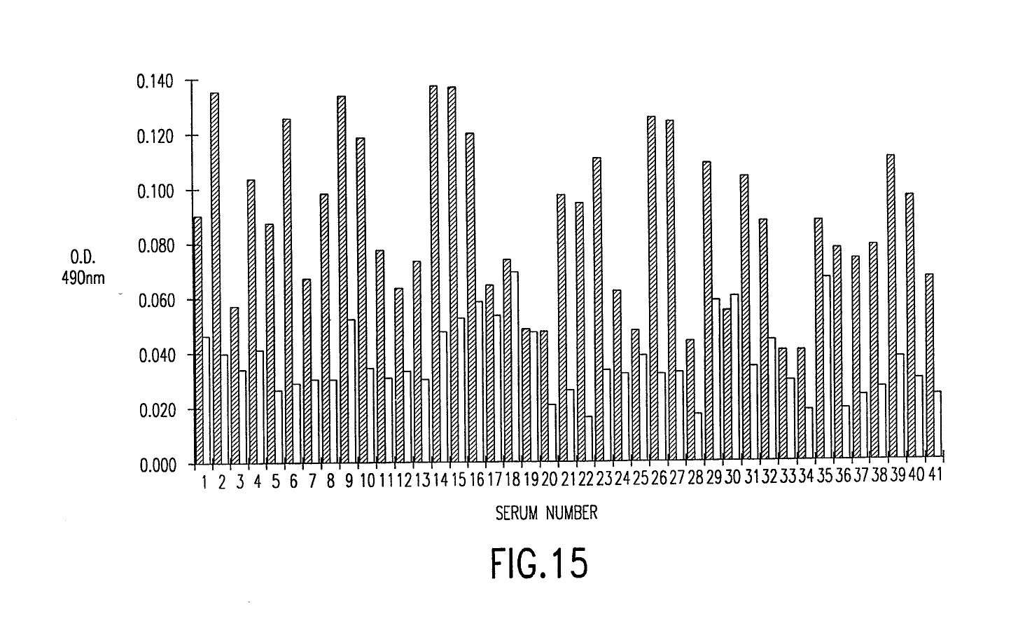

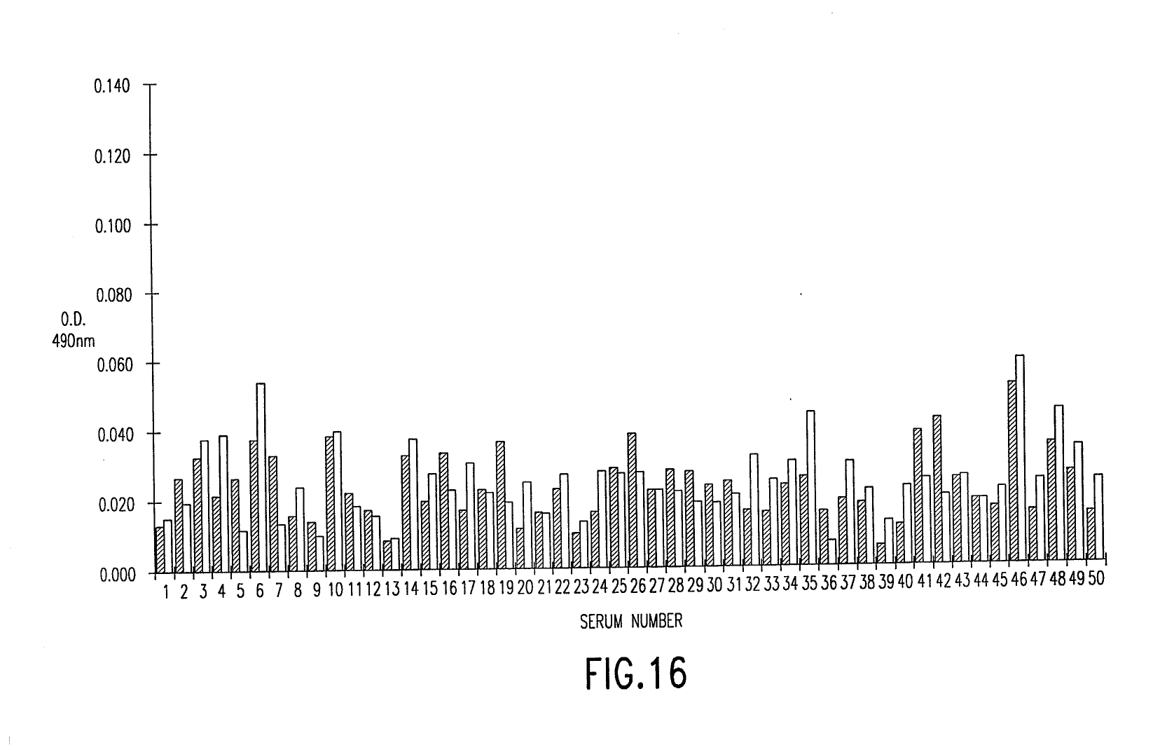

Of the 41 sera from Group I, 40 (97.6%) had absorbency readings higher in the rPToxo-P35S ELISA than in the control ELISA and 1 had absorbency readings higher in the control ELISA than in the rpToxo-P35S ELISA (FIG. 15). In contrast, of the 50 sera from Group II, 30 (60%) had readings in the control ELISA that were equal to or higher than in the rpToxo-P35S ELISA, and the remaining 20 (40%) had absorbency readings in the rpToxo-P35S ELISA that were only slightly higher than the readings noted in the control ELISA (FIG. 16). The mean of the Group I seara (0.0513+/−0.0045 standard error) was significantly (p=0.0001) higher than the mean of the Group II sera (0.0031+/−0.0008 standard error).

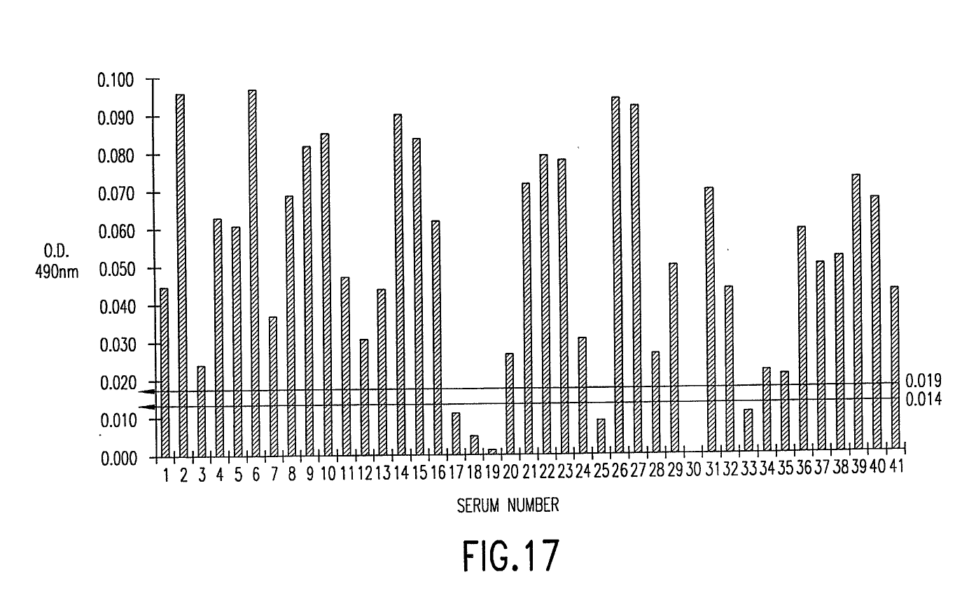

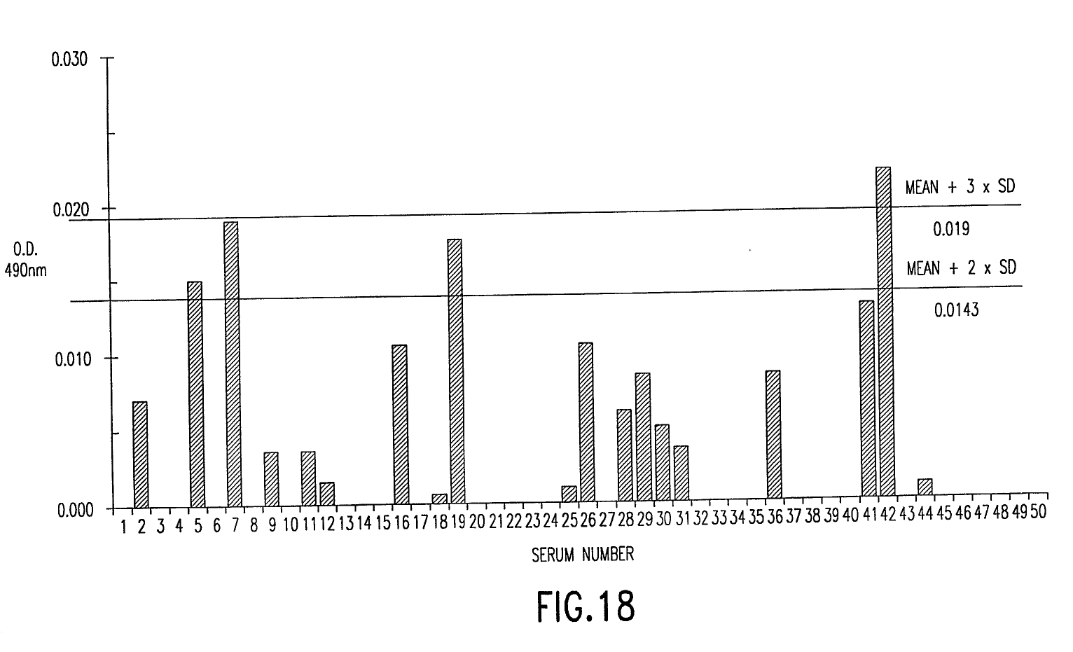

With respect to determining whether the reactivity of IgG antibodies with rpToxo-P35S could be used to differentiate Group I from Group II sera, it was observed that 35 (85.3%) of 41 Group I sera had normalized readings higher than the cut-off value (FIG. 17). In contrast, only 4 (8%) of the 50 Group II sera had normalized readings higher than the cut-off value (FIG. 18). When compared with interpretations made based on the toxoplasma serological profile results, the sensitivity of the rpToxo-P35S ELISA for recently acquired infection was 85.3% and the specificity was 92%. Using a cut-1off value (0.019) based on the mean plus 3 standard deviations of the Group II readings, 35 (85.3%) of 41 Group I sera (FIG. 17) and only 1 (2%) of the 50 Group II sera (FIG. 18) had normalized readings higher than the cut-off value.

The above results demonstrate that the P35 antigen in the IgG ELISA can be used to distinguish between patient sera obtained from individuals in the acute stage of infection versus individuals in the chronic stage of infection. In particular, it was determined that the patients of Group I has an acute infection and those of Group II had a chronic infection. Thus, P35 may be used to distinguish between acute and chronic Toxoplasmosis.

1. A composition comprising Toxoplasma gondii antigens P29, P30 and P35.

2. A composition comprising Toxoplasma gondii antigens P29, P35 and P66.

3. The composition of claim 1 or 2 wherein said composition is a diagnostic reagent.

4. The composition of claim 1 or 2 wherein said antigens are produced by recombinant or synthetic means.

5. An isolated nucleic acid sequence represented by SEQ ID NO: 26.

6. A purified polypeptide having the amino acid sequence represented by SEQ ID NO: 27.

7. A polyclonal or monoclonal antibody directed against said purified polypeptide of claim 6.

8. A method for detecting the presence of IgM antibodies to Toxoplasma gondii in a test sample comprising the steps of: a) contacting said test sample suspected of containing said IgM antibodies with a composition comprising P29, P35 and P66; and b) detecting the presence of said IgM antibodies.

9. A method for detecting the presence of IgM antibodies to Toxoplasma gondii in a test sample comprising the steps of: a) contacting said test sample suspected of containing said IgM antibodies with a composition comprising antigen P29, P35 and P66 for a time and under conditions sufficient for the formation of IgM antibody/antigen complexes; b) adding a conjugate to the resulting IgM antibody/antigen complexes for a time and under conditions sufficient to allow said conjugate to bind to the bound antibody, wherein said conjugate comprises an antibody attached to a signal generating compound capable of generating a detectable signal; and c) detecting the presence of IgM antibodies which may be present in said test sample by detecting a signal generated by said signal generating compound.

10. The method according to claim 9 wherein said composition further comprises P30.

11. A method for detecting the presence of IgG antibodies to Toxoplasma gondii in a test sample comprising the steps of: a) contacting said test sample suspected of containing said IgG antibodies with a composition comprising P29, P30 and P35; and b) detecting the presence of said IgG antibodies.

12. A method for detecting the presence of IgG antibodies to Toxoplasma gondii in a test sample comprising the steps of: a) contacting said test sample suspected of containing said IgG antibodies with a composition comprising antigen P29, P30 and P35 for a time and under conditions sufficient for formation of IgG antibody/antigen complexes; b) adding a conjugate to resulting IgG antibody/antigen complexes for a time and under conditions sufficient to allow said conjugate to bind to bound antibody, wherein said conjugate comprises an antibody attached to a signal generating compound capable of generating a detectable signal; and c) detecting IgG antibodies which may be present in said test sample by detecting a signal generated by said signal generating compound.

13. The method according to claim 12 wherein said composition further comprises P66.

14. A method for detecting the presence of IgM antibodies to Toxoplasma gondii in a test sample comprising the steps of: a) contacting said test sample suspected of containing said IgM antibodies with anti-antibody specific for said IgM antibodies for a time and under conditions sufficient to allow for formation of anti-antibody/IgM antibody complexes; b) adding a conjugate to resulting anti-antibody/IgM antibody complexes for a time and under conditions sufficient to allow said conjugate to bind to bound antibody, wherein said conjugate comprises a composition comprising P29, P35 and P66, each attached to a signal generating compound capable of generating a detectable signal; and c) detecting IgM antibodies which may be present in said test sample by detecting a signal generated by said signal generating compound.

15. The method according to claim 14 wherein said composition further comprises P30.

16. A method for detecting the presence of IgG antibodies to Toxoplasma gondii in a test sample comprising the steps of: a) contacting said test sample suspected of containing said IgG antibodies with anti-antibody specific for said IgG antibodies for a time and under conditions sufficient to allow for formation of anti-antibody/IgG antibody complexes; b) adding a conjugate to resulting anti-antibody/IgG antibody complexes for a time and under conditions sufficient to allow said conjugate to bind to bound antibody, wherein said conjugate comprises a composition comprising P29, P30 and P35, each attached to a signal generating compound capable of generating a detectable signal; and c) detecting IgG antibodies which may be present in said test sample by detecting a signal generated by said signal generating compound.

17. The method according to claim 16 wherein said composition further comprises P66.

18. A vaccine comprising: 1) Toxoplasma gondii antigens P29, P30 and P35 and 2) a pharmaceutically acceptable adjuvant.

19. A vaccine comprising: 1) Toxoplasma gondii antigens P29, P35 and P66 and 2) a pharmaceutically acceptable adjuvant.

20. A kit for determining the presence of IgM antibodies to Toxoplasma gondii in a test sample comprising:
a) a composition comprising Toxoplasma gondii antigens P29, P35 and P66; and
b) a conjugate comprising an antibody attached to a signal generating compound capable of generating a detectable signal.

21. A kit for determining the presence of IgG antibodies to Toxoplasma gondii in a test sample comprising:
a) a composition comprising Toxoplasma gondii antigens P29, P30 and P35; and
b) a conjugate comprising an antibody attached to a signal generating compound capable of generating a detectable signal.

22. A kit for determining the presence of IgM antibodies to Toxoplasma gondii in a test sample comprising:
a) an anti-antibody specific for IgM antibody; and
b) a composition comprising Toxoplasma gondii antigens P29, P35 and P66.

23. A kit for determining the presence of IgM antibodies to Toxoplasma gondii in a test sample comprising:
a) an anti-antibody specific for IgM antibody;
b) a conjugate comprising: 1) Toxoplasma gondii antigens P29, P35 and P66, each attached to 2) a signal generating compound capable of generating a detectable signal.

24. A kit for determining the presence of IgG antibodies to Toxoplasma gondii in a test sample comprising:
a) an anti-antibody specific for IgG antibody; and
b) a composition comprising Toxoplasma gondii antigens P29, P35 and P66.

25. A kit for determining the presence of IgG antibodies to Toxoplasma gondii in a test sample comprising:
a) an anti-antibody specific for IgG antibody;
b) a conjugate comprising: 1) Toxoplasma gondii antigens P29, P35 and P66, each attached to 2) a signal generating compound capable of generating a detectable signal.

26. A method for detecting the presence of IgM antibodies to Toxoplasma gondii in a test sample comprising the steps of: (a) contacting said test sample suspected of containing IgM antibodies with anti-antibody specific for said IgM antibodies for a time and under conditions sufficient to allow for formation of anti-antibody IgM complexes; (b) adding antigen to resulting anti-antibody/IgM complexes for a time and under conditions sufficient to allow said antigen to bind to bound IgM antibody, said antigen comprising a mixture of P29, P35 and P66; and (c) adding a conjugate to resulting anti-antibody/IgM/antigen complexes, said conjugate comprising a composition comprising monoclonal or polyclonal antibody attached to a signal generating compound capable of generating a detectable signal; and (d) detecting IgM antibodies which may be present in said test sample by detecting a signal generated by said signal generating compound.

27. The method according to claim 26 wherein said mixture further comprises P30.

28. A method for detecting the presence of IgG antibodies to Toxoplasma gondii in a test sample comprising the steps of: (a) contacting said test sample suspected of containing IgG antibodies with anti-antibody specific for said IgG antibodies for a time and under conditions sufficient to allow for formation of anti-antibody IgG complexes; (b) adding antigen to resulting anti-antibody/IgG complexes for a time and under conditions sufficient to allow said antigen to bind to bound IgG antibody, said antigen comprising a mixture of P29, P30 and P35; and (c) adding a conjugate to resulting anti-antibody/IgG/antigen complexes, said conjugate comprising a composition comprising monoclonal or polyclonal antibody attached to a signal generating compound capable of generating a detectable signal; and (d) detecting IgG antibodies which may be present in said test sample by detecting a signal generated by said signal generating compound.

29. The method according to claim 28 wherein said mixture further comprises P66.

30. A method for detecting the presence of IgM and IgG antibodies to Toxoplasma gondii in a test sample comprising the steps of: a) contacting said test sample suspected of containing said IgM and IgM antibodies with a composition comprising antigen P29, P30, P35 and P66 for a time and under conditions sufficient for the formation of IgM antibody/antigen complexes; b) adding a conjugate to the resulting IgM antibody/antigen complexes and IgG antibody/antigen complexes for a time and under conditions sufficient to allow said conjugate to bind to the bound IgM and IgG antibody, wherein said conjugate comprises an antibody attached to a signal generating compound capable of generating a detectable signal; and c) detecting the presence of IgM and IgM antibodies which may be present in said test sample by detecting a signal generated by said signal generating compound.

31. A method for detecting the presence of IgM and IgG antibodies to Toxoplasma gondii in a test sample comprising the steps of: a) contacting said test sample suspected of containing said IgM and IgG antibodies with anti-antibody specific for said IgM antibodies and said IgG antibodies for a time and under conditions sufficient to allow for formation of anti-antibody/IgM antibody complexes and anti-antibody/IgG antibody complexes; b) adding a conjugate to resulting anti-antibody/IgM antibody complexes and resulting anti-antibody/IgG antibody complexes for a time and under conditions sufficient to allow said conjugate to bind to bound antibody, wherein said conjugate comprises P29, P30, P35 and P66, each attached to a signal generating compound capable of generating a detectable signal; and c) detecting IgM and IgG antibodies which may be present in said test sample by detecting a signal generated by said signal generating compound.

32. A method for detecting the presence of IgM and IgG antibodies to Toxoplasma gondii in a test sample comprising the steps of: (a) contacting said test sample suspected of containing IgM and IgG antibodies with anti-antibody specific for said IgM antibodies and with anti-antibody specific for said IgM antibodies for a time and under conditions sufficient to allow for formation of anti-antibody/IgM complexes and anti-antibody/IgG complexes; (b) adding antigen to resulting anti-antibody/IgM complexes amd resulting anti-antibody/IgG complexes for a time and under conditions sufficient to allow said antigen to bind to bound IgM antibody, said antigen comprising a mixture of P29, P30, P35 and P66; and (c) adding a conjugate to resulting anti-antibody/IgM/antigen complexes and anti-antibody/IgG/antigen complexes, said conjugate comprising a composition comprising monoclonal or polyclonal antibody attached to a signal generating compound capable of generating a detectable signal; and (d) detecting IgM and IgG antibodies which may be present in said test sample by detecting a signal generated by said signal generating compound.

33. A method of producing monoclonal antibodies comprising the steps of:
a) injecting a non-human mammal with an antigen;
b) administering a composition comprising antibiotics to said non-human mammal;
c) injecting said non-human mammal with said antigen;
d) fusing spleen cells of said non-human mammal with myeloma cells in order to generate hybridomas; and
e) culturing said hybridomas under sufficient time and conditions such that said hybridomas produce monoclonal antibodies.

34. The method of claim 34 wherein said antigen is derived from an organism selected from the group consisting of Borrelia burgdorferi, Schistosoma treponema, Toxoplasma gondii, Plasmodium vivax and Plasmodium falciparum.

35. A composition comprising the isolated nucleic acid sequence represented by FIG. 11 or a fragment thereof.

36. A composition comprising amino acids 1-135 of P35.

37. The composition of claim 35 or 36 wherein said composition is a diagnostic reagent.

38. A method for distinguishing between acute and chronic Toxoplasmosis in a patient suspected of having either said acute or chronic Toxoplasmosis comprising the steps of: a) contacting a test sample, from said patient, with a composition comprising amino acids 1-135 of P35; and b) detecting the presence of IgG antibodies, presence of said IgG antibodies indicating acute Toxoplasmosis in said patient and lack of said IgG antibodies indicating chronic Toxoplasmosis in said patient.

39. A kit for distinguishing between acute and chronic Toxoplasmosis in a patient suspected of having either said acute Toxoplasmosis or said chronic Toxoplasmosis comprising:
a) a composition comprising amino acids 1-135 of Toxoplasma gondii antigen P35; and
b) a conjugate comprising an antibody attached to a signal generating compound capable of generating a detectable signal.

40. A kit for distinguishing between acute and chronic Toxoplasmosis in a patient suspected of having either said acute Toxoplasmosis or said chronic Toxoplasmosis comprising:
a) an anti-antibody specific for IgG antibody; and
b) a conjugate comprising amino acids 1-135 of Toxoplasma gondii antigen P35 attached to a signal generating compound capable of generating a detectable signal.
